SEQUENCE LISTING (101 sequences)

SEQ ID NO: 1 - DNA, 17021 bp, Homo sapiens
agcactttgggaagccaaggcaggtggattgcttgagctcaggagtttgaggccaccctgggcaacgtggcaaaatccagtctctacaaaaaacacaaaaattagctgggcacagtggtgcgcacctgctgtcccagctactcgggaggctgaggtgggaggatcacctgagcctgggaggtcaaggttgcagcaaaccaagatcctgccactgcactccagcctgggcaacagagcaaaaccctgtcaaaaaaaaaaaaaaaatctggtctaagcaccacctttgatggaggcaggaatcctgcagcagcctggagccaacccaatggctccctgcctctggtgctctatttcttatcaccatacaggtctctaaggtcacctttaggtctaacattgtatgaatgattgtcagcaactttccaatttttttcatatacatatattttttttattttattttttactttttctttttttgagacaggatctcactctgtcacccaggctggagtgcagtggcgtgatcatagctcactgtagcttcaacctcctgggcacaagtgatcctcccacctcagcttccaaggtacctgtgactacaggaatgcgccaccatgcccggctaattttgttatatatatatatatatatatttgtaccttggttttctcatctgtaacaccaggggttgaagtgctgtgatattttttggttctaggataatttggaaatagggatctcttaacatcttggatactgccaatagatcattcacgaaatccctttagatgagtgttatgtttacttaggtctctaaaacaatttgtaaggtcaacagatggggataagaatgacctgaagctggtcgtctttgttcgcaaccagaaacagtcctgcctcaaaaaaaaaaaaaaaaaaaagggagtaggcaggtggccatatttgtttctggcaagtgagtgctgaaggaaaggagctgaagcctcccacagtcataactggtgctggcaggctactgtctcggtcttgggcgccactgatctaaggtcacggctctgcttgctgctcccacccgctccagtttaaaacctgcggttccagggttctccagcccctccctttttcacgctccgaagccgagaaggcccaaagcgaagacagagaggacccggaagtagggaaaacctctgagcacgtgatgggggaacacgcgggtgctgtcacgtgatccgacaaacggcctctgcatagtgcagaacattctgctgctcttaaaggtacaggcctcagggtccctgctgtagacggggcgggggagagtacgatgggtggggcgtggtgggtcgtagggcgctcgagatggagcccccagcttccttgatggatcgcggggcgcgagtgccctagacaagccggagctgggaccggcaatcgggcgttgatccttgtcacctgtcgcagaccctcatccctcccgtgggagccccctttggacactctatgaccctggaccctcgggggacctgaacttgatgcgatgggaggctgtgcaggctcgcggcggcgcttttcggattccgagggtgagtattcccgcccaccctcatggaacgaccacctctggctcgcagcccacctgaggggaatgagagctgactcggccccgggaggacacgcaaggagcggtcgtgcacctgagagtaggtgggggtcatgccctcttctcgctctcccaggggaggagaccgtcccggagccccggctccctctgttggaccatcagggcgcgcattggaagaacgcggtgggcttctggtgagtggcctgagacttcagcgagtgacaatggcatgagaagagggaagtgaccggaggaaagggggaagaaagagttaagctgcgcaaaggagctgaacccacaaatatttactgaatccactggtttgccccaggggcagcatgtaaaatttccaaacttcactccttatcaccttccggatttagcaagatcctcatacctaccacctgtaggattatttacttttttcttccagccaattcagccttaacgttttttagtgcacatatttttaaaaagaaaactttaaatcacgactccacttggaaaaccagcgttttcctagtgaacgtttaaataaatgcatcactactaaaacttttttatttttttaatttattttattttatcttatttttttttctttttgagacagggtcttgctctgtttcccaggttggaatgcagtagtgcaatcacagctccctgcagcctggaactcctgggctcaagtgattctcccacctcagcctccaagtaggtgtggctacaggtgtgcaccaccgcatctgcctaatttttatatttttttatagagatgggggtctcactatgggacctgggctggtctcaaactcgtggcctcaagtgatcctcctacctcaaccttccaaagtgctgagattacaagcatgagccaccatgccgggcctgaacctttttttttttttttttttttgagacggagtctctctctgtcctccaggctggagtgcagtggcgcaatctcggctcactgcaaactccgcctcccaggttcacaccattcttttgcctcagcctccggagtagctgagactacaggcgcctgccaccacgcccggctatttttttgtatttttagtagagatggggtttcaccgcgttatccaggatggtctcgatctcctgacctcgtgatccgcccgcctcggcctcccaaagtgctgggattacaggtgtgagccaccgcgcccagctttttttttttttttttttaattttattttatttttgagacagagtctcactctatcacccaggctggagagcagtggcacaatctcggctcactgaaacctccacctccaaggttcaagctattctcctgtctcagctccccgagtagctgggattacaggtgcgtgccaccagacacagctaattttttgtatttttagtaaagacaggatttcaccatgttggtcaggctggtctagaactcctgaccttaggtgatccgcttgcctccacttcccaaagtgctgggattacaggcgtgagccaccgtgccccgccctgacataggggctttgggatcacagacttggattcacttccagcctccaaggcctctcccagaaactctctgtgaccactctccttttttttttttttttttgagatagagtctcgctcttgtagcccaggctggagtgcaatggcacaatctcggcttactgcaacctccgcctttcgggttcaagtggttctcctgcctcagcctcctgagtagctaggattacaggcatgtgcaacttcgcccagctaattttgtattttttagtagagacggggtttctccatattgctcaggctggtctcgaactcctgacctcaggcgatccgcccgcctcggcccctcaaagtgctggaattacaggtgtgagccactgcactcggcctcttcctttttattttttatttttgtgacagggtcttgctctgtcacccaggctagagtgcagtggcatgatcacagttcactgcagcctctgactcctggactcaagcaatcctcccacctcagcctcccaagtagctgggactacagtgcaagccaccacacctggctaatttttaaattttttgtagagatgggggtctcactatcttgcccaagcaccaaagcctgtatttttgaccccaacattgaatgaggatgggatctggtgatagagggaaaaaaaagagggggctgattgaagggcacaggtaagggaaggtttggcacaaaggctacagatggctgcaggatgaggagggagttgagacctgtcctgctccttccaggctgctgggcctttgcaacaacttctcttatgtggtgatgctgagtgccgcccacgacatccttagccacaagaggacatcgggaaaccagagccatgtaagtgactctctaccaccaccaccatggttagtccctgtgggaagatgagggggtgggacaaggtggggtaaagtttccagtctctggctgggcacagtggctcacacctgtaatcccagcagtttgggaggctgaggcgggcggatcacttgaagtcaggagttcgagaccagcctggccaacatggtgaaactccgactctactaaaaatacaaaaactacctgggtgtggtggcacacacctgtagtctgagctattcaggaggctgaggcaggagaattgcttgaagccaggaggtggaagttgcaatgagccaagatcacaccactgcactctagcttgggcaacagagtgagacaccatctcaaaaaaaagaaagctcttgaatgtgttatctaaacaaagtccatttacagggccatctgtggcctgtggtctagtagtgtgagactcatatggaaaccccctcttcattgtgcatgcagagacgctgaggtcctgagaagtcagtcactgctgacccaaagccatccttcaagtgaaggcagagctgggacgggagccaggctctgtgtgtctatccctctgcctccagggtgaaagacatgcttttctcccattaggtggacccaggcccaacgccgatcccccacaacagctcatcacgatttgactgcaactctgtctctacggctgtgcgtactcatctcacctggtccttgcctgacccagggcccctgggtctatagaccccactcccagcccttcactacccagctgggactgtcatgattaaaacagttgagacctgggctgggcgcactggctcacacttgtaatcccagcactttgggagaccaaggtgggaggatcacttgagcctaggagtttgagaccagcctgggcagtatagtgagacccccatcttaaagaaaaaaattaaaaaatatatatatataaaataattgagaccttaatataatttctgaggctgaggcggatggatcacttgagaccaggagttcaagaccagcctggccaacatggtgaaaccccatctgtactaaaaatacaaaaattagccaggcatggtggtgcacgcctgtaatcccagctactcaggaggctgaggcaggagaatcacttgaacccaggaggtggaggttgtagtgagccaagatcgagccactacactccagcctgggtggcagaatgagactcactctcaaaatatatatatatatagtttctgagtcctttctgtctgcaccatatattagctcatttaagccacacagcagtcctgtgagctaggtgctatgatattcccattttccagatgaggaaactgaagctcagagagtataaactcttgacacacaactaaggagtgggagagctgagacttgaacccaggcgtgcctgactccagagcctgtgtttgtagcaggcctgtttggccagctcctgcctctccttggccacgtggttgggagggttgtcccctggaagctctgcggtctcactctattctcctgtcccaggctgtgctcctggcggacatcctccccacactcgtcatcaaattgttggctcctcttggccttcacctgctgccctacaggtctgggtgagggtagtgggaggcagggtgggcaggagctgagaaaggggaggctgggatggctgagatgctgagagtagagaccgaccttccccctcccttcccttctcaccccctcagcccccgggttctcgtcagtgggatttgtgctgctggaagcttcgtcctggttgccttttctcattctgtggggaccagcctgtgtggtgagtgtgtggttctgtgtcagatggggagccccgaggaaccacatcagagcatttgtgggaagagtctccccagcctcccagaggaaagggattcattctgtcacccttagaagcctgctagggctatcagcagtaggcgatgggagactgggacaatttggaggggtaggcagtggaggagatgggagaaaatggatgaattagatggagattgaggtgaacaaagtcaagactctgtgatggaccaggcacagtgactcatgcctataatcccagcactttggaaagccaaggcaggcagatcacctgaggtcaggagttcgaaaccagcctggccaacatggagaaaccccgtctctaccaaaaatacaaaaattagctgggtgtggtggcaggagcctgtaatcccagctactcgggaggctgaggcaggagaatctcttgaacctgggaggcagaggttgcagtgagccgagatcacgccactgtactccagcctgggtgacagggcgagactccgtctccaaaaagaaaagaaaagaaaaagactgatgaaggggcagagacatcaagggtgcatgtctgcccctggtctgataactgggtggatggaggtgccactctccatgaagggacacgcaggggagtggggctctgcttcagacctggaacctggcctatgcatgggatctattggagcctctatgagctgatactgaggaggccatggccagacacattagaggcctgggcagtgtggcaaggtgtggtgtgaccatcccagtgcttgtcctccccccaggtgtggtcttcgctagcatctcatcaggccttggggaggtcaccttcctctccctcactgccttctaccccaggtaagcaggtggagcagggagtgtggggagaggctgtcccatggtcagcctaggtcctcctgaatgttcctgtgttctccttcccagggccgtgatctcctggtggtcctcagggactgggggagctgggctgctgggggccctgtcctacctgggcctcacccaggccggcctctcccctcagcagaccctgctgtccatgctgggtatccctgccctgctgctggccaggtgagctgccctgagccgggagggagaggggtccaaggagagaaaacttggccatggctgggtgtggtggctcacgcctgtaatcccagcactttgggaggccaaggagggcagatcgcctaaggtcaggaaaccagcctggccaacatggtgaaaccccgtctctactaaaaatacaacaattagccaggtgtggtggcgggtgcctgtagtcccaactactcaggaggctaaggcaggagaatcgcttgaaccccggaggcagtggtggcagtgagccgagatcgtgccattgcactccagcccgggcgacagagttagactctgtctcaggaagaaaaaaaaaaaaagaaagaaaagaaaacttgattatgattgcaatcttcaagtccctaccttgctgtgaagggaggcggaatctggactctgatagccccaggtgtgagtcctggagctgccactttttagctttgtagcgttgaacaagttactccacctctctgaaccctcagtttccccatatctcaaatggcagttgttcttgctttccttggaggtgatgagggtaatgcattcagcacagtgtggttcccaaggtgattagaagtaggatgagggtggactttatttgattagttccttttttttttttttttttttttaatcagtgttgaccaggttggcctcgaatgtgtagccttgcctgcctgagtgccagggcaacaggcctgaaccatggcgactcccttttttttttttttttttttttttttttgagacggagtctcatggtgtcactcaggctggagtgcagtgactggtgtgatctcagctcactgcagcctcggcctcccgggctctagtgattctcctgcctcagcctcccgagtagctgggactacaggcccatgccaccatgcctggctaattttttatatttttagtagagacggggtttcactgtattggccaggctggtctcaaactcctgacctcaagtgatccacctgccttggcctcccataatgctaggaatacaggcgtgagccaccgcgcccggcctgattagttctgtgtattttcatgcatattacaaaacactttggccgggcatggtggctcacatctgtaatcccagcactttgggaggccgaggcgggcgcaccacgaggtcaggagtttgagaccagcctggccaatatggtgaaaccccgtctctactaaaaatacaaaaattagccaggcttggtggcgcttgcctgtagtcccagctacttgggaggctgaggagaagaatcgcttgaacccaggaggcagaggttgcagtgagccgtgattgtgccactgcactccaggctgggcaacagagcgagactccgtctaaaaaaaaaaaaaacacacactttgaactagccagacacacctggcccttcacaagattagtgcttaggacaagtctaagtagaaaaagtgagtccatttaaagaaaaatattaagtaaaaatatatatatacaggaggaatatggatatggcaaataacatgaagccagaacctaagtgactaaaatcgaggaagttctggcctagcacagagtgaacgtataattaatgggagctagagcgaccattagaataataatggccagaaaacagaactgactcgctaggtatgagccagcagtccgaacaagggcctgctgagcacagtggctcacagctgtaatcccagtgctttaggaggctgaggctggaggaccactagaggctagaaatttgagaccagcctgggcaacatagcgagactccatttatacaaaaaatggaaaatatgaggcgggcttggtggcacgtgcctgtagtccccgctacttgggaggctgaggcgggaggattgcttgagcctgggaggtcgcggctgcagtgagctatgattgcaccactgcattccagcctggatgctggagaaagaccctgtctctgaaaaaaatcaaaacaaaatgaaggcccatgaataggaataaggagataaattcaggtccaaagaaagaagcctttgtccacaattggagctctcccgcacagcgtaggagccttagaggcagtgagctacccatctttgaaagtgttcaaaggtagaggtgttccctgggaaaagtggcctagatggtccctggggaccttcccagccagtagggttttctgaccctgccttcatcctactcctagctatttcttgttgctcacatctcctgaggcccaggaccctggaggggaagaagaagcagagagcgcagcccggcagcccctcataagaaccgaggccccggagtcgaagccaggtaggagacacagaccctcagagaggtcactttctttctctctgggtttggccttttcctctctgcaataggcaaagttaagaggggaaagagagtgagtgtactggttatgggaaaagcctttgtctatttgagaaatacttgttgaggacgagcacagtggctcacgcctgtaatatcccagcactttgggaggctgaggcaggaggaccacttgagctcagaagtctgagtccagcctgggcaacagagcaagaccttgtcgcaaaaaaaaaaaaaaaaaaaaaaaaaagaaaagaaaggaaggaagggagggagagaagaagggatattaattgagtacttaccatgtgccaggcattccaatcacaagtgctgaggccctgcggtggggataagctgggatgttctagaaccagagaatggccagtgagactgggcagggtgggccaatggccatctgtggagctgtacaagagacattggccgggcgtggtggttcatacctgtaatcccagcactttgggaggctgaggcagatggatcatttgagatcaagagtttgagaccagtctggccaacatggtaaaaccccgtctctactaaaaataaaaaaattagccaggtgtggtggtgcatgcctgtagtcccagctacttgggaagctgaggcacgagaatcacttgaacccaggaggcagaggctgcagtgagctgagattgcaccactgcactgcagcctgggcaactgagtgagactctgtctcaaaaaataaaaaaaattaaaaatcaaagagacgtgaagaggtctcacctggttagcttttattttcatcatgataaagtatgtatattacttgaagtttaccattttattattttttatcttactttatttttttgaggtggagtttcgctcttgttgcccaggctggagtgcaatggcgcaatctcagctcactacaacctctgcctcctgggttcaagtgattctcctgcctcagccacccgagtagctgggactacagacacctgccaccacacatggctaatttttgtatttttagtagagatggggtttctccatgttggccagggtggtcttgaactcctgacctcaggtgatccgcccacctcagtctcccaaggtgctgggattacaagcatgagccactgcgcccagccattttaaccatttttaagtgtccaatccagtggcatggaagtgagttcccactgttgcgtggtctggttaattctgtcataccggtgcctctttgccgagtcttcagtgtgaaaacttcatctgcccttctctctctgttcccctgcaggctccagctccagcctctcccttcgggaaaggtggacagtgttcaaggttcggatgatggctggggtatgtccctgtggggcttgctcacctccaggccccccgcttatatcttctgccttttccagggtctgctgtggtacattgttcccttggtcgtagtttactttgccgagtatttcattaaccagggacttgtaagtgaggggtgctaggaggggtgtggaggtggcgattggggctgggacccacacagccccgtccatctcccctgtctggtatttgttgcagtttgaactcctctttttctggaacacttccctgagtcacgctcagcaataccgctggtaagaggagcgagggcagtgggctgggagggcgccgtggtgatgcagctgccctgcccagtaggcaccggggggagcgggatggtccctggaggagcctcctctcctccccccaaccctaacctcaggtaccagatgctgtaccaggctggcgtctttgcctcccgctcttctctccgctgctgtcgcatccgtttcacctgggccctggccctgctgcaggtaccaaacccctgcccctcacttcactcccaccttggctcccagcttggctcccagcttccccaaaccccctgcttcccactatcagtgggaagtgtaaatttttttttttttttttgagacagagtctcgctctgtcgtccaggctggagtgcagtagcgcaatcttggcttactgcaacctctgcctcccgggttcaagtgactctcctgcctcagtctcctgagtagctgggattacaggcgcccgccaccatacctggctaatttttgtatttttagtagagatgggattttgccatattggctggtcttgaactcttgacctcaggtgatctgccggcctcagcctcccaaagtgctgggattacagggaagtgtaaattgtacactatcatttcttagcacagggaaccaaagtccagcagagctccatgtaaaatgtatagcctcaggagccaagcaaaactgagcttcagtactcagccactgtgtgacttagggcaggtctcagaacctctctgagcctcaatttcctcatgtataaattgggtgtggccaatacctatttttcagggtagttgtaagaaatagagatgagagaaagaatcgaagaacaagattttgtagtgtgtgtgtgtgtgtgtgtgcgtgtgtgtgtgtgtgtgtgtgtgtgtgtgtgtgtgttttaagcattgggattgggccgggccaggtggctcatgcctgtaatcccagcactatgggaggctgaggcaggtggatcatttgagcttggtagttcaagtccagcctgggcaacatggcacaatcacatctctacaaaaattagccaggtgcagtggcgcacacctgtagtcttagctactgggaagctgaggtgggaggatcacttgaacccatgaggtcaaggctgcagtaaccaaggtcatgccactgcactccagcctaggcaatagagcgagaccctgtctcccagaaaaagaaaaaagaaaggaaaagtcggaaccttcctctgtcgcctaggctggagtacagtggcatgatcataggtcatttacctcaggcaaagagacaggctttcaaataatgtttctaactataagaaaatgtacagttcttttattctagatagttaattataatagaattggtgactttgaagtcagtgggttatttatcttgagggtggtgagcaaattcaactgcccttggcaaacagtagtttacagctttaagggcgcaaacacccttccttacaacagtctaataaaaggagtttcatataaccagcattcattgtttcaaggttacaaatccacagcccctgcaggtaactgttacaataaactgtcaactgtgacaggaaagtcttctgcctttgaaatttaggaacttggccagtcctggtggctcacacctgtaatcccagcactttgggaggccaaggcaggtgaatcacctgaggtcaggatcaggagtttgagaccagtctggccaacacagtgaaaccccgtctctattaaaaatgcaaaaattagttgggcgtggtggcacctgcctgtcatctcagctactcgctaggctgaggcaggagaattgcttgaactccggatatggaggttgcagtgagccgagattgcagtactgtactccagcctgggccacagagcgagactccatctccaaaaaaaaagacttaggaactcaattctgtgatagcaaaatacagaaatagagattatactttaggctaggtgttgtggctcacacctgtaatcccaatgcttttggaggctagggtgggaggatcactcgaagccaggagcttgagaccagactggacaacatagtgagaccccccacctctacaaaaaattaaaaaaaaaaataagccaggtgggagaattgcttgagcccagaagtttgagagagcagcctgggtaacatcacgagacctcatctctacaaaaaacaacaacagggtgtggtgacattcacctgtaatcccagctactcgggaggctgaggcaggaggatcgctttagcccaggagttccaggctgcagtgagccataatcatgccactgtaccccagcctgggtgagaccctatctcaaaaaaaaagattacactttcatattatggcaaaagcatgtaaatatcctccagcctccttccttatttatctgagcagtgctgcaagcccagggtttgtacccacaaaaccccaaatttataactcagggtgcttgtagataccactgaaacatgtagcttatggttttaagctacatgtttttagctataatgttttttgttttgttttgttttttgagatggagtctcgctctgttgcccaggctggaatgtagcggtgcaatcttggctcactgcaacccctgcctcccagattcaagcgattctcaagcctcagccttccgagtagctgggattacaggtgcgccaccacgcccagcaaattttttgttttgttttgttttgttttgttttttagagacagagttttgctcttgtagctcaggctagagtacaatggcgtgatcttggctcactgcaacctccgcctcccaggttcaggcgattcttctgcctcagcctcctgcgtagctgggattacaggcacgtgccaccacacctggctaatttttgtattttttagtagagatggggtttcgccatgttggccaggctggtcttgaactcctgacctcaggtgatccacccgccttggccttccaaagtgctgggattacaggtgtgagccaccacacctggctagaggtgtgtcctttttaatgctcatctttaagtctacctcagatttaatgaattaggacctctagggtaagccccgcttggggcatttgtacctagatccccaggcaattcttacatatacgaaaagtatgaaccgccacgaggaggcctacacactgcacaagaggcaatgtggcgtggtcagtgctgggaggatggcacctccttgagagactggggcacagaggtgggacctacaccctggggtggggatcgaagaggcttcctggccaggtgcggtggctcctgcctataatcccagcactttgggaggctgaggcaggggggcagcgactgcttgagaccaggagttcgagcccagctgaataggatgcccggctactcccaccttgttttaccatagtgaaccaacggtttttccaataggggcaattttgctgcccagcggatgtttggcaacgtctgcaggcatgctttggttgtcacaactgagggatgctatgggcatctagcgggcagaggccagggatcctgctaaatgcgctgccatatacaggacagccctcaccatgcagaacaaccagcaccaaacgccaagagtgctgcaattgagaaatcctggtttcgatggtcccacttcctttaagaagcctccatttttttttttttttttttttttttttttatgagacaggatcttgctctgtcacccaggctggagtgcagtggcacaatcatagcttactgcagcctccacctcctaggttcaagcaatcctcctgtctcagactcccgagtagctgggactgcaggtgcgtgccaccatgctcaactaatttttaaattttttgtagagacggagttttgttatgttgcccaggctggtctcgaactcctggcctcaagtgatcatcccaccttggtctcccaatgtgctggtattacctgcatgagccactgcaccaggctgaggttgagaattttttttttttttttttgggatggagtctctctctgttgcccaggctggagtgcagtgctgcgatcttggctcactgcagcctctgcctcccaggttcaagtgattctcctgcctcagccttccaagtagctgggactacagaaccactacacccagataatttttgtatttttagttgagatggggttttaccatgtttagtagagaccaggccaggctggtctcgaaatcctgacctcaggtgatctgcccacctcagcctcccaaagtgctgggattacaggtgtgagccaccacaccgggctggtgttgagattttatctagacctggcagccctctccttcaccagtaactcctaaaaccagggacccctggaggggaggccacctctccttccctgccccgccctggtcccaggcttagcctctccccctgttcacagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggaggtcagcattggccgggcaagggctgggggtggcctgtccagggacacccagggcagggatgtctgggactgaagcctcacccctgctctctgccctcccagaccagtgatgagcaccgggagtttgcaatggcggccacctgcatctctgacacactggggatctccctgtcggggctcctggctttgcctctgcatgacttcctctgccagctctcctgatactcgggatcctcaggacgcaggtcacattcacctgtgggcagagggacaggtcagacacccaggcccaccccagagaccctccatgaactgtgctcccagccttcccggcaggtctgggagtagggaagggctgaagccttgtttcccttgcaggggggccagccattgtctcccacttggggagtttcttcctggcatcatgccttctgaataaatgccgattttatccatggacttcttatatcgtttttgtctctaaaaagaaacttttattatgaaagtaatacatgcccactttcctatatatagacagcataaagaaggtatgtcagcagatttctcccttttttgtttgtttgtttgtttgtttgtttgttttgacagagtctcactctgtcacctaggctggagtgcaatggcgtgatcttggctcactgcaacctccacttcccggttcaagcaattctcctgcctcagcctcccgagtagctgggattacaggtggtcactaccatgtctggctaatttttatatttttagtacagacgacattttgccatgttggccaggctggtctcaaactcatgacctcaggtgatccgcccaccttggcctcccaaagtgctgagattacaggtgtgagccactgtacctggcctctgccatctttgaggccttacgtattcattcattcatgcattcattgtttgggacaatctgtaaacaatcatgttggacattctttccagcatgaaagttttcagagaagtggaaagaattgtgcagtgaatcatcctgagtatgcgccaccctgttctatgttaacattttgttacagttgttttatcatgtagtccagcccccatcaaatctatgtattcactgttccatcagtgagtagatagagctaatgaatggcagggttggcacctgaggcattttattttattatttttttttatttttagagacagggttgcactctgtcacccaggctgaagtacagtggcacagtcatggctcactgtagccttgacctcctagactcaaacaatcctcacaccttggcctcccaagcagctgggactgcaagtgcatgccaccaagcccagctaatttttttttttttttttgtagagacgcgatcttcctatgttacccaggctggtcttgaactcttgagctcaagcagtcctgccttggcctcccaaagtactgagattacaagcatgag

SEQ ID NO: 2 - DNA, 14881 bp, Mus musculus
tggacgtctgctaagggaccccgacaaggcctagcttgaataagtgggtacacttgtagaggcaggagattgaaacccaggaaggactgggctaggagtctggctgactgtaggaaagggagctcaggcttcaggagaggctgtaagggagaggaaaggtgagactacgaggaatggtggaagtttcgggtatctctctgcccctctcaccagtgctcccctcaaagcctgatgtagcccagggagacgaagccggagaaaatccatcctgcaggtctctgtgtgtccagaaagctcacaatgaccatccgtccccacccacgaagtcctccccagcacccaagacacttcctcttcccctgactctccctgacaaatctccaccccaggccctgtgcacctcccccctgctcatggatgacttcatgacaggcagggggatggcttaacagtgtccaggtgactcagactcgggtgcacatctggaactaaagggcagaggatcgggccagaaaggggaaagggaaaaaaaaagtggaaacaggagacctcagaattgggtatgggggagggggcattcggcaagtgcagcctgagtgactcaggtcttgtattcgcacctcagccatttccgcctcctccaagccacagccttcagtgagaggaagctagggtttgtggttcctgatttatttgggggagaagctgtagaagtgaggataaggccactggaagttctgggttcccattttcaacgccattaagatttttctggggatgaaaggtctggattggtgctcaatgcccacaccctcacctatggttattcttgtactgtggtcatgtgtcctcacagcttactgtgagttccttaaggacagaggctgcagaatttctagagactcgtttgggtaggaggatcctttttctcactgtttgagtgatctaggaagaacaccacaggagggcatatttcaaacggcaaatcttaatgacaggtggttgaaaagacccgggagaagtgggcagaggctgctggaatccatagcagaggaataggcttgatgaagaatggtcaagatgctgccctgccatcgagtggaaggtaagggaaggactccttatttggaactgtgaggtgacacattccaactatcctctcagcctcattctcctaacttccaattaggacaaacggctgacatcacagaccaggaaaaacacccatagcagctttctcagtgaacttagcacctttgctctatttcttactgctgagtctctgaagtcagctaaaagtctagcctctgttgcacagtggggcggagggaccttggttttctcttgtaaaaccaggggttggacaactttcgtggtatttgttgttctaggatagtaaggcaaaggggatttaaccgtaacatcgtgaatattgccacagataattcatagaatgctgttttgtgggtgtttatttttaatcgatctactctcagctacttcccagatgcttattataggtctttacaataggtctgtaaggacaacagataacacttgaggctggtcgcctttgcgctcaagcagaacttgtccttgggacgttgggtggggtagtgggacatccgcagcaaagtagccataattgtttcaggcaagtaagagctgcaggaaagaagctgaagccttccgcgccatgactagtactggcagatttggtcggcgtctagagcgccgcttttcatggctctacttgtggctcccttcgactccaattcaaaatcagtgcttccagcgttctctcgaactctccttcttttattctccgaagtttgagaagtccaaaggcgaaaaagagaaaaaaggacccggaagtaagaagcaaccctcaagcacgtgatggaaggctcacgggtactgtcacgtgacccccaacctgctcgtctaccttgcggagagttcagctgctcttaaaggtacaggcctgggggcgggggctttgctttagaggggcagggggcggggtcaggcgggtggggcgtgggtgaacacgagctggagatctggggatccttcgacggacatgtcgtgtatagcagacagcggaacctgggactgaccgcggggcattgatccttcgcacccacctgtcccagactttaatctgttttcttgaagctagctcggaacacacgctgactttgggccctttgggggacccgaactcaatgttatgggaagttctgcgggctcgtggaggcgccttgaggattctgagagtgagtgctctcatcggttgccttgggaatgaccacctctgcccgcggctctcccgaggagttgggatgctggccctgccccctcaacaccatgcgggataggcggggccttcgtgtacccgagtgagtggggggtcatgctttctctacctcccaggggaggagaccgactcagagccccaggcccctcggttggatagtcggagtgtcctttggaagaatgcagtgggtttctggtaagtggtctgagagttgtgcaaaagtaaaatgccagctgggcgtggtggcacacgcctttaatcccagcatttgggaggcagaggcaggcggatttctgagttcgaggccagcctggtctacagagtgagttccaggacagccagggctacacagagaaaccctgtctcgaaaaacaaacaaacaaacaaacaaacaaagtaaagtgcgattggagcaaagatgagagccaagttgtgaaaagcagttgaatacacagatacttaatcaactgtgaactttccaagttccattaaattcccaccttcacctattgatcagatcttagtatctgtagagttatttattaaatgttttcctccagctaactttgttttttttcctcttccttcctttctttctttctttctttcttcctcttcctcctcctcctcttcctcttctttttcttcttttttttggaaaggtctttaaaccctacaagcagtaagggcttactttgacctttctaatcctgcttttgccttccaggtgctgcagttacaggccctactgtagcacaatgcccagaacacccattgtttctgtttgcagtgcagggtattaaaagcaggtccttcccgggctcacgcactgttgtacaagtgagctctgtctccagccaatttgcttttgccaagttctgagcacagctctgggagatgaaaataagaacaatatgccctgctattattttggttcccttgagatcagcaggtgaaaatttgtctccgttttgcacaggaatacgctagatatttgcattgtaccacagtgtttccgttgatgagaaatgcccagggatcagaatcttagtgtaagcgtgggagaaatagtgcttaggaggacttggcacttggccatgagctggtctaatctcaaggcctgttttgatagtggagaaggaaaaaaaaaaggtgtatctttaaggactgtaagtggatagaaagccagacagtgaagatgtgtcctcatttctaggatcttgggtctttgcaacaatttctcatatgtggtgatgctgagcgctgcccatgacatcctcaagcaggagcaggcgtctggaaaccagagccatgtaagtaagcccctcttcaccaccaccataggtcagtaagtgagcccctctgtaccactaccataggtcagtccgcatggggaatactaagaacccactcagggccagaggacccctgaaaaactcctagagtagctctgggtgggattggggagctaaaggtcttagtctcccacatcctcatggcaggagggatcttaaagagagcaacgggtaccaggttggtgaactcacataactggctgggtaagaccttattttgtggtgtgttttgtcattgtaccctgttgacatttgaggtcaggtgactgttgtgagtctgtcctgcatgttgtagatgtttagtagcatcccatgccccttgtcaaccaaaagtgacagccaaaaatgtctccagatgttacaaatgctcctgccaggtaacattacccaggtggagagcctttggcggcagtgagctagagcacgcaggccctaagaggcgcagcctccactttgcaggttcactaggaaatttgacccaagctgatattttccagaaacccagatttctatatgaatgctcttaaacatggtgcatccatcttggggggctgtctttggcctctggcccaaagtttgagatgcttataaacactctcttcattgtggacattgaaaaactaaggccccctagaagccttgcctacccacagccatttgtgaagttaggacagaactggacctgggccttggttagcctcctgccgctttgtcggaaagaccctcttctctcccataggtggaaccaggcccaacacccacaccccacaacagctcatctcgatttgactgcaactccatctccacagctgtgagtgtccatgtggggaccccacgatccttgcctttgcaatctcgtttccccacccccacccccatccagttgggaatgttgggattgcagtgatgaatataaatatgtaaatagtaattattagttcaggtgccccccttcccccagctttgtgagtgaaacactgtaagtttccctctttggatgaggaaactgaaggtcagagtgtataaatatgccctgacagccatttgaggagccccagagcagggaatgaatggagatctatctaactccagagcctaacttacagcaggtctcactggccggctccccggctgggttgggaaggttgtctccgggaaggtctaggctctcactgtgttctcctgtctcaggcggtgctcctagcagacatccttcccacccttgtcatcaaactcctggcgcctcttggccttcacttgctgccttacaggtctgggtcggggtggcaggagggaggcagggtgggaggaagcgggagtctggagagtctcgcatggaggtggccttctgagttcctcctcatctcttctcagcccccgggtgctcgtcagtggagtttgttctgctgggagctttgttctggttgccttctctcagtcagtggggttaagcctgtgtggtgagtacgaagctgggagaggttgatggactcaacagagtgatgtgctgtagtctccccagcttcctgtgtgtgtgtgtgtgtgtgtgtgtgtgtgtgtgtgtgtgtgtgtgtgagagagagagagagagagagagagagagacacacacttgggagattgtgaatttgagattagttggggccacatagtgaattcaagcctatcctaagctgcacagcaaaaccctatctcaaaaaacaaagacaaaaaaaaaccaaaaaaacaaggagagggccagtccaggctgatttacatagcaagttccaggccagcctggactacatccccgagactttgtctcaaagacaaacctcctagagcttccctaaccagaagtgagctctaggaacaattggagggtgtcaataggaaggaatggggagactgagtataggtggggatgagcagggttgaataattgggaattattaactgaatgaagatgggggtacagtctcggagaggacatgtggaggagggtgtggcttttcagatgagaccctagcctgtctgttcagtgacagatctgttcttagccctgctgagctgatggagttgaggctgtaaacaaaccaccggggactgcggctttcagttaaaggtcactgtttgctgtttgtcctcccaggagtggttttggccagcatctcctcagggctaggggaggtcaccttcctctcactgactgccttctaccccaggtaagcggcctgggccaggaggactgggagacgagtgccccctgctggcttccatcttcctcagtgctcttgtcttcttttcagtgctgtgatctcatggtggtcttcgggtaccgggggtgcagggcttcttggatcgctgtcttacctgggactcacccaggctggcctctccccgcagcacaccctactttctatgttggggatccctgttctgctgctagccaggtgagtgtccttaggcccaaaggatgaaagatggaaggaggagcttgatgctaatccaatcttcagagctcttcagccttgttgtgaaggggtgtggactctggactctcgacacccctcaggtggaatctcagagtggggctgtgttagctgtgcgactctggacaacttcattcctgaccttcagttgtctctgtctcaggctggttctgtctcatccgcacaggtcggctcctgagactggggctgggactgagtgcaccccggtttatttcttctgtgtgtatttgtatattttagaaaacattattaagccaggtgtagtggtacatgcctttcatcccagcactctggaggcatcatgttatagcttagggctattgtggggtatccaccagaaagacggctcagacatgggtttaaacccagtggaaagccgattagtcacctggtaactacacactgcatgttcagtggcatttccattggagtcagttgtcctaaggtccggtggcctgttacaaggctatcttgaagaaaaggagtcagtatgtgtgtaatgctgaacacccacagtcctggctcttgggaggtgatggaaaaggctcatgtgtttgaagcctacctgggatacacgtggagaccctggctcaaaagctaactgagataattgaacatatggcaaatgcctctaatccaagccctcaggaggcaggaggatctctgtgagtttggggtcagcctggtctacatcttgagctccaggccagccagagctgcatagttaagaccctgtctcaaaaaaaaaaaaaaagaaagaaagaaataaagcaatcccatgatcatacagaccctgggtaaactcagtcacaacacgaaacaaaaagacactgagtatgagaaagggacctgtgatgtgggaaaggtcacagggtgtggggacccgagggggtggggagagtcaccagagggcagtatatatgtgaataaaaatgtcaagaaacacatttagtaagaatctctgtgaagtggtgaatagatgagaagtactgagaacagggaaacgagcaggtgctgtgactgagtcaaatgaggtccctgggtagaaacaaagaagcttttccagcacagggtacctagaggctgggagcaccccatgcaagtgtgaagggcctggaggggcgggcctgcaggacctctggggcctttcccgacaagagccccctgactcgcttctccattccatcccagctatttcttgttgctcacgtctcctgaacccctggaccctggaggggaaaacgaggcagagactgctgcccggcagcctctcataggcaccgagaccccagagtcaaagccaggtaggatgcaaaggccctcccatcccagggccactttccgccttggggttttggtctcaacagtggacacagttgagggaaacaggctgctaggtgtacaggtctgggtcaaagcagtcatgtatacttactaactactgactggatgccagacattccgattgctgggaatggtagtgctccagatgaaggaatgagagcattttcacaggacataaaactgcgatggggcccagaggggtggcgcacaccttgaatcccaacactccggaggcagccacaatcagatctctatgatttcaaggccggttgggctacatagtaagaccctgcacagacccagaaagtaacggttaaggaaatggggtggggatggctaagggagctgctttacaggaggttccaagtgtgggggttttgttgttgttttgttttttcttcaagtttttgcttattggtttttaaatagtctctctgtatagccctagctggcctagaaatcactgtgtactctcgaatggcctcaacctcacaaatccacctgcctctgcctctcaaatgctaggagtaaacgcatatgccaccatacacctggctaggatttatttttctatttattgatgtgtatgtgtatttgtgaacttataccgcggatgaccagcagagggcatcagatgccttggagttgcagttacaggcagttgggagctggccgatgttgccaggattcaaacgtggaccccctcatggttgagcaaccagtgctcgctcgctctttctttccttttctctctttctttctttctttctttctttctttctttctttctttctttctttctttctttctttctttctttctttcttccttccttccttccttcctttctttctttctttctttcggatttatttatttatttaatgtatatgagcacaatgtagttgtcttcagacacaccagaagagggcaagagggcatcagatctcataacagatggttgtgagccaccatgtggttgctgggaattgaactcaggacctctggaagagcagtcagtgctcttaaccactgagccatctctccagcccctctttcttttttttttattgtttgtttgttttgtttttgttttgttttttgtttttttgttgttgttgttgaggcagagcttctctgtgtagctctggctgtcctggaactcactctctagaccagaagtcaaaaattctcctgcctctgctttccatgcgctgggattcagggcgtgtgccgccacttaaccccaagccacctctccaactctgagaaaggctctacttatttatttaattttgtaattatttgtttgtttgtttgcttgcttttcgaggaagggtttctctgtgtagccctggctgtcctggaactcacactgtatgtaatccaggctgccctcaaactttcagagatccacctgcctcagcctcccaagtggggattaaaggcatgcgccaccactgcccagcgatttgtctatttttattttatgtgcattggtgttttgcctgtgtgcatgtctatgggagggtgttggatcccctggaactggagttacagacagttgtgagctgccacgtgggtgctgggaattgaggtccgctggaagagcagccattactcttaaccactgagccatctcttgagcccccgagagaacccccaacaacaaaccttttatttttgagaatggagctgtctgggcaagagcgtgtgcacaagtgtgaggtctgacgttctgaggattgagctctgctcattcagactcggcagtgagtgccctcatccaccgagcatccatcagcccaagacagccttttgaaggatatctctgggctgaaacttggctgtcaaggtgtcagctttgcaaatgcctaggtgatcatgttccaggcacaggaaacagctcagggtgaggccttatgtgagaaaaggagcctggttgttctagaaatgaaaggcttagccagagtctgggatggagtcggtagataaaagctgtaccctgatctatagtcttgctcattaaaaaaaaaaaaaaaaagttttataagtatatgtgtgtatatacatatgtatacagcgcgaagtttaccattttagcgactactaagtgtgcactaagtggcatttaaatccacacactgctgtgctggctggttgcttgctgcatgctggcaccttagctaagcgttcagcgtgcaacctcctccccttccccctgctctgtaggtgccagctgggacctctccctccaggaaaggtggacagtgttcaaggtttgaatggtggctgcgggtgccccgtgaggatcctctgcctcttgccttccacttacatctcccgactcttccagggtctcttgtggtacatcatccctctggtgctggtctactttgcagaatactttatcaaccagggacttgtgagtgaggggcgccggggagggatgtgggaggagagatagggctgaggttccgccatctccatctgtctggtcttcgctgcagttcgagctcctgtttttccggaacacttccctaagccatgctcagcagtaccgatggtgagagaagttggcaggtgggcagtaggctgggaggatccctgatgttggggtcctggggttgaccccaaaaagtgccctgagagcctcttcctcttctctgccccaaccttaggtaccagatgctataccaggctggtgtgttcgcctcccgctcttctctccaatgttgccgaatacggttcacctgggtcctagccctgctccaggtactgagtgctgccctgtttgttcacacccacctcggcctcctgagtttcttacattagtgtactccagcgttcctaagtacagcactaaaggccagctggactccatataaaatgtgtggtggcaccaggaggaaccagtcatacttgtgggtaaattccagtttctgttctcaagagctgtgtgactcagagcagtctctgaacctctattgagcctcagttttcctatcaggaagtcagatggggtggagcaggattgtgcacctagcctgtctgaggctgtgggtcccatccctagtactgcaaaaattaaaatatttaatagctgtgggcaacacctatttctcagtgtttgtatagcaatgttgagaaaaaaaaggaagaacaagatattgttttgatttattttggttttgttcttgtctgctttatttttttttctttgtttgtttttaaggtagggtctcactgtgtagtcctgggtggcctggaatccgatatgtagactaggctagcctgaagctcacagatctgcttcatgaatgcgggcgttgagggcctgtgcctcgtgcctggccgtcgtgctttgttctcacagtcgttctgactgcacctggggccttgttcctgctggtccatgctgattgttacgaaacttcttaagccctcaagcttgctttcatttacttacttatttactttaaagatttgtttgtttatatataaacaaactgtagctgtgttcagacacaccagaagagggcatcagctagccgggcgtggtggcgcacgcctttaatcccagcactcgggaggcagaggcaggcggatttctgagttcgaggccagcctggtctgaaaagtgagctccaggacagccagggctacacagagaaaccctgtctcgaacccccccccaaaaaaaaaaaacaaaaaaaaacaaaaaaaaaaaacagaagagggcatcagatcccatcatagatggttgtgagccaccatgtggttgctgggaatcgaactcaggacctctggcagagcagtcagtgctcttaaccgctgagccatcgctctagccctatttatttattttatacaatttaaataaacacacttatatttatatttatttattggtgggagggagacacttgccaagccgtgtgtatggaggtcagaggacagcgtgcaggattggttcccttcttcaacgtgtgggttctggtgatcaaactcaggtcatacaggcaagtacctgtagctgagccatctcttcagcccaaacagggtttctctgtatagccctggctgtcctggaactcactctgtggaccaggctggcctcgaactcagaaatctgcctgcctctgcctcccaagtgctgggtttaaaggcgtgcgccaccactgcccagctcagcccaaactttttatttggaggcaaagtcttcaaagctggccttgaacctacatctgaggcccacataagccttgaccattgtttagtttatatatacacaaacctttgtctatgtgtatatatgtgaaccacctgtgtgcctggtgcccatggaggccagagagggtatcagatccctggaactggaactacagataggtgtggaccgccctgtgggtgctgtggaagaacagcgggtgcctttaactactgagccatctttccagtccaaaattttgtactcttgacatttgcagctagatcattctgatgtgttacttgctgtaggtgtttagcagtatccctagctggtgagacggcaagtaggtctccagacattgtcaaatgacccattgatttcagtgcagtaattaggggagaacaggtgatgttcagggggtccttgggtgctgaggataggactctggtagagcatttgcctctgaatcacaagctctcgtgttagctctgcagtgtatgtgtgtgtgtgtgtgtgtgtgtgtgtgtgtgtgtgtgtgtgtgtttgtgtgtgtgatacacatggtaggggtgtctaggtgttaggattcatgccattaaaaaagaaacaaaaaggcaagaaggagaggaggtgactgatgaccacagtggaatgaccctggtccaactgagagctcaccagtgatgggaagttgaggaacattacaggctaagagtgacagggacaagtcaaggaaggggagaaacccacaaggcatgtgaggatgaggatggggtcatcgagggcctaccaagatgtcggggtcccacaggcccagctgggcactgggaccctacacagccctctgcttcagcggcatctcctataacctggcagctctgagacctgctgcctccctgccgtattgttgcccagtctcacgcctcccttccctctgcccacagtgcctcaacctggccctcctgctggcagatgtctgcttgaacttcttgcccagcatctacctcatcttcatcatcattctgtacgaagggctcctgggtggggccgcttacgtgaataccttccacaacattgctctggaggtctgtgccagttggacaagggctgtgggtggccttaggagccttgtggatggaagccctaacccttgttctttgctctcccagaccagtgacaagcaccgagagtttgccatggaagctgcctgtatctctgacaccttgggaatctccctgtcgggggtcctggccctgcctctgcatgacttcctctgtcacctcccttgacaggagttgctcgacacacactgatctgcaggcacatgagcagatcacacatcttcgagctctgccacagcctttccctgccccactgcagcaaggagcccctgatgtttcccactcctgagctggcctcagagttttctcctaccctctgcccttctaataaatgcttattttaacagtttcctttttgtgcagtctctgaaacggaaagtttacataagcccactgtgaaagatatatgtatttatgaatgtataaagtatttcagacaaggtgtggcacacacctgtaattccagcactcagcagactggagaatccaagttcacgtctgactgggatatacggtgagaccctgaggactagggctatagttcagtggtagagtacttgcccaggatgtcctgggctctcagttttatccctggaaaacaattcaaaagcagcgtatgctaatataagcctgtagtcccaggattggggaggtagagacaggaggatccagagttcatggtcctcagctacataggaaaccaaggtcagcctggcctacataagatcttgttttacaataattacattgtgtatttctggcttctttataaaaaaaaatcaaatgtccataggtatgtagatttatatctgggtcttcgattccattgatcaacttgtctgtttttatgccaatactgtgtgggttttattactataactctgtagtagtgcttgaaataaggatggtgatacctccaggagttctattactgtacagagttgttttagcaatcctgtgtttcttgtttttttcatatgaaattgagtattgttctttcaaggtctataaagaattgtgttggaattttgatggacaactgatttctttcttcaatgtcttaaagtttttatcatataactctttgacttgcttggttagattgaccctacaatattcatagaagacgaagtgggtagtagccctgaacgaatgtcacaggagacaacttcctgaacagaacaccaatagttcaggcactaaaattgacaataaaccagatctcatgaaactgaaaagcttttgtgaggcaaatgatattgtcatctggcagcctacagaatagaaaaagatttctaccaactcaatatctgatagaaggctaatatccgtaatatataaagaactcaaagccgggcggtggtggcgcacgcctttaatcctagcactcaggaggcagaggcaggtggatttctgagttcaaggccagcctggtctacaaagtgagttccaggacagccagagctacacagagaaaccctgtctcgaaaaaaaaacaaagaacaaaaaacacgagggagagagaaagagaaagagaactcaagaagctagacatcaataaggcaaatagcccaaataaaaaatggggtacagagctgaacagagacttctcaacaggaatctcaaatggctgagaaacacttagaacttcatcatccttatccagcagggaaatgcaaatcaaaaccactctgagattccatctttaacacctgtcaggatggctaagatcaaaactcaagtaacagctcatgctggcgaggatgtggagaaaggggagcacttgggagcaaggggagcactcttcggttgttggtgagaatgcaaatttgtacagccactttggagatcaatatggtagtttcttagaaaattggaacctccagacccagctataccacttctggggatatacccaaaggatgtcccatcctaccacagggacacttgctcaattatgttcacagcagctttatttataataaccagaaactggaaacaacctcgatgtcccttaactaaggaatggataagaaa

SEQ ID NO: 3 - DNA, 18 bp, Artificial sequence
  Synthetic oligonucleotide
acaaccttcccaacccag

SEQ ID NO: 4 - DNA, 18 bp, Artificial sequence
  Synthetic oligonucleotide
cggagacaaccttcccaa

SEQ ID NO: 5 - DNA, 18 bp, Artificial sequence
  Synthetic oligonucleotide
cttcccggagacaacctt

SEQ ID NO: 6 - DNA, 18 bp, Artificial sequence
  Synthetic oligonucleotide
tagaccttcccggagaca

SEQ ID NO: 7 - DNA, 18 bp, Artificial sequence
  Synthetic oligonucleotide
gagcctagaccttcccgg

SEQ ID NO: 8 - DNA, 18 bp, Artificial sequence
  Synthetic oligonucleotide
agtgagagcctagacctt

SEQ ID NO: 9 - DNA, 18 bp, Artificial sequence
  Synthetic oligonucleotide
aacacagtgagagcctag

SEQ ID NO: 10 - DNA, 18 bp, Artificial sequence
  Synthetic oligonucleotide
aggagaacacagtgagag

SEQ ID NO: 11 - DNA, 18 bp, Artificial sequence
  Synthetic oligonucleotide
gagacaggagaacacagt

SEQ ID NO: 12 - DNA, 18 bp, Artificial sequence
  Synthetic oligonucleotide
cgcctgagacaggagaac

SEQ ID NO: 13 - DNA, 18 bp, Artificial sequence
  Synthetic oligonucleotide
agcaccgcctgagacagg

SEQ ID NO: 14 - DNA, 18 bp, Artificial sequence
  Synthetic oligonucleotide
ctaggagcaccgcctgag

SEQ ID NO: 15 - DNA, 18 bp, Artificial sequence
  Synthetic oligonucleotide
gtctgctaggagcaccgc

SEQ ID NO: 16 - DNA, 18 bp, Artificial sequence
  Synthetic oligonucleotide
aggatgtctgctaggagc

SEQ ID NO: 17 - DNA, 18 bp, Artificial sequence
  Synthetic oligonucleotide
tgggaaggatgtctgcta

SEQ ID NO: 18 - DNA, 18 bp, Artificial sequence
  Synthetic oligonucleotide
aagggtgggaaggatgtc

SEQ ID NO: 19 - DNA, 18 bp, Artificial sequence
  Synthetic oligonucleotide
atgacaagggtgggaagg

SEQ ID NO: 20 - DNA, 18 bp, Artificial sequence
  Synthetic oligonucleotide
gtttgatgacaagggtgg

SEQ ID NO: 21 - DNA, 18 bp, Artificial sequence
  Synthetic oligonucleotide
caggagtttgatgacaag

SEQ ID NO: 22 - DNA, 18 bp, Artificial sequence
  Synthetic oligonucleotide
ggcgccaggagtttgatg

SEQ ID NO: 23 - DNA, 18 bp, Artificial sequence
  Synthetic oligonucleotide
caagaggcgccaggagtt

SEQ ID NO: 24 - DNA, 18 bp, Artificial sequence
  Synthetic oligonucleotide
aaggccaagaggcgccag

SEQ ID NO: 25 - DNA, 18 bp, Artificial sequence
  Synthetic oligonucleotide
aagtgaaggccaagaggc

SEQ ID NO: 26 - DNA, 18 bp, Artificial sequence
  Synthetic oligonucleotide
gcagcaagtgaaggccaa

SEQ ID NO: 27 - DNA, 18 bp, Artificial sequence
  Synthetic oligonucleotide
gtaaggcagcaagtgaag

SEQ ID NO: 28 - DNA, 18 bp, Artificial sequence
  Synthetic oligonucleotide
gacctgtaaggcagcaag

SEQ ID NO: 29 - DNA, 18 bp, Artificial sequence
  Synthetic oligonucleotide
acccagacctgtaaggca

SEQ ID NO: 30 - DNA, 18 bp, Artificial sequence
  Synthetic oligonucleotide
ccccgacccagacctgta

SEQ ID NO: 31 - DNA, 18 bp, Artificial sequence
  Synthetic oligonucleotide
tgccaccccgacccagac

SEQ ID NO: 32 - DNA, 18 bp, Artificial sequence
  Synthetic oligonucleotide
cctcctgccaccccgacc

SEQ ID NO: 33 - DNA, 18 bp, Artificial sequence
  Synthetic oligonucleotide
gcctccctcctgccaccc

SEQ ID NO: 34 - DNA, 18 bp, Artificial sequence
  Synthetic oligonucleotide
accctgcctccctcctgc

SEQ ID NO: 35 - DNA, 18 bp, Artificial sequence
  Synthetic oligonucleotide
ctcccaccctgcctccct

SEQ ID NO: 36 - DNA, 18 bp, Artificial sequence
  Synthetic oligonucleotide
gctaggagcaccgcctga

SEQ ID NO: 37 - DNA, 18 bp, Artificial sequence
  Synthetic oligonucleotide
tgctaggagcaccgcctg

SEQ ID NO: 38 - DNA, 18 bp, Artificial sequence
  Synthetic oligonucleotide
ctgctaggagcaccgcct

SEQ ID NO: 39 - DNA, 18 bp, Artificial sequence
  Synthetic oligonucleotide
tctgctaggagcaccgcc

SEQ ID NO: 40 - DNA, 18 bp, Artificial sequence
  Synthetic oligonucleotide
tgtctgctaggagcaccg

SEQ ID NO: 41 - DNA, 18 bp, Artificial sequence
  Synthetic oligonucleotide
atgtctgctaggagcacc

SEQ ID NO: 42 - DNA, 18 bp, Artificial sequence
  Synthetic oligonucleotide
gatgtctgctaggagcac

SEQ ID NO: 43 - DNA, 18 bp, Artificial sequence
  Synthetic oligonucleotide
ggatgtctgctaggagca

SEQ ID NO: 44 - DNA, 18 bp, Artificial sequence
  Synthetic oligonucleotide
tgtaaggcagcaagtgaa

SEQ ID NO: 45 - DNA, 18 bp, Artificial sequence
  Synthetic oligonucleotide
ctgtaaggcagcaagtga

SEQ ID NO: 46 - DNA, 18 bp, Artificial sequence
  Synthetic oligonucleotide
cctgtaaggcagcaagtg

SEQ ID NO: 47 - DNA, 18 bp, Artificial sequence
  Synthetic oligonucleotide
acctgtaaggcagcaagt

SEQ ID NO: 48 - DNA, 18 bp, Artificial sequence
  Synthetic oligonucleotide
agacctgtaaggcagcaa

SEQ ID NO: 49 - DNA, 18 bp, Artificial sequence
  Synthetic oligonucleotide
cagacctgtaaggcagca

SEQ ID NO: 50 - DNA, 18 bp, Artificial sequence
  Synthetic oligonucleotide
ccagacctgtaaggcagc

SEQ ID NO: 51 - DNA, 18 bp, Artificial sequence
  Synthetic oligonucleotide
cccagacctgtaaggcag

SEQ ID NO: 52 - DNA, 19 bp, Artificial sequence
  primer
gcaactctgtctctacggc

SEQ ID NO: 53 - DNA, 17 bp, Artificial sequence
  primer
cttgaacactgtccacc

SEQ ID NO: 54 - DNA, 18 bp, Artificial sequence
  primer
caactccatctccacagc

SEQ ID NO: 55 - DNA, 18 bp, Artificial sequence
  primer
agaggtcccagctggcac

SEQ ID NO: 56 - DNA, 18 bp, Artificial sequence
  primer
gcctcaggagatgtgagc

SEQ ID NO: 57 - DNA, 18 bp, Artificial sequence
  Synthetic oligonucleotide
acaaccctcccaaccacg

SEQ ID NO: 58 - DNA, 18 bp, Artificial sequence
  Synthetic oligonucleotide
gggacaaccctcccaacc

SEQ ID NO: 59 - DNA, 18 bp, Artificial sequence
  Synthetic oligonucleotide
aggggacaaccctcccaa

SEQ ID NO: 60 - DNA, 18 bp, Artificial sequence
  Synthetic oligonucleotide
cttccaggggacaaccct

SEQ ID NO: 61 - DNA, 18 bp, Artificial sequence
  Synthetic oligonucleotide
cagagcttccaggggaca

SEQ ID NO: 62 - DNA, 18 bp, Artificial sequence
  Synthetic oligonucleotide
gaccgcagagcttccagg

SEQ ID NO: 63 - DNA, 18 bp, Artificial sequence
  Synthetic oligonucleotide
agtgagaccgcagagctt

SEQ ID NO: 64 - DNA, 18 bp, Artificial sequence
  Synthetic oligonucleotide
aatagagtgagaccgcag

SEQ ID NO: 65 - DNA, 18 bp, Artificial sequence
  Synthetic oligonucleotide
aggagaatagagtgagac

SEQ ID NO: 66 - DNA, 18 bp, Artificial sequence
  Synthetic oligonucleotide
gggacaggagaatagagt

SEQ ID NO: 67 - DNA, 18 bp, Artificial sequence
  Synthetic oligonucleotide
agcctgggacaggagaat

SEQ ID NO: 68 - DNA, 18 bp, Artificial sequence
  Synthetic oligonucleotide
agcacagcctgggacagg

SEQ ID NO: 69 - DNA, 18 bp, Artificial sequence
  Synthetic oligonucleotide
ccaggagcacagcctggg

SEQ ID NO: 70 - DNA, 18 bp, Artificial sequence
  Synthetic oligonucleotide
gtccgccaggagcacagc

SEQ ID NO: 71 - DNA, 18 bp, Artificial sequence
  Synthetic oligonucleotide
aggatgtccgccaggagc

SEQ ID NO: 72 - DNA, 18 bp, Artificial sequence
  Synthetic oligonucleotide
gggaggatgtccgccagg

SEQ ID NO: 73 - DNA, 18 bp, Artificial sequence
  Synthetic oligonucleotide
tggggaggatgtccgcca

SEQ ID NO: 74 - DNA, 18 bp, Artificial sequence
  Synthetic oligonucleotide
gagtgtggggaggatgtc

SEQ ID NO: 75 - DNA, 18 bp, Artificial sequence
  Synthetic oligonucleotide
atgacgagtgtggggagg

SEQ ID NO: 76 - DNA, 18 bp, Artificial sequence
  Synthetic oligonucleotide
atttgatgacgagtgtgg

SEQ ID NO: 77 - DNA, 18 bp, Artificial sequence
  Synthetic oligonucleotide
caacaatttgatgacgag

SEQ ID NO: 78 - DNA, 18 bp, Artificial sequence
  Synthetic oligonucleotide
ggagccaacaatttgatg

SEQ ID NO: 79 - DNA, 18 bp, Artificial sequence
  Synthetic oligonucleotide
caagaggagccaacaatt

SEQ ID NO: 80 - DNA, 18 bp, Artificial sequence
  Synthetic oligonucleotide
aaggccaagaggagccaa

SEQ ID NO: 81 - DNA, 18 bp, Artificial sequence
  Synthetic oligonucleotide
aggtgaaggccaagagga

SEQ ID NO: 82 - DNA, 18 bp, Artificial sequence
  Synthetic oligonucleotide
gcagcaggtgaaggccaa

SEQ ID NO: 83 - DNA, 18 bp, Artificial sequence
  Synthetic oligonucleotide
gtagggcagcaggtgaag

SEQ ID NO: 84 - DNA, 18 bp, Artificial sequence
  Synthetic oligonucleotide
gacctgtagggcagcagg

SEQ ID NO: 85 - DNA, 18 bp, Artificial sequence
  Synthetic oligonucleotide
acccagacctgtagggca

SEQ ID NO: 86 - DNA, 18 bp, Artificial sequence
  Synthetic oligonucleotide
ccctcacccagacctgta

SEQ ID NO: 87 - DNA, 18 bp, Artificial sequence
  Synthetic oligonucleotide
cactaccctcacccagac

SEQ ID NO: 88 - DNA, 18 bp, Artificial sequence
  Synthetic oligonucleotide
cctcccactaccctcacc

SEQ ID NO: 89 - DNA, 18 bp, Artificial sequence
  Synthetic oligonucleotide
ccctgcctcccactaccc

SEQ ID NO: 90 - DNA, 18 bp, Artificial sequence
  Synthetic oligonucleotide
gcccaccctgcctcccac

SEQ ID NO: 91 - DNA, 18 bp, Artificial sequence
  Synthetic oligonucleotide
ctcctgcccaccctgcct

SEQ ID NO: 92 - DNA, 18 bp, Artificial sequence
  Synthetic oligonucleotide
ctcagctcctgcccaccc

SEQ ID NO: 93 - DNA, 18 bp, Artificial sequence
  Synthetic oligonucleotide
cctttctcagctcctgcc

SEQ ID NO: 94 - DNA, 18 bp, Artificial sequence
  Synthetic oligonucleotide
cctcccctttctcagctc

SEQ ID NO: 95 - DNA, 18 bp, Artificial sequence
  Synthetic oligonucleotide
cccagcctcccctttctc

SEQ ID NO: 96 - DNA, 18 bp, Artificial sequence
  Synthetic oligonucleotide
gccatcccagcctcccct

SEQ ID NO: 97 - DNA, 18 bp, Artificial sequence
  Synthetic oligonucleotide
ttagtttaatcacgctcg

SEQ ID NO: 98 - DNA, 203 bp, Homo sapiens
cgtggttgggagggttgtcccctggaagctctgcggtctcactctattctcctgtcccaggctgtgctcctggcggacatcctccccacactcgtcatcaaattgttggctcctcttggccttcacctgctgccctacaggtctgggtgagggtagtgggaggcagggtgggcaggagctgagaaaggggaggctgggatggc

SEQ ID NO: 99 - DNA, 1915 bp, Homo sapiens
ataactggtgctggcaggctactgtctcggtcttgggcgccactgatctaaggtcacggctctgcttgctgctcccacccgctccagtttaaaacctgcggttccagggttctccagcccctccctttttcacgctccgaagccgagaaggcccaaagcgaagacagagaggacccggaagtagggaaaacctctgagcacgtgatgggggaacacgcgggtgctgtcacgtgatccgacaaacggcctctgcatagtgcagaacattctgctgctcttaaagaccctcatccctcccgtgggagccccctttggacactctatgaccctggaccctcgggggacctgaacttgatgcgatgggaggctgtgcaggctcgcggcggcgcttttcggattccgagggggaggagaccgtcccggagccccggctccctctgttggaccatcagggcgcgcattggaagaacgcggtgggcttctggctgctgggcctttgcaacaacttctcttatgtggtgatgctgagtgccgcccacgacatccttagccacaagaggacatcgggaaaccagagccatgtggacccaggcccaacgccgatcccccacaacagctcatcacgatttgactgcaactctgtctctacggctgctgtgctcctggcggacatcctccccacactcgtcatcaaattgttggctcctcttggccttcacctgctgccctacagcccccgggttctcgtcagtgggatttgtgctgctggaagcttcgtcctggttgccttttctcattctgtggggaccagcctgtgtggtgtggtcttcgctagcatctcatcaggccttggggaggtcaccttcctctccctcactgccttctaccccagggccgtgatctcctggtggtcctcagggactgggggagctgggctgctgggggccctgtcctacctgggcctcacccaggccggcctctcccctcagcagaccctgctgtccatgctgggtatccctgccctgctgctggccagctatttcttgttgctcacatctcctgaggcccaggaccctggaggggaagaagaagcagagagcgcagcccggcagcccctcataagaaccgaggccccggagtcgaagccaggctccagctccagcctctcccttcgggaaaggtggacagtgttcaagggtctgctgtggtacattgttcccttggtcgtagtttactttgccgagtatttcattaaccagggactttttgaactcctctttttctggaacacttccctgagtcacgctcagcaataccgctggtaccagatgctgtaccaggctggcgtctttgcctcccgctcttctctccgctgctgtcgcatccgtttcacctgggccctggccctgctgcagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggagaccagtgatgagcaccgggagtttgcaatggcggccacctgcatctctgacacactggggatctccctgtcggggctcctggctttgcctctgcatgacttcctctgccagctctcctgatactcgggatcctcaggacgcaggtcacattcacctgtgggcagagggacaggtcagacacccaggcccaccccagagaccctccatgaactgtgctcccagccttcccggcaggtctgggagtagggaagggctgaagccttgtttcccttgcaggggggccagccattgtctcccacttggggagtttcttcctggcatcatgccttctgaataaatgccgattttatccatggaaa

SEQ ID NO: 100 - DNA, 1879 bp, Homo sapiens
gtgctgtcacgtgatccgacaaacggcctctgcatagtgcagaacattctgctgctcttaaaggtacaggcctcagggtccctgctgtagacggggcgggggagagtacgatgggtggggcgtggtgggtcgtagggcgctcgagatggagcccccagcttccttgatggatcgcggggcgcgagtgccctagacaagccggagctgggaccggcaatcgggcgttgatccttgtcacctgtcgcagaccctcatccctcccgtgggagccccctttggacactctatgaccctggaccctcgggggacctgaacttgatgcgatgggaggctgtgcaggctcgcggcggcgcttttcggattccgagggggaggagaccgtcccggagccccggctccctctgttggaccatcagggcgcgcattggaagaacgcggtgggcttctggctgctgggcctttgcaacaacttctcttatgtggtgatgctgagtgccgcccacgacatccttagccacaagaggacatcgggaaaccagagccatgtggacccaggcccaacgccgatcccccacaacagctcatcacgatttgactgcaactctgtctctacggctgctgtgctcctggcggacatcctccccacactcgtcatcaaattgttggctcctcttggccttcacctgctgccctacagcccccgggttctcgtcagtgggatttgtgctgctggaagcttcgtcctggttgccttttctcattctgtggggaccagcctgtgtggtgtggtcttcgctagcatctcatcaggccttggggaggtcaccttcctctccctcactgccttctaccccagggccgtgatctcctggtggtcctcagggactgggggagctgggctgctgggggccctgtcctacctgggcctcacccaggccggcctctcccctcagcagaccctgctgtccatgctgggtatccctgccctgctgctggccagctatttcttgttgctcacatctcctgaggcccaggaccctggaggggaagaagaagcagagagcgcagcccggcagcccctcataagaaccgaggccccggagtcgaagccaggctccagctccagcctctcccttcgggaaaggtggacagtgttcaagggtctgctgtggtacattgttcccttggtcgtagtttactttgccgagtatttcattaaccagggactttttgaactcctctttttctggaacacttccctgagtcacgctcagcaataccgctggtaccagatgctgtaccaggctggcgtctttgcctcccgctcttctctccgctgctgtcgcatccgtttcacctgggccctggccctgctgcagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggagaccagtgatgagcaccgggagtttgcaatggcggccacctgcatctctgacacactggggatctccctgtcggggctcctggctttgcctctgcatgacttcctctgccagctctcctgatactcgggatcctcaggacgcaggtcacattcacctgtgggcagagggacaggtcagacacccaggcccaccccagagaccctccatgaactgtgctcccagccttcccggcaggtctgggagtagggaagggctgaagccttgtttcccttgcaggggggccagccattgtctcccacttggggagtttcttcctggcatcatgccttctgaataaatgccgattttatccatggaaa

SEQ ID NO: 101 - DNA, 1797 bp, Homo sapiens
gtgctgtcacgtgatccgacaaacggcctctgcatagtgcagaacattctgctgctcttaaaggtacaggcctcagggtccctgctgtagacggggcgggggagagtacgatgggtggggcgtggtgggtcgtagggcgctcgagatggagcccccagcttccttgatggatcgcggggcgcgagtgccctagacaagccggagctgggaccggcaatcgggcgttgatccttgtcacctgtcgcagaccctcatccctcccgtgggagccccctttggacactctatgaccctggaccctcgggggacctgaacttgatgcgatgggaggctgtgcaggctcgcggcggcgcttttcggattccgagggctgctgggcctttgcaacaacttctcttatgtggtgatgctgagtgccgcccacgacatccttagccacaagaggacatcgggaaaccagagccatgtggacccaggcccaacgccgatcccccacaacagctcatcacgatttgactgcaactctgtctctacggctgctgtgctcctggcggacatcctccccacactcgtcatcaaattgttggctcctcttggccttcacctgctgccctacagcccccgggttctcgtcagtgggatttgtgctgctggaagcttcgtcctggttgccttttctcattctgtggggaccagcctgtgtggtgtggtcttcgctagcatctcatcaggccttggggaggtcaccttcctctccctcactgccttctaccccagggccgtgatctcctggtggtcctcagggactgggggagctgggctgctgggggccctgtcctacctgggcctcacccaggccggcctctcccctcagcagaccctgctgtccatgctgggtatccctgccctgctgctggccagctatttcttgttgctcacatctcctgaggcccaggaccctggaggggaagaagaagcagagagcgcagcccggcagcccctcataagaaccgaggccccggagtcgaagccaggctccagctccagcctctcccttcgggaaaggtggacagtgttcaagggtctgctgtggtacattgttcccttggtcgtagtttactttgccgagtatttcattaaccagggactttttgaactcctctttttctggaacacttccctgagtcacgctcagcaataccgctggtaccagatgctgtaccaggctggcgtctttgcctcccgctcttctctccgctgctgtcgcatccgtttcacctgggccctggccctgctgcagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggagaccagtgatgagcaccgggagtttgcaatggcggccacctgcatctctgacacactggggatctccctgtcggggctcctggctttgcctctgcatgacttcctctgccagctctcctgatactcgggatcctcaggacgcaggtcacattcacctgtgggcagagggacaggtcagacacccaggcccaccccagagaccctccatgaactgtgctcccagccttcccggcaggtctgggagtagggaagggctgaagccttgtttcccttgcaggggggccagccattgtctcccacttggggagtttcttcctggcatcatgccttctgaataaatgccgattttatccatgg

(19) United States
(12) Patent Application Publication
(10) Pub. No.: US 2022/0280545 A1
(43) Pub. Date: Sep. 8, 2022

(54) COMPOUNDS AND METHODS FOR MODULATING CLN3 EXPRESSION

(71) Applicants: Ionis Pharmaceuticals, Inc., Carlsbad, CA (US); Rosalind Franklin University of Medicine & Science, North Chicago, IL (US)

(72) Inventors: Michelle L. Hastings, North Chicago, IL (US); Frank Rigo, Carlsbad, CA (US)

(73) Assignees: Ionis Pharmaceuticals, Inc., Carlsbad, CA (US); Rosalind Franklin University of Medicine & Science, North Chicago, IL (US)

(21) Appl. No.: 17/274,981

(22) PCT Filed: Sep. 10, 2019
(86) PCT No.: PCT/US2019/050476

§ 371 Date: Mar. 10, 2021

Related U.S. Application Data
(60) Provisional application No. 62/891,127, filed on Aug. 23, 2019.
(60) Provisional application No. 62/729,067, filed on Sep. 10, 2018.

(51) Int. Cl. A61K 31/712; A61K 31/7125; A61K 31/713; A61K 47/02

(52) U.S. Cl. CPC A61K 31/712; A61K 31/7125; A61K 31/713; A61K 47/02

(57) ABSTRACT

Provided are compounds, methods, and pharmaceutical compositions for modulating the expression of CLN3 RNA in a cell or animal, and in certain instances modulating the expression of CLN3 protein in a cell or animal Such compounds, methods, and pharmaceutical compositions are useful to ameliorate at least one symptom or hallmark of a neurodegenerative disease. Such N symptoms and hallmarks include poor motor function, seizures, vision loss, poor cognitive function, psychiatric problems, accumulation of autofluorescent ceroid lipopigment, brain tissue dysfunction or cell death, accumulation of mitochondrial ATP synthase subunit C, accumulation of lipofuscin, or astrocyte activation in brain tissue.

### SEQUENCE LISTING

The present application is being filed along with a Sequence Listing in electronic format. The Sequence Listing is provided as a file entitled BIOL0343WOSEQ_ST25.txt, created on Sep. 10, 2019, which is 68 KB in size. The information in the electronic format of the sequence listing is incorporated herein by reference in its entirety.

### FIELD

Provided are compounds, methods, and pharmaceutical compositions for modulating the expression of CLN3 RNA in a cell or animal, and in certain instances modulating the expression of CLN3 protein in a cell or animal Such compounds, methods, and pharmaceutical compositions are useful to ameliorate at least one symptom or hallmark of a neurodegenerative disease. Such symptoms and hallmarks include poor motor function, seizures, vision loss, poor cognitive function, psychiatric problems, accumulation of autofluorescent ceroid lipopigment, brain tissue dysfunction or cell death, accumulation of mitochondrial ATP synthase subunit C, accumulation of lipofuscin, or astrocyte activation in brain tissue.

### BACKGROUND

Neuronal ceroid lipofuscinoses (NCL) is the general name for a family of neurodegenerative disorders that result from excessive accumulation of lipopigments, e.g. lipofuscin, in the body's tissues. Batten Disease, also known as Juvenile neuronal ceroid lipofuscinosis (JNCL), juvenile Batten Disease, cNCL, Spielmeyer-Vogt disease, or CLN3 Batten Disease, is the most common of the NCL disorders. Batten Disease occurs in approximately 1 in 25,000 births in the United States and Europe and has been reported in many other countries worldwide. Onset occurs between four and eight years of age and symptoms include progressive loss of motor function, seizures, vision loss, loss of cognitive function, and psychiatric problems, resulting in death before age 30. Batten Disease is an autosomal recessive disorder caused by mutations of the CLN3 (ceroid-lipofuscinosis, neuronal 3 gene). There are forty-nine known mutations of CLN3, but approximately 80% of Batten Disease cases result from a particular deletion of the CLN3 gene spanning exons 7 and 8 (CLN3Δ78). The CLN3Δ78 deletion causes a frame-shift that results in a premature stop codon in exon 9. This stop codon removes the lysosomal targeting sequence from the protein. The truncated protein product of CLN3Δ78 is 33% of the length of the wild type CLN3 protein, and is non-functional, or only partially functional. Furthermore, it is postulated that the shortened mRNA undergoes nonsense-mediated decay, leading to low levels of the shortened protein product.

Batten Disease is an autosomal recessive lysosomal storage disease. It is characterized by the accumulation of autofluorescent ceroid lipopigment in various organs, with only the brain tissue showing severe dysfunction and cell death. The accumulation of lipids and proteins are composed primarily of mitochondrial ATP synthase subunit C and lipofuscin, an insoluble pigment associated with aging. CLN3 localizes to lysosomal and endosomal membranes. The function of the CLN3 protein is not well understood, but it is implicated in many important processes, for example, membrane trafficking, phospholipid distribution, and response to oxidative stress. Currently, there are no treatments for any of the NCL disorders, and patient options are limited to remedial management of symptoms (see Bennett and Rakheja, Dev. Disabil. Res. Rev. 2013, 17, 254-259).

Currently there is a lack of acceptable options for treating neurodegenerative diseases such as juvenile Batten disease. It is therefore an object herein to provide compounds, methods, and pharmaceutical compositions for the treatment of such diseases.

### SUMMARY OF THE INVENTION

Provided herein are compounds, methods, and pharmaceutical compositions for modulating the expression of CLN3 RNA, and in certain embodiments modulating the expression of CLN3 protein in a cell or animal In certain embodiments, the animal has a neurodegenerative disease. In certain embodiments, the animal has juvenile Batten disease. In certain embodiments, compounds useful for modulating the expression of CLN3 RNA are oligomeric compounds. In certain embodiments, the oligomeric compound comprises a modified oligonucleotide. Provided herein are therapeutic splice-switching antisense oligonucleotides for juvenile Batten Disease. Provided herein are oligomeric compounds capable of inducing skipping of CLN3 exon 5.

Also provided are methods useful for ameliorating at least one symptom or hallmark of a neurodegenerative disease. In certain embodiments, the neurodegenerative disease is juvenile Batten disease. In certain embodiments symptoms and hallmarks include deficits in motor tasks, impaired motor skills, impaired motor coordination, intracellular accumulation of mitochondrial subunit C ATPase, GFAP activation, and astrocyte activation. In certain embodiments, amelioration of these symptoms results in improved motor tasks, improved motor skills, improved motor coordination, reduced ATPase subunit C accumulation, reduced GFAP activation, and reduced in astrocyte activation. In certain embodiments, provided herein are modified oligonucleotides for treating Batten Disease.

### BRIEF DESCRIPTION OF THE DRAWINGS

FIG. 1 and FIG. 1B: Deletion of exons 7 and 8 most common mutation. FIG. 1A provides a schematic of the CLN3 gene. The CLN3 gene includes 15 exons. A common mutation is deletion of exons 7 and 8 (Δ78) in 85% of patients. FIG. 1B provides a schematic of the CLN3 protein positioned in the lysosomal membrane. CLN3 protein is a lysosomal membrane protein having 438 amino acids that comprises six transmembrane segments. Both the amino terminal and the carboxy terminal segments of the CLN3 protein are predicted to be located in the cytoplasm of the lysosome; six putative transmembrane segments in order from the amino terminus to the carboxy terminus of 1, 2, 3, 4, 5, 6 are linked by amino acid sequences as follows: c-1-l-2-c-3-l-4-c-5-l-6-c, where/indicates an amino acid sequence located in the lumen and c indicates an amino acid sequence located in the cytoplasm.

FIG. 2: Deletion of CLN3 exons 7 and 8 results in a truncated protein. A schematic is provided of the CLN3Δex78 gene and the CLN3Δex78 protein. The CLN3Δex78 gene includes exons 1-6, 9, and 10-15. The omission of Exons 7 and 8 leads to a frame-shift mutation, resulting in a stop codon in Exon 9, which leads to either nonsense-mediated decay or a truncated protein, CLN3Δex78 protein. CLN3Δex78 protein has 181 amino acids, and comprises putative transmembrane segments 1, 2, and 3, and lacks a lysosomal targeting sequence. The amino terminal segment of the CLN3Δex78 protein is predicted to be located outside of the lysosome, while the carboxy terminal segment is predicted to be located in the lumen. The C-terminal truncation of the CLN3Δex78 protein results in a loss of the lysosomal targeting sequence located in the C-terminus of the CLN3 protein. Three transmembrane segments in order from the amino terminus to the carboxy terminus are linked by amino acid sequences as follows: c-1-l-2-c-3-l where l indicates an amino acid sequence located in the lumen and c indicates an amino acid sequence located in the cytoplasm.

FIGS. 3A-3E: Splice switching oligonucleotides (SSOs) to modify splicing, providing an overview of modified oligonucleotides (splice switching oligonucleotides (SSOs)), to modify splicing. FIG. 3A provides certain examples of characteristics of modified oligonucleotides (splice switching oligonucleotides): alter pre-mRNA splicing; modified nucleic acids; short oligomers; stable, RNase H resistant; safe, low toxicity; freely taken up by many cells; FDA approved for treatment of other pediatric diseases. FIG. 3B provides a structure depicting an example of a portion of a modified oligonucleotide (splice switching oligonucleotide), comprising a first modified nucleobase comprising a modified sugar 2′O-methoxyethyl (2′-MOE), linked by a modified internucleoside linkage, phosphorothioate (PS), to a second nucleobase. FIG. 3C provides a schematic of a gene, transcription of the gene to mRNA, and the binding of a modified oligonucleotide (SSO) to the mRNA. FIG. 3D provides an example of an FDA news release of an FDA approved modified oligonucleotide (S SO) drug, SPINRAZA® (nusinersen) injection 12 mg/5 mL, FDA approves first drug for spinal muscular atrophy; new therapy addresses unmet medical need for rare disease. FIG. 3E provides an example of an FDA news release of an FDA approved modified oligonucleotide (SSO) drug, EXONDYS 51® (eteplirsen) injection, FDA grants accelerated approval to first drug for Duchenne muscular dystrophy.

FIGS. 4A-4C: SSOs to correct the CLN3Δex78 reading frame. FIG. 4A is a schematic of CLN3Δex78 pre-mRNA including Exons 1-6, and Exons 9-15. Exons 1-6 and 9-15 are depicted as boxes, introns between each exon are depicted as lines, and splicing is depicted as diagonal lines between exons. An example of a modified oligonucleotide (SSO) is depicted as a comb-like figure, including an example of a part of the SSO comprising a nucleobase sequence GCAGC . . . binding to a part of exon 5. Binding of the modified oligonucleotide (SSO) leads to exon 5 skipping, as depicted by splicing (diagonal lines) of Exon 4 to Exon 6. FIG. 4B provides a schematic of CLN3Δex578 mRNA including Exons 1-4, 6, and 9-15. FIG. 4C is a schematic of CLN3Δex578 protein, a 340 amino acid protein; 4 transmembrane segments are predicted to be positioned in the lysosomal membrane. Both the amino terminal and the carboxy terminal segments of the CLN3Δex578 protein are predicted to be located in the cytoplasm of the lysosome based on modelling; four transmembrane segments in order from the amino terminus to the carboxy terminus of 1, 3, 5, 6 are linked by amino acid sequences as follows: c-1-l-3-c-5-l-6-c, where l indicates an amino acid sequence located in the lumen and c indicates an amino acid sequence located in the cytoplasm.

FIGS. 5A-5E: SSO induced skipping of CLN3 exon 5, an SSO candidate screen for exon 5 skipping. FIGS. 5A-5C provide alignments of and data obtained from modified oligonucleotide (splice-switching oligonucleotide, SSO) induced skipping of mouse CLN3 exon 5, for modified oligonucleotides (SSOs) 1-33 (corresponding to SEQ ID NOs: 3-35). FIG. 5A is a schematic of a map of SSOs 1-33, each comprising a complementary sequence to the mouse CLN3 pre-mRNA sequence in the mCLN3 exon 5 region, and surrounding pre-mRNA introns. Modified oligonucleotide (SSO) locations are represented as numbered lines 1 to 33 on mouse CLN3 (mCLN3) pre-mRNA. Intron 4 and intron 5 are represented by black lines and exon 5 (mCLN3 exon 5) is represented by a gray box; the gray box indicates exon 5 and lines indicate the flanking introns. The depicted target region of intron 4 is nucleotides 4,807 to 4,866 of SEQ ID NO: 2, exon 5 is nucleotides 4,867 to 4,946 of SEQ ID NO:2, and the depicted target region of intron 5 is nucleotides 4,947 to 4,984 of SEQ ID NO:2. FIG. 5B provides results of an in vitro candidate screen of modified oligonucleotides (SSOs) 1-33. Real-time PCR (RT-PCR) was performed on RNA extracted from mouse CLN3 Δ78/6,78 cells individually transfected with the indicated modified oligonucleotide (SSO) at the top of each lane, and products were separated on an acrylamide gel. The percent of exon 5 skipped is indicated below the gel. “M” indicates mock treated and “UT” untreated. The top band (Δex78) represents a shortened, disease-associated CLN3Δex78 RNA that contains a premature stop codon in exon 9. The lower band (Δex578) represents the CLN3Δex578 RNA that lacks exons 5, 7, and 8 and has exon 6 and a restored reading frame for exons 9-15. The numerical quantification of percent of the transcripts in which exons 5, 7, and 8 are skipped, (Δex578 (%)) is determined as [Δ578/(Δ578+Δ78)]×100], is displayed below the gel, and is presented in Example 1, Table 1. FIG. 5C provides a sequence alignment of mouse SSO-26 (SSO 26, SSO # 26; Compound ID 730500; SEQ ID NO: 28) with the mouse CLN3 pre-mRNA sequence (nucleotides 4,927 to 4,961 of SEQ ID NO: 2). In the pre-mRNA sequence, exon 5 is depicted in capital letters, intron 5 is depicted in lowercase letters, and an arrow marks the 5′ splice site. FIG. 5D provides the results of an in vivo analysis of certain modified oligonucleotides of FIG. 5A: Cln3 spliced products amplified from hippocampal cDNA made from RNA were isolated from adult homozygous Cln3Δex7/8 mice two weeks post-ICV treatment with PBS (−) or 500 μg of the indicated modified oligonucleotide (ASO). FIG. 5E provides a graph of the quantification of the percent of RT-PCR Cln3Δex5/7/8 product [Δ578/(Δ578+Δ78)]×100 of the analysis of FIG. 5D. Error bars represent s.e.m. One-way ANOVA with Dunnett's multiple comparisons test to control-treated (−) samples. F(7,13)=31.36. **P<0.01, ****P<0.0001, N=2-3 mice as on gel. Mouse 13 was a Cln3+/Δex7/8.

FIG. 6: CLN3Δ78 knock-in mice, an overview is provided of the CLN3Δ78 knock-in mouse model, discussed in Example 5, indicating that these mice have deficits in motor tasks by 8-12 weeks, intracellular accumulation of autofluorescent storage material made up of mitochrondrial subunit C ATPase, astrocyte activation. The mouse model is discussed in, for example, Cotman et al., (2002), Hum. Mol. Genet., 11:2709.

FIGS. 7A-7C: Delivery analysis: SSOs distribute throughout the CNS, providing the results of an assay of the distribution of modified oligonucleotide mouse SSO-26 in neonatal mice, as discussed in Example 4. An analysis of the delivery of the modified oligonucleotides determined that modified oligonucleotides (SSOs) distribute throughout the CNS. Intracerebroventricular (ICV) injection of modified oligonucleotide SSO-26 shows widespread delivery in the brain. SSO-26 was administered via neonatal ICV injection in Cln3 Δ78/Δ78 mice and 3 weeks post injection, modified oligonucleotide (SSO) delivery was analyzed. Distribution of modified oligonucleotide was analyzed by immunofluorescence of modified oligonucleotide (SSO) and Hoechst staining of nuclei marker. FIG. 7A provides a schematic of the treatment of neonatal CLN3Δ78/Δ78 mice by intracerebroventricular (ICV) injection of modified oligonucleotide SSO-26 on post-natal day one (P1); three weeks post-injection, delivery analysis was performed. FIG. 7B provides the results of delivery analysis in, from left to right, hippocampus, somatosensory cortex (ss cortex), and thalamus. Four images are provided for each tissue, at 10×magnification. Immunoflourescent staining to detect modified oligonucleotide is shown in the left column of each set of images, and Hoechst staining to detect modified oligonucleotide is shown in the right column of each set of images. The top row for each tissue provides images obtained from CLN3Δ78/Δ78 mice treated with modified oligonucleotide SSO-26 (Δ78/Δ78 SSO-26) and the bottom row for each tissue provides images obtained from CLN3Δ78/Δ78 mice not treated with an SSO (Δ78/Δ78 Untreated). FIG. 7C provides the results at 60×magnification. The treated animals display modified oligonucleotide staining in the hippocampus, somatosensory cortex, and thalamus, while no signal is detected in the modified oligonucleotide panels for untreated animals Similar levels of staining are seen for both treated and untreated animal tissues using Hoechst staining, indicating that the tissues imaged contain approximately the same number of cells.

FIG. 8: Testing mouse modified oligonucleotide SSO-26 in vivo, providing a schematic of a testing modified oligonucleotide SSO-26 in vivo, providing a timeline for the experiment discussed in Example 7. Either naked modified oligonucleotide SSO-26 or a naked control oligonucleotide (SEQ ID NO: 97) was administered to mice by ICV injection on post-natal day one (P1, Treatment). Behavioral analysis was conducted at 8 weeks of age (Rotarod and Pole test); analysis was conducted at 19 weeks of age (Splicing and Histology).

FIGS. 9A-9C: SSOs induce exon skipping in vivo for up to 19 weeks, providing results of the experiment provided in Example 7, showing that modified oligonucleotides (SSOs) induce exon skipping in vivo for up to 19 weeks. FIG. 9A provides a schematic of a timeline; either mouse modified oligonucleotide SSO-26 or control modified oligonucleotide SSO-C (control SEQ ID NO: 97) was administered to mice by ICV injection on post-natal day one (P1, Treatment). Exon skipping analysis (splicing analysis) was conducted at 19 weeks of age. FIG. 9B provides the result of RT-PCR analysis of RNA extracted from the hippocampus of the treated CLN3Δ78/Δ78 mice (Genotype: Cln3 Δ78/Δ78). The left four lanes provide the results from individual SSO-C treated mice, and the right four lanes provide the results from individual SSO-26 treated mice. The top band (Δex78) represents a shortened, disease-associated CLN3Δex78 RNA that contains a premature stop codon in exon 9. The lower band (Δex578) represents a CLN3Δex578 RNA that lacks exons 5, 7, and 8 and has exon 6 and a restored reading frame for exons 9-15. The top band is present in both the SSO-C and the SSO-26 treated mice, while the lower band is seen only in the SSO-26 treated mice. FIG. 9C provides a graph of the percentage of transcripts representing mRNA without exon 5 (Exon 5 Skipped (%)) [Δ578/(Δ578+Δ78)]×100] in CLN3Δ78/Δ78 mice treated with SSO-C (Δ78/Δ78 SSO-C) or in CLN3Δ78/Δ78 mice treated with SSO-26 (Δ78/Δ78 SSO-26).

FIGS. 10A-10C: SSO-26 reduces ATPase subunit C accumulation, providing results of the experiment discussed in Example 7, showing that modified oligonucleotide SSO-26 reduces ATPase subunit C accumulation in the hippocampus. FIG. 10A provides a schematic of a timeline; either SSO-26 or SSO-C was administered to CLN3Δ78/Δ78 mice by ICV injection on post-natal day one (P1, Treatment). As an additional control, heterozygous CLN3+/Δ78 mice were injected with the control oligonucleotide on post-natal day one. Mice were sacrificed at 19 weeks, and analyzed for ATPase subunit C accumulation (Analysis). FIG. 10B provides images of staining of histological sections of the hippocampus. From left to right, images are provided of sections obtained from heterozygous CLN3+/Δ78 mice injected with the control oligonucleotide (+/Δ78 SSO-C), CLN3Δ78/Δ78 mice injected with the control modified oligonucleotide (Δ78/Δ78 SSO-C), and CLN3Δ78/Δ78 mice injected with SSO-26 (Δ78/Δ78 SSO-26). The top row provides images stained for ATP synthase subunit C (subunit C), and the bottom row provides images stained for ATP synthase subunit C overlaid with Hoechst nuclear stain (subunit C Hoechst). FIG. 10C provides a graph of the percent area of the total image that stains positive for ATPase subunit C (Subunit C % area) for each of the three columns of images of FIG. 10B). The data in FIG. 10C is presented in Example 7, Table 7.

FIGS. 11A-11C: SSO-26 reduces ATPase subunit C accumulation, providing results of the experiment discussed in Example 7, showing that modified oligonucleotide SSO-26 reduces ATPase subunit C accumulation in the thalamus. FIG. 11A provides a schematic of a timeline; either SSO-26 or SSO-C was administered to CLN3Δ78/Δ78 mice by ICV injection on post-natal day one (P1, Treatment). As an additional control, heterozygous CLN3+/Δ78 mice were injected with the control modified oligonucleotide on post-natal day one. Mice were sacrificed at 19 weeks, and analyzed for ATPase subunit C accumulation (Analysis). FIG. 11B provides images of staining of histological sections of the thalamus. From left to right, images are provided of sections obtained from heterozygous CLN3+/Δ78 mice injected with the control oligonucleotide (+/Δ78 SSO-C), CLN3Δ78/Δ78 mice injected with the control oligonucleotide (Δ78/Δ78 SSO-C), and CLN3Δ78/Δ78 mice injected with SSO-26 (Δ78/Δ78 SSO-26). The top row provides images stained for ATP synthase subunit C (subunit C), and the bottom row provides images stained for ATP synthase subunit C overlaid with Hoechst nuclear stain (subunit C Hoechst). FIG. 11C provides a graph of the percent area of the total image that stains positive for ATPase subunit C (Subunit C (% area)) for each of the three columns of images of FIG. 11B). The data in FIG. 11C is presented in Example 7, Table 7.

FIGS. 12A-12B: SSO-26 attenuates astrocyte activation, providing results of the experiment discussed in Example 7, showing that modified oligonucleotide SSO-26 attenuates astrocyte activation. Modified oligonucleotide SSO-26 reduces astrocyte activation in Cln3 Δ78/Δ78 mice. Mice were treated as discussed in FIG. 10, and sacrificed at 19 weeks. FIG. 12A: Analysis of glial fibrillary acidic protein (GFAP) in the somatosensory (ss) and visual cortex, and thalamus of 19 week old Cln3+/Δ78 and Cln3Δ78/Δ78 mice treated as neonates with either control modified oligonucleotide SSO (SSO-C) or modified oligonucleotide SSO-26; provided are images of histological sections of the somatosensory cortex (ss cortex, top row), visual cortex (middle row), and thalamus (bottom row) from the treated mice, stained for GFAP. From left to right, images are provided of sections obtained from heterozygous CLN3+/Δ78 mice injected with the control oligonucleotide (+/Δ78 SSO-C), CLN3Δ78/Δ78 mice injected with the control oligonucleotide (Δ78/Δ78 SSO-C), and CLN3Δ78/Δ78 mice injected with SSO-26 (478/478 SSO-26). FIG. 12B Quantitative analysis of GFAP accumulation in the corresponding regions, displayed as mean ±s.e.m, provided is a graph of the percent area of the total image that stains positive for GFAP (GFAP (% area)) for each of the three images of stained histological sections of the somatosensory cortex of FIG. 12A. Statistical significance was determined by one way ANOVA with Dunne8's multiple comparisons test. *p<0.05, ***p<0.001, ****p<0.0001. FIG. 12C: Quantitative analysis of GFAP accumulation in the corresponding regions, displayed as mean±s.e.m; provided is a graph of the percent area of the total image that stains positive for GFAP (GFAP (% area)) for each of the three images of stained histological sections of the visual cortex of FIG. 12A. FIG. 12D provides a graph of the percent area of the total image that stains positive for GFAP (GFAP (% area)) for each of the three images of stained histological sections of the thalamus of FIG. 12A. Statistical significance was determined by one way ANOVA with Dunne8's multiple comparisons test. *p<0.05, ***p<0.001, ****p<0.0001.

FIGS. 13A-13C: SSO-26 improves motor skills (rotarod); modified oligonucleotide SSO-26 treatment rescues motor deficits in Cln3Δ78/Δ78 mice; provided are results of the experiment discussed in Example 7, showing that modified oligonucleotide SSO-26 improves motor behavior; modified oligonucleotide SSO-26 improves motor skills (rotarod). FIG. 13A Cln3+/Δ78 and Cln3Δ78/Δ78 mice treated with SSO-C or SSO-26 at P1/2 (post-natal day 1 or 2), were assessed for motor function on an accelerating rotarod at 8 weeks of age; provided is a schematic of a timeline; either SSO-26 or SSO-C (control SEQ ID NO: 97) was administered to CLN3Δ78/Δ78 mice by ICV injection on post-natal day one (P1, Treatment). As an additional control, heterozygous CLN3+/Δ78 mice were injected with the control modified oligonucleotide on post-natal day one. Rotarod analysis, by accelerating rotarod, was conducted at 8 weeks of age (Behavior). FIG. 13B is a photo of the rotarod apparatus. FIG. 13C is a graph of the latency to fall (Latency to fall(s) for, from left to right, heterozygous CLN3+/Δ78 mice treated with the control oligonucleotide (+/Δ78 SSO-C), CLN3Δ78/Δ78 mice treated with the control oligonucleotide (Δ78/Δ78 SSO-C) or CLN3Δ78/Δ78 mouse treated with SSO-26 (Δ78/Δ78 S SO-26). The latency to fall on the accelerating rotarod plotted as mean±s.e.m. **p<0.01, ***p<0.001, ****p<0.0001. The data in FIG. 13C are presented in Example 7, Table 6.

FIGS. 14A-14C: SSO-26 treatment improves pole test performance; modified oligonucleotide SSO-26 treatment rescues motor deficits in Cln3Δ78/Δ78 mice; provided are results of the experiment discussed in Example 7, showing that modified oligonucleotide SSO-26 treatment improves motor behaviors, and modified oligonucleotide SSO-26 treatment improves pole test performance. FIG. 14A: Cln3+/Δ78 and Cln3 Δ78/Δ78 mice treated with SSO-C or SSO-26 at P1/2, were assessed for motor function on a vertical pole test at 8 weeks of age; provided is a schematic of a timeline; either SSO-26 or SSO-C was administered to CLN3Δ78/Δ78 mice by ICV injection on post-natal day one (P1, Treatment). As an additional control, heterozygous CLN3+/Δ78 mice were injected with the control oligonucleotide on post-natal day one. Pole test performance, vertical pole test: turn around, was conducted at 8 weeks of age (Behavior). FIG. 14B is a photo of the pole test. FIG. 14C is a graph of the time to turn (Time to turn(s)) for, from left to right, heterozygous CLN3+/Δ78 mice treated with the control oligonucleotide (+/Δ78 SSO-C), CLN3Δ78/Δ78 mice treated with a control modified oligonucleotide (Δ78/Δ78 SSO-C) or CLN3Δ78/Δ78 mouse treated with SSO-26 (Δ78/Δ78 SSO-26). The average time to turn downward, 180° on a vertical pole is plotted as mean±s.e.m. Statistical significance was determined using one way ANOVA and Tukey's multiple comparisons test. **p<0.01, ***p<0.001, ****p<0.0001. The data in FIG. 14C are presented in Example 7, Table 6.

FIGS. 15A-15B: SSO-26 induces stable exon 5 splicing for up to 26 weeks, providing results of the experiment discussed in Example 7, showing that modified oligonucleotide S SO-26 induces stable exon 5 splicing for up to 26 weeks. Mice were treated as discussed in FIG. 10, with either modified oligonucleotide SSO-26 or modified oligonucleotide SSO-C; as an additional control, heterozygous CLN3+/Δ78 mice were injected with the control modified oligonucleotide on post-natal day one. Exon 5 skipping analysis was conducted at 26 weeks of age. FIG. 15A provides the result of RT-PCR analysis of RNA extracted from the hippocampus of, from left to right, CLN3+/Δ78 mice injected with the control oligonucleotide (lanes 1-4, Cln3+/Δ78), CLN3Δ78/Δ78 mice injected with the control oligonucleotide (lanes 5-8, Δ78/Δ78 SSO-C), and CLN3Δ78/Δ78 mice injected with SSO-26 (lanes 9-11, Cln3 Δ78/Δ78 SSO-26). The top band, labeled FL represents the full-length, wild-type CLN3 transcript, the band immediately below labeled, Δex78, represents the disease-associated CLN3Δ78 transcript, and the bottom band, labeled Δex578, represents the modified disease-associated CLN3Δ78 RNA with exon 5 spliced out. FIG. 15B provides a graph of the percentage of transcripts representing mRNA without exon 5 (Exon 5 Skipped (%)) in from left to right, heterozygous CLN3+/Δ78 mice treated with the control oligonucleotide (+/Δ78 SSO-C), CLN3Δ78/Δ78 mice treated with a control oligonucleotide (Δ78/Δ78 SSO-C) or CLN3Δ78/Δ78 mouse treated with SSO-26 (Δ78/Δ78 SSO-26). This data is presented in Example 5, Table 4.

FIGS. 16A-16C: hCLN SSO walk in CLN3 WT/Δ78 fibroblast; Modified oligonucleotides (SSOs) induce skipping of CLN3 exon 5 in vitro; provided are results of the experiment discussed in Example 9. Example 9 provides examples of modified oligonucleotides that modulate the expression of human CLN3 RNA in vitro by inducing skipping of human CLN3 exon 5 in vitro. The Figures provide the results of an analysis of an hCLN3 modified oligonucleotide walk in CLN3 WT/Δ78 fibroblast (CLN3+/Δ78). FIG. 16A: Identification of the modified oligonucleotides (SSOs) that induce the most exon 5 skipping in human and CLN3. The gray box indicates exon 5 and lines the flanking introns. Modified oligonucleotide (SSO) locations are represented as numbered lines 1 to 40 on hCLN3 exon 5 pre-mRNA; provided is a schematic of human modified oligonucleotides (SSOs) #1-40 (corresponding to SEQ ID Nos: 57-90), each comprising a complementary sequence to the human CLN3 pre-mRNA sequence, in the hCLN3 exon 5 region, and surrounding pre-mRNA introns. Intron 4 and intron 5 are depicted by lowercase letters and exon 5 is depicted in uppercase letters surrounded by a gray box (the depicted target region has a sequence of cgtggttgggagggttgtcccctggaagctctgcggtctcactctattctcctgtcccagGCTGTGCTCCTGGCGGACATCCTCCCCACACTCGT CATCAAATTGTTGGCTCCTCTTGGCCTTCACCTGCTGCCCTACAGgtctgggtgagggtagtgggaggcagggtgggcaggagctg agaaaggggaggctgggatggc (SEQ ID NO: 98); intron 4 includes nucleotides 5,449 to 5,558 of SEQ ID NO:1; exon 5 includes nucleotides 5,559 to 5,638 of SEQ ID NO:1; and intron 5 includes nucleosides 5,639 to 5,701 of SEQ ID NO:1). The 3′ splice site (3′ ss) and the 5′ splice site (5′ ss) are indicated by arrows. RT-PCR was performed on RNA extracted from human CLN3+/Δ78 fibroblasts individually transfected with the indicated modified oligonucleotide (SSO), and products were separated on an acrylamide gel. FIG. 16B provides two images of an acrylamide gel showing exon 5 skipping in CLN3+/Δ78 fibroblasts. RT-PCR was performed on RNA extracted from human CLN3+/Δ78 cells individually transfected with the indicated modified oligonucleotide (S SO) and products were separated on an acrylamide gel. The percent of exon 5 skipped is indicated below the gel. “M” indicates mock treated and “UT” untreated. These fibroblasts express both full-length, wild-type CLN3 RNA (FL) and the shortened, disease-associated CLN3Δex78 transcript. The top band, labeled FL, represents the full-length, wild-type CLN3 transcript, the band immediately below the FL band, labeled Δex5, represents a modified FL RNA with exon 5 spliced out. The next band, labeled Δex78, represents the disease-associated CLN3Δ78/Δ78 RNA, and the next band, labeled Δex578 represents the modified disease-associated CLN3Δ78/Δ78 RNA with exon 5 spliced out. Each lane is numbered at the top to correspond to modified oligonucleotide (SSO) number. The numerical quantification of percent exon 5 skipped is calculated by [Δ578/(Δ578+Δ78)]×100], dividing Δex758 CLN3 transcripts, by total Δex578+Δ78 CLN3 transcripts and multiplying by 100. The percent exon 5 skipped is displayed below the gel, and is presented in Example 9, Table 10.

FIG. 17 provides an overview of conclusions related to the experiments portrayed in FIGS. 1-16, and discussed herein. Modified oligonucleotides (SSOs) induce skipping of CLN3 exon 5 to correct the CLN3 Δ78 reading frame in CLN3Δ78/Δ78 mice. Modified oligonucleotides (SSOs) are distributed widely throughout the CNS following a single neonatal ICV injection (of mice). Modified oligonucleotide SSO-26 reduces ATPase subunit C accumulation and GFAP activation. Modified oligonucleotide (SSO) treatment improves motor coordination in CLN3Δ78/Δ78 mice.

FIG. 18 provides an overview of symptoms, hallmarks, and causes of CLN3 Batten disease. Onset: 4-10 years old. Symptoms: vision loss, seizures, slow learning, speech difficulties, and loss of motor coordination Cellular hallmarks: accelerated accumulation of auto fluorescent material in the brain. Cause: mutations in CLN3. Predominant mutation: deletion of exon 7 and 8 resulting in a reading frame-shift and premature termination codon.

FIG. 19 provides an overview of modified oligonucleotides (splice-switching antisense oligonucleotides (SSO)) as follows: modified nucleic acids; 15-25 nucleotides long; stable and RNase H resistant; low-toxicity; freely taken up by many cells in vivo; bind via complementary base pairing to target mRNA to alter pre-mRNA splicing.

FIGS. 20A and 20B provide an overview of a therapeutic approach. FIG. 20A provides an overview to the approach. Modified oligonucleotides (SSOs) can promote CLN3 exon 5 skipping to restore the mRNA reading frame. Reading frame correction will partially restore CLN3 function. FIG. 20B provides a schematic of the approach, depicting, from left to right, pre-mRNA, mRNA, and proposed protein models. The figure depicts CLN3, CLN3Δex78, and the modified oligonucleotide (SSO)-induced CLN3Δ578 isoforms. Exons are depicted as boxes, introns as lines, and splicing as the diagonal lines. Exon 5 skipping results in a frame-shifted exon 6, which is corrected in exon 9. Exon 5 skipping in CLN3Δ78 cells results in a CLN3Δ578 mRNA, which is shorter than the wild type CLN3 mRNA, and shorter than CLN3Δ78 mRNA, but no longer includes the premature stop codon of CLN3Δ78 that occurs because of frame-shifting. A proposed model of the protein is shown as well as the predicted membrane protein resulting from the modified oligonucleotide (SSO)-mediated exon skipping. The frame-shifting is corrected by skipping exon 5. This correction results in a shorter protein, including only transmembrane segments 1, 3, 5, and 6, compared to the wild type full length protein including transmembrane segments 1-6, but is longer than the dysfunctional CLN3Δ78 protein, which, because of the premature stop codon, only include transmembrane segments 1, 2, and 3.

FIGS. 21A-21I: SSO-26 induces stable exon 5 splicing for up to 26 weeks, providing the results of modulation of CLN3 RNA expression assays of modified oligonucleotide SSO-26. RT-PCR analysis of RNA extracted from the hippocampus of Cln3+/Δ78 and Cln3 Δ78/Δ78 mice at 3 (FIGS. 21A-21C), 19 (FIGS. 21D-21F), and 26 (FIGS. 21G-21I) weeks post-treatment at P1 with control modified oligonucleotide SSO-C or modified oligonucleotide SSO-26. These Figures provide results of the experiment discussed in Example 7, showing that SSO-26 induces exon 5 splicing. FIG. 21A: SSO-26 was administered to mice by ICV injection on post-natal day one (P1, Treatment), and splicing analysis was conducted at 3 weeks of age. FIG. 21B: Exon skipping analysis (splicing analysis) was conducted at three weeks of age. RT-PCR analysis of RNA extracted from the hippocampus of treated CLN3Δ78/Δ78 mice. The left five lanes provide the results obtained from modified oligonucleotide (SSO)-treated CLN3+/Δ78 (Cln3+/Δ78) mice, and the right seven lanes provide the results from individual SSO-26 treated CLN3+/Δ78 mice. The top band, labeled FL, represents the full-length, wild-type CLN3 transcript, the band immediately below the FL band, labeled 4ex5, represents a modified FL RNA with exon 5 spliced out. The next band, labeled Δex78, represents the disease-associated CLN3Δ78/Δ78 transcript, and the next band, labeled Δex578 represents the modified disease-associated CLN3Δ78/Δ78 RNA with exon 5 spliced out. The lower band (Δex578) represents the CLN3Δex578 RNA that lacks exons 5, 7, and 8 and has exon 6 and a restored reading frame for exons 9-15. The top band, and the Δex5 band are present in the modified oligonucleotide SSO-26 treated CLN3+/Δ78 mice only. FIG. 21C: the right panel provides a graph of the percentage of transcripts representing mRNA without exon 5 (Exon 5 Skipping (%)) in CLN3Δ78/Δ78 mice (Δ78/Δ78 SSO-26) and in CLN3+/Δ78 mice treated with SSO-26 (+/Δ78 SSO-26), calculated as [Δ578/(Δ578+Δ78)]×100]. FIGS. 21D-21F provide results of the experiment provided in Example 7, showing that modified oligonucleotides (SSOs) induce exon skipping in vivo for up to 19 weeks. FIG. 21D provides a schematic of a timeline; either SSO-26 or SSO-C was administered to mice by ICV injection on post-natal day one (P1, Treatment). Exon skipping analysis (splicing analysis) was conducted at 19 weeks of age. FIG. 21E provides the result of RT-PCR analysis of RNA extracted from the hippocampus of the treated mice. The mouse genotype is indicated above the gel. The left eight lanes provide the results from individual SSO-C treated mice, and the right four lanes provide the results from individual SSO-26 treated mice. The left four lanes provide the results from CLN3+/Δ78 mice (Cln3+/Δ78) and the right eight lanes provide the results from CLN3Δ78/Δ78 mice (Cln3Δ78/Δ78). The top band, labeled FL, represents a full length wild type CLN3 transcript. The middle band, labeled Δex78, represents a shortened, disease-associated CLN3Δex78 RNA that contains a premature stop codon in exon 9. The lower band, labeled Δex578, represents a CLN3Δex578 RNA that lacks exons 5, 7, and 8 and has exon 6 and a restored reading frame for exons 9-15. The FL band is present in the SSO-C treated CLN3+/Δ78 mice; the lower Δ578 band is seen only in the SSO-26 treated CLN3Δ78/Δ78 mice. FIG. 21F provides a graph of the percentage of transcripts representing mRNA without exon 5 (Exon 5 Skipped (%)) in CLN3Δ78/Δ78 mice treated with SSO-C (Δ78/Δ78 SSO-C) or in CLN3Δ78/Δ78 treated with SSO-26 (Δ78/Δ78 SSO-26), calculated as [Δ578/(Δ578+Δ78)]×100]. FIGS. 21G-21I provide results of the experiment discussed in Example 7, showing that SSO-26 induces stable exon 5 splicing for up to 26 weeks. FIG. 21G provides a schematic of a timeline; either SSO-26 (SSO-26, or SSO-C was administered to mice by ICV injection on post-natal day one (P1, Treatment). Exon skipping analysis (splicing analysis) was conducted at 26 weeks of age. FIG. 21H provides the result of RT-PCR analysis of RNA extracted from the hippocampus of the treated mice. The mouse genotype is indicated above the gel. The left eight lanes provide the results from individual SSO-C treated mice, and the right four lanes provide the results from individual SSO-26 treated mice. The left four lanes provide the results from CLN3+/Δ78 mice (Cln3+/Δ78) and the right eight lanes provide the results from CLN3Δ78/Δ78 mice (Cln3Δ78/Δ78). The top band, labeled FL, represents a full length wild type CLN3 transcript. The middle band, labeled Δex78, represents a shortened, disease-associated CLN3Δex78 RNA that contains a premature stop codon in exon 9. The lower band, labeled Δex578, represents a CLN3Δex578 RNA that lacks exons 5, 7, and 8 and has exon 6 and a restored reading frame for exons 9-15. The FL band is present in the SSO-C treated CLN3+/Δ78 mice; the lower Δ578 band is seen only in the SSO-26 treated CLN3Δ78/Δ78 mice. FIG. 21I provides a graph of the percentage of transcripts representing mRNA without exon 5 (Exon 5 Skipped (%)), calculated as [Δ578/(Δ578+Δ78)]×100], in CLN3Δ78/Δ78 mice treated with SSO-C (Δ78/Δ78 SSO-C) or in CLN3Δ78/Δ78 treated with SSO-26 (Δ78/Δ78 SSO-26). Data show mean±s.e.m. ****p<0.0001 (one way ANOVA; Dunne8's multiple comparisons test). This data is presented in Example 5, Table 4.

FIGS. 22A-22E: Modified oligonucleotide SSO-26 treatment reduces ATPase subunit C accumulation in the brain of mutant mice; Immunofluorescent staining for mitochondrial ATP synthase subunit C, the nuclei were stained with Hoechst, in the hippocampus (FIGS. 22B and 22C) and thalamus (FIGS. 22D and 22E) of 19 week old Cln3+/Δ78 and Cln3 Δ78/Δ78 mice treated with either control modified oligonucleotide SSO-C or modified oligonucleotide SSO-26 at post-natal day 1 or 2 (P1 or 2). Provided are results of the experiment discussed in Example 7, showing that SSO-26 reduces ATPase subunit C. FIG. 22A provides a schematic of a timeline; either SSO-26 or SSO-C was administered to CLN3Δ78/Δ78 mice by ICV injection on post-natal day one (P1, Treatment). As an additional control, heterozygous CLN3+/Δ78 mice were injected with the control oligonucleotide on post-natal day one. Mice were sacrificed at 19 weeks, and analyzed for ATPase subunit C accumulation (Analysis). FIG. 22B: Quantitative analysis of ATPase subunit C accumulation in the hippocampus; provided are images of staining of histological sections of the hippocampus. From left to right, images are provided of sections obtained from heterozygous CLN3+/Δ78 mice injected with the control oligonucleotide (+/Δ78 SSO-C), CLN3Δ78/Δ78 mice injected with the control oligonucleotide (Δ78/Δ78 SSO-C), and CLN3Δ78/Δ78 mice injected with SSO-26 (Δ78/Δ78 SSO-26). The top row provides images stained for ATP synthase subunit C (subunit C), and the bottom row provides images stained for ATP synthase subunit C overlaid with Hoechst nuclear stain (subunit C+Hoechst). FIG. 22C provides a graph of the percent area of the total image that stains positive for ATPase subunit C (Subunit C (% area)) for each of the three columns of images of FIG. 22B). The data in FIG. 22C is presented in Example 7, Table 7. FIG. 22D: Quantitative analysis of ATPase subunit C accumulation in the thalamus; provided are images of staining of histological sections of the thalamus. From left to right, images are provided of sections obtained from heterozygous CLN3+/Δ78 mice injected with the control oligonucleotide (+/Δ78 SSO-C), CLN3Δ78/Δ78 mice injected with the control oligonucleotide (Δ78/Δ78 SSO-C), and CLN3Δ78/Δ78 mice injected with SSO-26 (Δ78/Δ78 SSO-26). The top row provides images stained for ATP synthase subunit C (subunit C), and the bottom row provides images stained for ATP synthase subunit C overlaid with Hoechst nuclear stain (subunit C+Hoechst). FIG. 22E provides a graph of the percent area of the total image that stains positive for ATPase subunit C (Subunit C % area) for each of the three columns of images of FIG. 22D). Columns and bars represent mean±s.e.m. Statistical significance was determined by one way ANOVA with Dunne8's multiple comparisons test. *p<0.05, **p<0.01, ***p<0.001, ****p<0.0001. The data in FIGS. 22D and 22E are presented in Example 7, Table 7.

FIG. 23 provides an overview of conclusions drawn from the data presented in FIGS. 1-22, and discussed herein. Modified oligonucleotides (SSOs) induce skipping of CLN3 exon 5 and correct the CLN3Δ78 reading frame in CLN3Δ78/Δ78 mice; modified oligonucleotides (SSOs) distribute widely throughout the CNS following a single neonatal ICV injection (in mice). Modified oligonucleotide (SSO) reduces neuropathology in CLN3Δ78/Δ78 mice; Modified oligonucleotide (SSO) improves motor coordination of CLN3Δ78/Δ78 mice.

FIG. 24 provides a lay summary of experiments portrayed in FIGS. 1-24, and discussed herein. There is an urgent need to develop an effective treatment for CLN3 Batten disease, a fatal neurodegenerative disease affecting young children. In this study we have developed and tested a novel approach to therapeutically target the expression of the most common cause of the disease using small modified nucleic acid sequences directed to the mutated form of the gene with the aim of creating a method for treating Batten disease.

FIG. 25 provides a graph of mouse survival following treatment with a modified oligonucleotide complementary to CLN3 nucleic acid according to Example 8. Right side of the graph (120 days), from top to bottom, the lines represent data obtained from (1) CLN3+/+untreated mice; (2) CLN3+/Δ78 control modified oligonucleotide treated mice; (3) CLN3Δ78/Δ78 modified oligonucleotide (SSO-26) treated mice; (4) CLN3Δ78/Δ78 control modified oligonucleotide treated mice. Legend: from top to bottom (1) CLN3+/+ control untreated mice (n=33); (2) CLN3+/Δ78 modified oligonucleotide (control) treated mice (n=18); (3) CLN3Δ78/Δ78 modified oligonucleotide (control) treated mice (n=14); (4) CLN3Δ78/Δ78 modified oligonucleotide (SSO-26) treated mice (n=10).

FIG. 26 provides an overview of modified oligonucleotide-induced CLN3Δex 7/8 exon 5 skipping to correct the reading frame of CLN3 RNA. FIG. 26A provides a schematic showing an example of the correction of the CLN3Δ78 RNA reading frame. CLN3Δex 7/8 indicates CLN3 RNA lacking exons 7 and 8 (CLN3Δ78). CLN3Δex 5/7/8 indicates CLN3 RNA lacking exons 5, 7, and 8 following contact with a modified oligonucleotide (ASO-ex5). The expected length of the CLN3 protein translated from each of the three mRNAs is indicated to the right of the drawing. CLN3, CLN3Δex7/8 (Δ78), and the modified oligonucleotide-induced CLN3Δex5/7/8 (Δ578) spliced pre-mRNA isoforms. Modified oligonucleotide-induced skipping of exon 5 in CLN3Δex7/8 corrects the reading frame and eliminates the premature termination codon. Amino acids (aa) in the protein products are shown including the 28 aa frame-shifted residues preceding the stop codon in CLN3Δex7/8 and the 29 frame-shifted aa in CLN3Δex5/7/8 prior to frame-correction in exon 9. Exons are depicted as boxes, introns as lines, and splicing as diagonal lines. Stop codons and the regions encoding the lysosomal targeting signals (LTS) are labeled. FIG. 26B provides a schematic of human modified oligonucleotides (SSOs) #1-40 (corresponding to SEQ ID Nos: 57-90), each comprising a complementary sequence to the human CLN3 pre-mRNA exon 5 region and surrounding pre-mRNA introns. These modified oligonucleotides were assayed to identify those that induce the most CLN3 exon 5 skipping in human fibroblasts. The gray box indicates exon 5 and the bars the flanking introns. FIG. 26C provides two images of an acrylamide gel showing exon 5 skipping in CLN3+/Δ78 fibroblasts, as discussed in the legend to FIG. 16, and in Example 9. Radioactive RT-PCR was performed on RNA extracted from heterozygous hCLN3+/Δ7/8 fibroblast cells transfected with the indicated modified oligonucleotide. Quantification of the percent of exon 5 splicing in graph is calculated as: [Δ578/(Δ578+Δ78)]×100, and shown beneath the corresponding lane. A mock-treated control (M) is included. FIG. 26D provides an alignment of nucleobase sequences of SSO-20 (ASO-20) and SSO-28 (ASO-28) with the target hCLN3 region. The exonic sequence in capital letters and the intronic sequence in lower-case letters. FIG. 26E provides two images of RT-PCR analysis using RNA isolated from homozygous hCLN3Δex7/8 cells (CLN3Δ78/Δ78) treated with increasing doses of SSO-20 and SSO-28 (0 to 100 nM). The spliced products are indicated. The RT-PCR analysis was performed essentially as in Example 10, using the following primers: hCLN3ex4F (5′GCAACTCTGTCTCTACGGC-3′) (SEQ ID NO: 52) and hCLN3ex10R (5′CTTGAACACTGTCCACC-3′) (SEQ ID NO: 53). The graphs (right) represent the percent of exon 5 skipped in relationship to the log of the dose. The potency of the modified oligonucleotide was determined by calculating the half-maximal effective concentration (EC50) after fitting the data using nonlinear regression with a variable slope.

FIG. 27 provides the results of an assay of dose-dependent exon 5 skipping using human CLN3-directed modified oligonucleotides. FIG. 27A provides photos of the results of RT-PCR analysis using RNA isolated from a heterozygous CLN3+/Δex7/8 human fibroblast cell line treated with 3.125 to 200 nM of modified oligonucleotides SSO-20 (ASO-20) or SSO-28 (ASO-28). Spliced products are labeled. FIG. 27B provides graphs of the quantitation of exon 5 skipping, calculated as [exon 5 skipped products/(exon 5 included+exon 5 skipped)×100] (exon 5 skipped (%)), in relationship to the log of the dose and the half-maximal effective concentration (EC50).

FIG. 28 provides the results of an assay of dose -dependent exon 5 skipping using mouse CLN3-directed modified oligonucleotides in mouse cells. FIG. 28A provides a sequence alignment of SSO-26 (ASO-26) to the target CLN3 region. Cln3 exonic and intronic nucleotides are displayed as capital and lowercase letters, respectively. FIG. 28B provides photos of the results of RT-PCR analysis of exon 5 splicing from RNA extracted from homozygote mCln3Δex7/8 cells transfected with increasing concentrations of SSO-26 (0.391 nM to 200 nM). FIG. 28B provides graphs of the quantitation of exon 5 skipping, displaying the percent of exon 5 skipped in relationship to the log of the dose. The half-maximal effective concentration (EC50) was calculated after fitting the data using nonlinear regression, variable slope.

FIG. 29 provides an analysis of the exon 5-skipping activity of modified oligonucleotide S SO-26 in the CNS of treated mice. FIG. 29A provides images of RT-PCR analysis of RNA extracted from the cortex, thalamus, and striatum, and FIG. 19B provides images of RT-PCR analysis of RNA extracted from the brain stem, spinal cord, and kidney, of 19 week old Cln3+/Δex7/8 and Cln3Δex7/8/Δex7/8 mice treated at P1 or P2 with modified oligonucleotides SSO-C (control) or SSO-26. FIG. 29C and FIG. 29D provide bar graphs of the quantification of exon 5 skipping in the analysis of FIGS. 29A and 29B, respectively. Statistical significance was determined by one-way ANOVA with Dunnett's multiple comparisons test. *P<0.05, ****P<0.0001, n.s. not significant.

FIG. 30 provides bar graphs analyzing the weight of mice treated with modified oligonucleotide SSO-26 compared to control modified oligonucleotide SSO-C. FIG. 30A provides an analysis of weight of 2 month old male and female heterozygous and mutant mice treated at P1 or P2 with SSO-C or SSO-26. FIG. 30B provides an analysis of weight of 2 month old male and female heterozygous and mutant mice treated at P1 or P2 with SSO-C or SSO-26. Het denotes CLN3+/Δ78 mice, Mut denotes CLN3Δ78/Δ78 mice.

FIGS. 31A-31F provide the results of experiments indicating that modified oligonucleotide ASO-26 (SSO-26) treatment reduces subunit C of mitochondrial ATP synthase (SCMAS) in Cln3Δex7/8 mice. FIG. 31A: Immunofluorescent staining for SCMAS (green) and nuclei (stained with Hoechst; blue) in the hippocampus, thalamus, and cortex of 19 week old heterozygous mice treated with control ASO-C, homozygous Cln3Δex7/8 mice treated with ASO-C or ASO-26 at P1 or P2. For hippocampus and cortex N=7, 6, and 7 for Cln3+/Δex7/8 ASO-C, Δex7/8/Δex7/8 ASO-C, and Δex7/8/Δex7/8 ASO-26, respectively. For thalamus N=5. Scale bar, 100 μm. FIG. 31B Quantitative analysis of SCMAS in a. Columns and bars represent mean±s.e.m. Statistical significance was determined by one-way ANOVA with Dunnett's multiple comparisons test. *P<0.05, **P<0.01, ***P<0.001, ****P<0.0001. FIG. 31C: Photos of analysis of glial fibrillary acidic protein (GFAP) in the thalamus and cortex of 19 week-old Cln3+/Δex7/8 and Cln3 Δex7/8/Δex7/8 mice treated as neonates with either control ASO (ASO-C) or ASO-26. Scale bar, 100 μm. (below) FIG. 31D: Graphs of the quantitative analysis of GFAP accumulation in the corresponding regions, displayed as mean s.e.m. N=5-6 mice; n=45-64 image fields/mouse. Statistical significance was determined by one-way ANOVA with Dunnett's multiple comparisons test. *p<0.05, ***p<0.001, ****p<0.0001. FIG. 31E: Cln3Δex7/8/Δex7/8 mice treated with ASO-C or ASO-26 at P1 or 2, were assessed for motor activity in accelerating rotarod at 2 months of age. The latency to fall on the accelerating rotarod plotted as mean±s.e.m (left). N=39, 34, 31 for Cln3+/Δex7/8 ASO-C, Cln3Δex7/8/Δex7/8 ASO-C, and Cln3Δex7/8/Δex7/8 ASO-26 treated mice, respectively. FIG. 31F: Vertical pole test to assess motor coordination. The average time to turn downward 1800 on a vertical pole plotted as mean±s.e.m (right). N=19, 17, 19 for Cln3+/Δex7/8 ASO-C, Cln3Δex7/8/Δex7/8 ASO-C, and Cln3Δex7/8/Δex7/8 ASO-26 treated mice, respectively. Statistical significance was determined using one-way ANOVA and Tukey's multiple comparisons test. **P<0.01, ***P<0.001, ****P<0.0001.

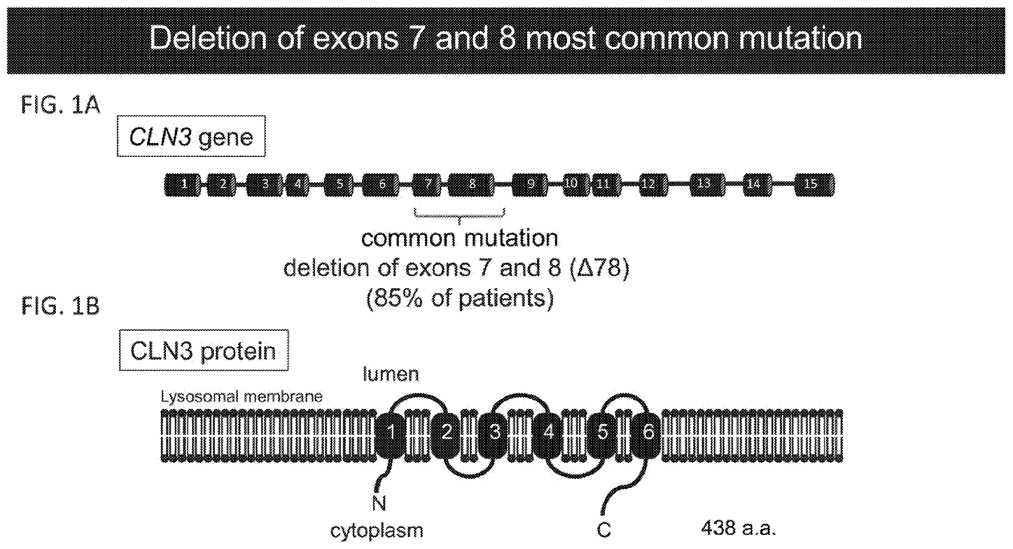

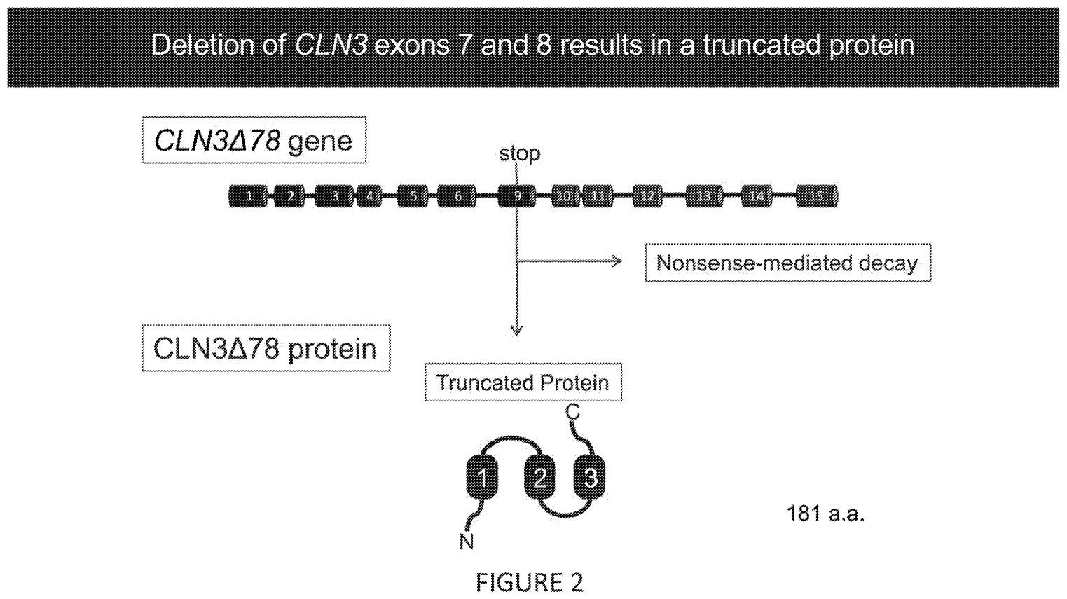

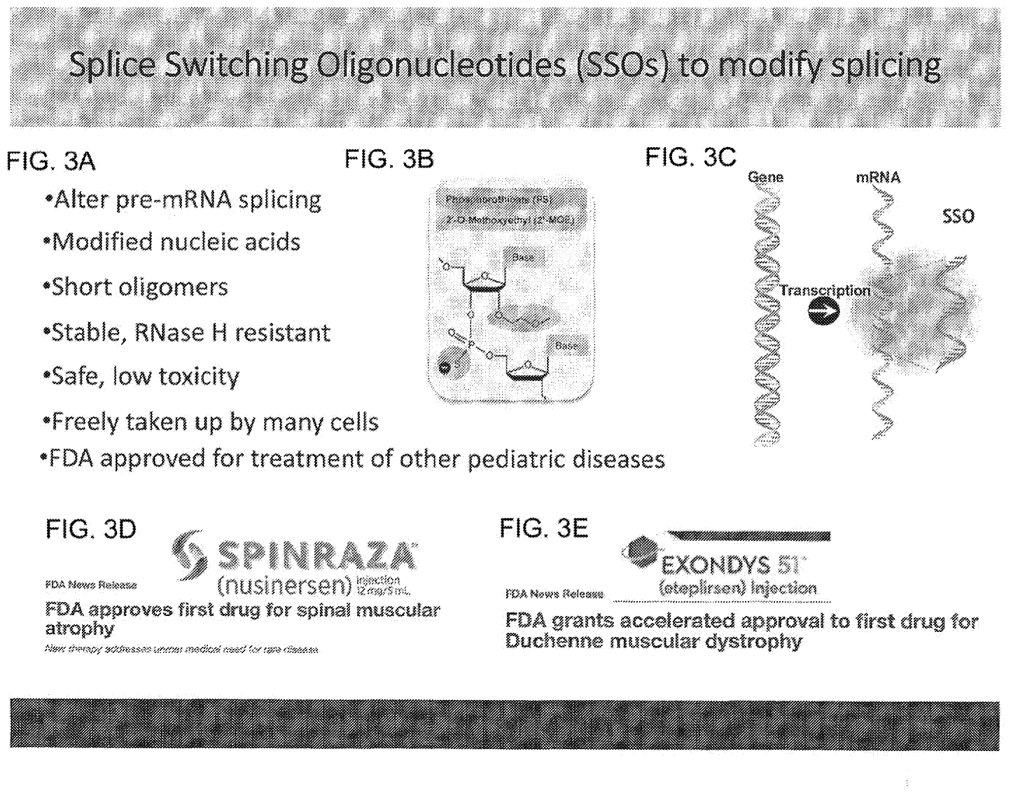

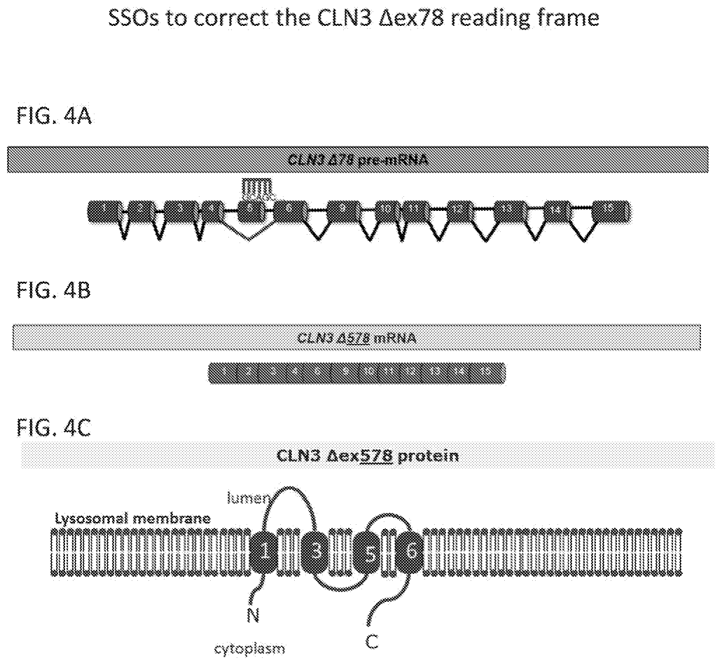

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxynucleoside” means a nucleoside comprising a 2′-H(H) deoxyribosy sugar moiety, as found in naturally occurring deoxyribonucleic acids (DNA). In certain embodiments, a 2′-deoxynucleoside may comprise a modified nucleobase or may comprise an RNA nucleobase (uracil).

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “5-methyl cytosine” means a cytosine modified with a methyl group attached to the 5 position. A 5-methyl cytosine is a modified nucleobase.

As used herein, “administering” means providing a pharmaceutical agent to an animal

As used herein, “animal” means a human or non-human animal

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense compound” means an oligomeric compound capable of achieving at least one antisense activity.

As used herein, “ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom. In certain embodiments, the symptom or hallmark is poor motor function/coordination, seizures, vision loss, poor cognitive function, psychiatric problems, accumulation of autofluorescent ceroid lipopigment in brain tissue, brain tissue dysfunction, brain tissue cell death, accumulation of mitochondrial ATP synthase subunit C in brain tissue, accumulation of lipofuscin in brain tissue, or astrocyte activation in brain tissue. In certain embodiments, amelioration of these symptoms results in improved motor function, reduced seizures, reduced vision loss or improvement of vision, improved cognitive function, reduced psychiatric problems, reduced accumulation of autofluorescent ceroid lipopigment in brain tissue, improved brain tissue function, reduced levels of brain tissue cell death, reduced accumulation of mitochondrial ATP synthase subunit C in brain tissue, reduced accumulation of lipofuscin in brain tissue, reduced astrocyte activation in brain tissue, and greater mean survival of treated animals or humans compared to untreated animals or humans

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “cleavable moiety” means a bond or group of atoms that is cleaved under physiological conditions, for example, inside a cell, an animal, or a human

As used herein, “CLN3 gene” means a gene that encodes a ceroid-lipofuscinosis, neuronal 3 protein and any ceroid-lipofuscinosis, neuronal 3 protein isoforms.

As used herein, “CLN3Δ78” means a CLN3 gene having a deletion spanning all or part of exons 7 and 8. In certain embodiments, the CLN3Δ78 deletion causes a frame-shift that result in a premature stop codon in exon 9. In certain embodiments, the truncated protein product of CLN3Δ78 is 33% of the length of the wild type.

As used herein, “complementary” in reference to an oligonucleotide means that at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include adenine (A) and thymine (T), adenine (A) and uracil (U), cytosine (C) and guanine (G), 5-methyl cytosine (mC) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

As used herein, “conjugate group” means a group of atoms that is directly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “constrained ethyl” or “cEt” or “cEt modified sugar” means a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′—CH(CH₃)—O-2′, and wherein the methyl group of the bridge is in the S configuration.

As used herein, “cEt nucleoside” means a nucleoside comprising a cEt modified sugar.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “exon 5 amino acids” means the portion of a CLN3 protein that corresponds to exon 5 of the CLN3 RNA. “Exon 10 amino acids” means the portion of a CLN3 protein that corresponds to exon 10 of the CLN3 RNA.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” Unless otherwise indicated, “gapmer” refers to a sugar motif. Unless otherwise indicated, the sugar moieties of the nucleosides of the gap of a gapmer are unmodified 2′-deoxyribosyl. Thus, the term “MOE gapmer” indicates a gapmer having a sugar motif of 2′-MOE nucleosides in both wings and a gap of 2′-deoxynucleosides. Unless otherwise indicated, a MOE gapmer may comprise one or more modified internucleoside linkages and/or modified nucleobases and such modifications do not necessarily follow the gapmer pattern of the sugar modifications.

As used herein, “hotspot region” is a range of nucleobases on a target nucleic acid amenable to oligomeric compound-mediated modulation of the amount or activity of the target nucleic acid.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, the term “internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate internucleoside linkage” is a modified internucleoside linkage in which one of the non-bridging oxygen atoms of a phosphodiester internucleoside linkage is replaced with a sulfur atom.

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “non-bicyclic modified sugar moiety” means a modified sugar moiety that comprises a modification, such as a substituent, that does not form a bridge between two atoms of the sugar to form a second ring.

As used herein, “mismatch” or “non-complementary” means a nucleobase of a first oligonucleotide that is not complementary with the corresponding nucleobase of a second oligonucleotide or target nucleic acid when the first and second oligonucleotide are aligned.

As used herein, “modulation” “modulate” or “modulating” means a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression. As a further example, modulating the expression of a RNA molecule can include a change in splice site selection of pre-mRNA processing, resulting in a change in the absolute or relative amount of a particular splice-variant compared to the amount in the absence of modulation. For example, modulating the expression of CLN3 RNA in a cell or animal can include a change in splice site selection of pre-mRNA processing, resulting in a change in the absolute or relative amount of a particular CLN3 splice-variant in a cell or animal relative to the absolute or relative amount of the particular CLN3 splice variant in an untreated or control sample cell or animal In further examples, modulating the expression of CLN3 RNA means an increase in the amount of CLN3 mRNA that lacks exon 5 in a treated sample cell, or animal, compared to the amount of CLN3 mRNA that lacks exon 5 in an untreated or control sample cell, or animal In further examples, modulating the expression of CLN3 RNA means an increase in the percentage of CLN3 mRNA that lacks exon 5 in a treated sample cell, or animal, compared to the percentage of CLN3 mRNA that lacks exon 5 in an untreated or control sample cell, or animal The percentage of CLN3 RNA that lacks exon 5 may be determined, for example, by calculating the percentage of CLN3 mRNA that lacks exon 5 over total CLN3 mRNA (CLN3 mRNA that includes exon 5 and CLN3 mRNA that lacks exon 5) in a cell, or animal For example, [Δ578/(Δ578+Δ78)]×100]. Further examples of modulating the expression of CLN3 RNA include modifying splicing, modifying CLN3 splicing, modifying the CLN3 RNA reading frame, for example correcting the CLN3Δex78 reading frame, promoting CLN3 exon 5 skipping, for example promoting CLN3 exon 5 skipping to restore the mRNA reading frame, skipping of CLN3 exon 5 in CLN3Δ78/Δ78 or +/Δ78 cells, splice-switching of CLN3 RNA, altering CLN3 pre-mRNA splicing, inducing exon 5 splicing.

Modulating the expression of CLN3 protein in a cell or animal means a change of amount or quality of CLN3 protein compared to the amount or quality of CLN3 protein prior to modulation. In further examples, modulating the expression of CLN3 protein means an increase in activity of CLN3 protein, or an increase in the amount of CLN3 protein that lacks exon 5 amino acids compared to the activity of CLN3 protein, or the amount of CLN3 protein that lacks exon 5 amino acids in an untreated or control sample cell or animal In further examples, modulating the expression of CLN3 protein means an increase in the percentage of CLN3 protein that lacks exon 5 amino acids in a cell, or animal, compared to the percentage of CLN3 protein in an untreated or control sample cell, or animal The percentage of CLN3 protein that lacks exon 5 amino acids may be determined, for example, by calculating the percentage of CLN3 protein that lacks exon 5 over total CLN3 protein (CLN3 protein that includes exon 5 and CLN3 protein that lacks exon 5) times 100, in a cell, or animal Or for example, the percentage of CLN3 protein that lacks exons 5, 7, and 8, over the total of CLN3 protein that lacks exon 7 and 8 and CLN3 protein that lacks exons 5, 7, and 8, times 100 [Δ578/(Δ578+Δ78)]×100]. In further examples, modulating the expression of CLN3 protein means an increase in activity of CLN3 protein, or an increase in the amount or percentage of CLN3 protein that includes exon 10, exon 11, exon 12, exon 13, exon 14, or exon 15 amino acids compared to the activity of CLN3 protein, or the amount or percentage of CLN3 protein that includes exon 10, exon 11, exon 12, exon 13, exon 14, or exon 15 amino acids in an untreated or control sample cell or animal. In calculating the amount or percentage of CLN3 protein that includes exon 10, exon 11, exon 12, exon 13, exon 14, or exon 15 amino acids, for heterozygous cells (e.g., CLN3+/45 cells), the wild type, or full length protein may also be considered. That is, for example, the percentage of CLN3 protein that includes exon 10 amino acids may be calculated as, for example, as [+ex10/(−ex10++ex10)]×100] (all CLN3 protein comprising exon 10 amino acids over CLN3 protein comprising exon 10 amino acids plus CLN3 protein lacking exon 10 amino acids). Alternatively, only the CLN3 protein that lacks exons 7 and 8 may be included in this calculation, for example, [+exon 10 aa Δ578/(+exon 10 aa Δ578+-exon 10Δ578+-exon 10Δ78)]×100] (all CLN3 protein comprising exon 10 amino acids and lacking exons 5, 7, and 8 over all CLN3 protein comprising exon 10 amino acids and lacking exons 5, 7, and 8 plus CLN3 protein lacking exon 10 amino acids and lacking exons 5, 7, and 8 plus CLN3 protein lacking exon 10 amino acids and lacking exons 7 and 8).

As used herein, “MOE” means methoxyethyl. “2′-MOE” or “2′-MOE modified sugar” means a 2′-OCH₂CH₂OCH₃ group in place of the 2′-OH group of a ribosyl sugar moiety. As used herein, “2′-MOE nucleoside” means a nucleoside comprising a 2′-MOE modified sugar.

As used herein, “motif” means the pattern of unmodified and/or modified sugar moieties, nucleobases, and/or internucleoside linkages, in an oligonucleotide.

As used herein, “neurodegenerative disease” means a condition marked by progressive loss of function or structure, including loss of motor function and death of neurons. In certain embodiments, the neurodegenerative disease is juvenile Batten disease, also known as juvenile neuronal ceroid lipofuscinosis (Batten Disease) and Batten disease.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase. A “5-methyl cytosine” is a modified nucleobase. A universal base is a modified nucleobase that can pair with any one of the five unmodified nucleobases. As used herein, “nucleobase sequence” means the order of contiguous nucleobases in a nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a contiguous sequence (i.e., no additional nucleosides are presented between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications. Modified oligonucleotides discussed herein include, for example, splice-switching antisense oligonucleotides, SSOs, splice switching oligonucleotides, ASOs, antisense oligonucleotides, therapeutic splice-switching antisense oligonucleotides, splice-skipping oligonucleotides, as, for example, discussed in the Examples and Description of Drawings herein.

As used herein, “pharmaceutically acceptable carrier or diluent” means any substance suitable for use in administering to an animal Certain such carriers enable pharmaceutical compositions to be formulated as, for example, tablets, pills, dragees, capsules, liquids, gels, syrups, slurries, suspension and lozenges for the oral ingestion by a subject.

In certain embodiments, a pharmaceutically acceptable carrier or diluent is sterile water, sterile saline, sterile buffer solution or sterile artificial cerebrospinal fluid.

As used herein “pharmaceutically acceptable salts” means physiologically and pharmaceutically acceptable salts of compounds Pharmaceutically acceptable salts retain the desired biological activity of the parent compound and do not impart undesired toxicological effects thereto.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein “prodrug” means a therapeutic agent in a form outside the body that is converted to a different form within an animal or cells thereof. Typically, conversion of a prodrug within the animal is facilitated by the action of an enzyme (e.g., endogenous or viral enzyme) or chemicals present in cells or tissues and/or by physiologic conditions.

As used herein, “RNA” means an RNA transcript and includes pre-mRNA and mature mRNA unless otherwise specified.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics In certain embodiments, an RNAi compound modulates the amount, activity, and/or splicing of a target nucleic acid. The term RNAi compound excludes antisense compounds that act through RNase H.

As used herein, “self-complementary” in reference to an oligonucleotide means an oligonucleotide that at least partially hybridizes to itself.

As used herein, “standard cell assay” means the assay described in Example 3 and reasonable variations thereof.

As used herein, “standard in vivo assay” means the experiment described in Example 7 and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the (5) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “sugar moiety” means an unmodified sugar moiety or a modified sugar moiety. As used herein, “unmodified sugar moiety” means a 2′—OH(H) ribosyl moiety, as found in RNA (an “unmodified RNA sugar moiety”), or a 2′—H(H) deoxyribosyl moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “target nucleic acid” and “target RNA” mean a nucleic acid that an antisense compound is designed to affect.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “therapeutically effective amount” means an amount of a pharmaceutical agent that provides a therapeutic benefit to an animal For example, a therapeutically effective amount improves a symptom of a disease.

### CERTAIN EMBODIMENTS

The present disclosure provides the following non-limiting numbered embodiments:
Embodiment 1. An oligomeric compound comprising a modified oligonucleotide consisting of 12 to 50 linked nucleosides wherein the nucleobase sequence of the modified oligonucleotide is at least 90% complementary to an equal length portion of a CLN3 nucleic acid, and wherein at least one nucleoside of the modified oligonucleotide comprises a modified sugar and/or at least one internucleoside linkage of the modified oligonucleotide is a modified internucleoside linkage.
Embodiment 2. An oligomeric compound comprising a modified oligonucleotide consisting of 12 to 50 linked nucleosides and having a nucleobase sequence comprising at least 12, at least 13, at least 14, at least 15, at least 16, at least 17, or 18 contiguous nucleobases of any of the nucleobase sequences of SEQ ID NOS: 57-96.
Embodiment 3. An oligomeric compound comprising a modified oligonucleotide consisting of 12 to 50 linked nucleosides and having a nucleobase sequence comprising a portion of at least 8, at least 9, at least 10, at least 11, at least 12, at least 13, at least 14, at least 15, at least 16, at least 17, at least 18, at least 19, or at least 20 contiguous nucleobases, wherein the portion is complementary to:
   an equal length portion of nucleobases 5499-5701 of SEQ ID NO: 1;
   an equal length portion of nucleobases 5514-5651 of SEQ ID NO: 1;
   an equal length portion of nucleobases 5519-5546 of SEQ ID NO: 1;
   an equal length portion of nucleobases 5534-5646 of SEQ ID NO: 1;
   an equal length portion of nucleobases 5559-5631 of SEQ ID NO: 1; or
   an equal length portion of nucleobases 5534-5551 of SEQ ID NO: 1.
Embodiment 4. The oligomeric compound of any of embodiments 1-3, wherein the modified oligonucleotide has a nucleobase sequence that is at least 80%, at least 85%, at least 90%, at least 95%, or 100% complementary to the nucleobase sequences of SEQ ID NO: 1 when measured across the entire nucleobase sequence of the modified oligonucleotide.
Embodiment 5. An oligomeric compound comprising a modified oligonucleotide consisting of 12 to 50 linked nucleosides and having a nucleobase sequence comprising at least 12, at least 13, at least 14, at least 15, at least 16, at least 17, or 18 contiguous nucleobases of any of the nucleobase sequences of SEQ ID NOS: 3-51.
Embodiment 6. An oligomeric compound comprising a modified oligonucleotide consisting of 12 to 50 linked nucleosides and having a nucleobase sequence comprising a portion of at least 8, at least 9, at least 10, at least 11, at least 12, at least 13, at least 14, at least 15, at least 16, at least 17, at least 18, at least 19, or at least 20 contiguous nucleobases, wherein the portion is complementary to:
   an equal length portion of nucleobases 4837-4964 of SEQ ID NO: 2;
   an equal length portion of nucleobases 4852-4954 of SEQ ID NO: 2;
   an equal length portion of nucleobases 4922-4954 of SEQ ID NO: 2;
   an equal length portion of nucleobases 4932-4949 of SEQ ID NO: 2;
   an equal length portion of nucleobases 4852-4954 of SEQ ID NO: 2;
   an equal length portion of nucleobases 4892-4954 of SEQ ID NO: 2; or
   an equal length portion of nucleobases 4892-4909 of SEQ ID NO: 2.
Embodiment 7. The oligomeric compound of any of embodiments 1, 5, or 6, wherein the modified oligonucleotide has a nucleobase sequence that is at least 80%, at least 85%, at least 90%, at least 95%, or 100% complementary to the nucleobase sequences of SEQ ID NO: 2 when measured across the entire nucleobase sequence of the modified oligonucleotide.
Embodiment 8. The oligomeric compound of any of embodiments 1-7, wherein the modified oligonucleotide comprises at least one modified nucleoside.
Embodiment 9. The oligomeric compound of embodiment 8, wherein the modified oligonucleotide comprises at least one modified nucleoside comprising a modified sugar moiety.
Embodiment 10. The oligomeric compound of any one of embodiments 1-9, wherein the modified oligonucleotide comprises at least 1, at least 2, at least 3, at least 4, at least 5, at least 6, at least 7, at least 8, at least 9, at least 10, at least 11, at least 12, at least 13, at least 14, at least 15, at least 16, at least 17, or at least 18 modified nucleosides comprising a modified sugar moiety.
Embodiment 11. The oligomeric compound of any of embodiments 9 or 10, wherein the modified oligonucleotide comprises at least one modified nucleoside comprising a bicyclic sugar moiety.
Embodiment 12. The oligomeric compound of embodiment 11, wherein the modified oligonucleotide comprises at least 1, at least 2, at least 3, at least 4, at least 5, at least 6, at least 7, at least 8, at least 9, at least 10, at least 11, at least 12, at least 13, at least 14, at least 15, at least 16, at least 17, or at least 18 modified nucleosides comprising a bicyclic sugar moiety.
Embodiment 13. The oligomeric compound of any of embodiments 11 or 12, wherein the modified oligonucleotide comprises at least one modified nucleoside comprising a bicyclic sugar moiety having a 2′-4′ bridge, wherein the 2′-4′ bridge is selected from —O—CH₂—; and —O—CH(CH₃)—.
Embodiment 14. The oligomeric compound of embodiment 9, wherein the modified oligonucleotide comprises at least one modified nucleoside comprising a non-bicyclic modified sugar moiety.
Embodiment 15. The oligomeric compound of any of embodiments 9 or 10, wherein the modified oligonucleotide comprises at least 1, at least 2, at least 3, at least 4, at least 5, at least 6, at least 7, at least 8, at least 9, at least 10, at least 11, at least 12, at least 13, at least 14, at least 15, at least 16, at least 17, or at least 18 modified nucleosides comprising a non-bicyclic sugar moiety.
Embodiment 16. The oligomeric compound of any of embodiments 14 or 15, wherein the modified oligonucleotide comprises at least one modified nucleoside comprising a non-bicyclic modified sugar moiety comprising a 2′-MOE modified sugar or 2′-OMe modified sugar.
Embodiment 17. The oligomeric compound of embodiment 9, wherein each modified nucleoside of the modified oligonucleotide comprises a modified non-bicyclic sugar moiety comprising a 2′-MOE or 2′-OMe.
Embodiment 18. The oligomeric compound of embodiment 9 , wherein each modified nucleoside of the modified oligonucleotide comprises a modified non-bicyclic sugar moiety comprising 2′-MOE.
Embodiment 19. The oligomeric compound of any of embodiments 1-9, wherein the modified oligonucleotide comprises a fully-modified sugar motif region. Embodiment 20. The oligomeric compound of embodiment 19, wherein the fully-modified sugar motif region is 7 to 20 nucleosides in length.
Embodiment 21. The oligomeric compound of any of embodiments 19-20, wherein the modified oligonucleotide comprises at least 1, at least 2, at least 3, or at least 4 2′-deoxynucleosides.
Embodiment 22. The oligomeric compound of any of embodiments 19-21, wherein each nucleoside of the fully modified sugar motif region comprises a 2′-OCH₂CH₂OCH₃ group or a 2′-OCH₃ group.
Embodiment 23. The oligomeric compound of any of embodiments 8-18, wherein the modified oligonucleotide comprises at least one modified nucleoside comprising a sugar surrogate.
Embodiment 24. The oligomeric compound of embodiment 23, wherein the modified oligonucleotide comprises at least one modified nucleoside comprising a sugar surrogate selected from morpholino and PNA.
Embodiment 25. The oligomeric compound of any of embodiments 1-24, wherein the modified oligonucleotide comprises at least one modified internucleoside linkage.
Embodiment 26. The oligomeric compound of embodiment 25, wherein each internucleoside linkage of the modified oligonucleotide is a modified internucleoside linkage.
Embodiment 27. The oligomeric compound of embodiment 25 or 26 wherein at least one internucleoside linkage is a phosphorothioate internucleoside linkage.
Embodiment 28. The oligomeric compound of embodiment 25 or 27 wherein the modified oligonucleotide comprises at least one phosphodiester internucleoside linkage.

Embodiment 29. The oligomeric compound of any of embodiments 25, 27, or 28, wherein each internucleoside linkage is either a phosphodiester internucleoside linkage or a phosphorothioate internucleoside linkage.
Embodiment 30. The oligomeric compound of embodiment 26, wherein each internucleoside linkage of the modified oligonucleotide is a phosphorothioate internucleoside linkage.
Embodiment 31. The oligomeric compound of any of embodiments 1-7, wherein each modified nucleoside of the modified oligonucleotide comprises a modified non-bicyclic sugar moiety comprising 2′-MOE, and each modified internucleoside linkage of the modified oligonucleotide is a phosphorothioate internucleoside linkage.
Embodiment 32. The oligomeric compound of any of embodiments 1-31, wherein the modified oligonucleotide comprises at least one modified nucleobase.
Embodiment 33. The oligomeric compound of embodiment 32, wherein the modified nucleobase is a 5-methyl cytosine.

Embodiment 34. The oligomeric compound of embodiment 32, wherein the modified oligonucleotide comprises one or more cytosine nucleobases and each cytosine nucleobase is a 5-methyl cytosine.
Embodiment 35. The oligomeric compound of any of embodiments 1-34, wherein the nucleobase sequence of the modified oligonucleotide is at least 95% complementary to an equal length portion of a human CLN3 nucleic acid.
Embodiment 36. The oligomeric compound of any one of embodiments 1-35, wherein the modified oligonucleotide consists of 12-18, 12-20, 14-20, 16-20, or 17-19 linked nucleosides.
Embodiment 37. The oligomeric compound of any one of embodiments 1-35, wherein the modified oligonucleotide consists of 18 linked nucleosides.
Embodiment 38. The oligomeric compound of any of embodiments 1-35, wherein the modified oligonucleotide consists of 18 or 20 linked nucleosides.
Embodiment 39. The oligomeric compound of any of embodiments 1-38 consisting of the modified oligonucleotide.
Embodiment 40. The oligomeric compound of any of embodiments 1-38 comprising a conjugate group comprising a conjugate moiety and a conjugate linker.
Embodiment 41. The oligomeric compound of embodiment 40, wherein the conjugate group comprises a GalNAc cluster comprising 1-3 GalNAc ligands
Embodiment 42. The oligomeric compound of embodiments 40 or 41, wherein the conjugate linker consists of a single bond.
Embodiment 43. The oligomeric compound of embodiment 40, wherein the conjugate linker is cleavable.
Embodiment 44. The oligomeric compound of embodiment 42, wherein the conjugate linker comprises 1-3 linker-nucleosides.
Embodiment 45. The oligomeric compound of any of embodiments 41-44, wherein the conjugate group is attached to the modified oligonucleotide at the 5′-end of the modified oligonucleotide.
Embodiment 46. The oligomeric compound of any of embodiments 41-44, wherein the conjugate group is attached to the modified oligonucleotide at the 3′-end of the modified oligonucleotide.
Embodiment 47. The oligomeric compound of any of embodiments 1-46 comprising a terminal group.
Embodiment 48. The oligomeric compound of any of embodiments 1-47 wherein the oligomeric compound is a singled-stranded oligomeric compound.
Embodiment 49. The oligomeric compound of any of embodiments 1-42 or 44-48, wherein the oligomeric compound does not comprise linker-nucleosides.
Embodiment 50. An oligomeric duplex comprising an oligomeric compound of any of embodiments 47 or 49.
Embodiment 51. An antisense compound comprising or consisting of an oligomeric compound of any of embodiments 1-49 or an oligomeric duplex of embodiment 50.
Embodiment 52. The oligomeric compound of any of embodiments 1-7, wherein the modified oligonucleotide is an RNAi compound.
Embodiment 53. The oligomeric compound of embodiment 52, wherein the RNAi compound is an ssRNA or an siRNA.
Embodiment 54. A pharmaceutical composition comprising an oligomeric compound of any of embodiments 1-49 or embodiments 52-53, or an oligomeric duplex of embodiment 50, and a pharmaceutically acceptable carrier or diluent.
Embodiment 55. The pharm composition of embodiment 54, wherein the pharmaceutically acceptable diluent is phosphate-buffered saline (PBS).
Embodiment 56. The pharm composition of embodiment 54, wherein the modified oligonucleotide of the oligomeric compound or oligomeric duplex is a salt.
Embodiment 57. The pharm composition of embodiment 55, wherein the modified oligonucleotide of the oligomeric compound or oligomeric duplex is a salt.
Embodiment 58. The pharm composition of embodiment 56 or embodiment 57, wherein the salt is a sodium salt.
Embodiment 59. A chirally enriched population of the modified oligonucleotide of any of embodiments 1-50, wherein the modified oligonucleotide comprises at least one phosphorothioate internucleoside linkage and wherein the population is enriched for modified oligonucleotides comprising at least one particular phosphorothioate internucleoside linkage having a particular stereochemical configuration.
Embodiment 60. The chirally enriched population of embodiment 59, wherein the population is enriched for modified oligonucleotides comprising at least one particular phosphorothioate internucleoside linkage having the (Sp) configuration.
Embodiment 61. The chirally enriched population of embodiment 59 or 60, wherein the population is enriched for modified oligonucleotides comprising at least one particular phosphorothioate internucleoside linkage having the (Rp) configuration.
Embodiment 62. The chirally enriched population of embodiment 59, wherein the population is enriched for modified oligonucleotides having a particular, independently selected stereochemical configuration at each phosphorothioate internucleoside linkage
Embodiment 63. The chirally enriched population of embodiment 62, wherein the population is enriched for modified oligonucleotides having the (Sp) configuration at each phosphorothioate internucleoside linkage.
Embodiment 64. The chirally enriched population of embodiment 62, wherein the population is enriched for modified oligonucleotides having the (Rp) configuration at each phosphorothioate internucleoside linkage.
Embodiment 65. The chirally enriched population of embodiment 59 or embodiment 62 wherein the population is enriched for modified oligonucleotides having at least 3 contiguous phosphorothioate internucleoside linkages in the Sp-Sp-Rp configuration, in the 5′ to 3′ direction.
Embodiment 66. A population of modified oligonucleotides of any of embodiments 1-50, wherein the modified oligonucleotide comprises at least one phosphorothioate internucleoside linkage and wherein all of the phosphorothioate internucleoside linkages of the modified oligonucleotide are stereorandom.
Embodiment 67. A pharmaceutical composition comprising the chirally enriched population of any of embodiments 59-65 or the population of modified oligonucleotides of embodiment 66, and a pharmaceutically acceptable diluent or carrier.
Embodiment 68. The pharmaceutical composition of embodiments 54 or 67, wherein the pharmaceutically acceptable diluent is artificial cerebrospinal fluid.
Embodiment 69. The pharmaceutical composition of embodiment 68, wherein the pharmaceutical composition consists essentially of the modified oligonucleotide and artificial cerebrospinal fluid.
Embodiment 70. A method comprising administering to an animal a pharmaceutical composition of any of embodiments 54 or 67-69.
Embodiment 71. A method of treating a disease associated with CLN3 comprising administering to an individual having or at risk for developing a disease associated with CLN3 a therapeutically effective amount of a pharmaceutical composition according to any of embodiments 54 or 67-69; and thereby treating the disease associated with CLN3.
Embodiment 72. The method of embodiment 71, wherein the disease associated with CLN3 is a neurodegenerative disease.
Embodiment 73. The method of embodiment 72, wherein the neurodegenerative disease is Batten Disease.
Embodiment 74. The method of embodiment 73, wherein at least one symptom or hallmark of the neurodegenerative disease is ameliorated.
Embodiment 75. The method of embodiment 74, wherein the symptom or hallmark is poor motor function, seizures, vision loss, poor cognitive function, psychiatric problems, accumulation of autofluorescent ceroid lipopigment in brain tissue, brain tissue dysfunction, brain tissue cell death, accumulation of mitochondrial ATP synthase subunit C in brain tissue, accumulation of lipofuscin in brain tissue, or astrocyte activation in brain tissue.
Embodiment 76. The method of embodiment 75, wherein the brain tissue is the somatosensory cortex, visual cortex, thalamus, or hippocampus.
Embodiment 77. The oligomeric compound of any of embodiments 1-53, wherein the oligomeric compound induces CLN3 exon 5 skipping in vitro.
Embodiment 78. A method of modulating the expression of CLN3 in a cell, comprising contacting the cell with an oligomeric compound of any of embodiments 1-53; and thereby modulating expression of CLN3 in the cell.
Embodiment 79. A method of modulating splicing of CLN3 RNA in a cell, comprising contacting the cell with an oligomeric compound of any of embodiments 1-53; and thereby modulating splicing of CLN3 in the cell.
Embodiment 80. A method of inducing CLN3 exon 5 skipping in a cell, comprising contacting the cell with an oligomeric compound of any of embodiments 1-53; and thereby inducing CLN3 exon 5 skipping in the cell.
Embodiment 81. The method of any of embodiments 78-80, wherein the cell is a human cell.
Embodiment 82. The method of any of embodiments 78-81, wherein the amount of CLN3 mRNA molecules that comprises exon 5 in the cell is reduced compared to the amount prior to contacting the cell with the oligomeric compound; or the percentage of CLN3 mRNA molecules that comprises exon 5 in the cell is reduced compared to the percent prior to contacting the cell with the oligomeric compound.
Embodiment 83. The method of any of embodiments 78-81, wherein the amount of CLN3 mRNA molecules that comprises exon 5 in the cell is reduced compared to cells that have not been contacted with the oligomeric compound; or the percentage of CLN3 mRNA molecules that comprises exon 5 in the cell is reduced compared to cells that have not been contacted with the oligomeric compound.
Embodiment 84. The method of any of embodiments 78-81, wherein the amount of CLN3 protein comprising exon 10 amino acids in the cell increases compared to the amount prior to contacting the cell with the oligomeric compound; or the percentage of CLN3 protein molecules that comprises exon 10 amino acids in the cell increases compared to the percent prior to contacting the cell with the oligomeric compound.
Embodiment 85. The method of any of embodiments 78-81, wherein the amount of CLN3 protein comprising exon 10 amino acids in the cell increases compared to cells that have not been contacted with the oligomeric compound; or the percentage of CLN3 protein molecules that comprises exon 10 amino acids in the cell increases compared to cells that have not been contacted with the oligomeric compound.
Embodiment 86. A compound comprising or consisting of an oligonucleotide having a nucleobase sequence complementary to an equal length portion of a target region of a CLN3 nucleic acid, wherein the compound is capable of inducing skipping of CLN3 exon 5.
Embodiment 87. A compound comprising or consisting of an oligonucleotide consisting of 15 to 25 linked nucleosides and having a nucleobase sequence complementary to an equal length portion of a target region of a CLN3 nucleic acid, wherein the oligonucleotide comprises at least one 2′-O-methoxyethyl sugar moiety and/or at least one phosphorothioate internucleoside linkage.
Embodiment 88. A compound comprising or consisting of an oligonucleotide consisting of 18 linked nucleosides and having a nucleobase sequence complementary to an equal length portion of a target region of a human CLN3 nucleic acid, wherein the target region of the CLN3 nucleic acid is exon 5, intron 4 and/or intron 5 and wherein the compound is capable of inducing skipping of CLN3 exon 5.
Embodiment 89. A compound comprising or consisting of an oligonucleotide consisting of 18 linked nucleosides and having a nucleobase sequence of any one of SEQ ID NOs 57-96, wherein the compound is capable of inducing skipping of CLN3 exon 5.
Embodiment 90. A compound comprising or consisting of an oligonucleotide consisting of 18 linked nucleosides and having a nucleobase sequence complementary to an equal length portion of a target region of a human CLN3 nucleic acid, wherein the target region of the CLN3 nucleic acid is exon 5, intron 4 and/or intron 5 and wherein the oligonucleotide comprises at least one 2′-O-methoxyethyl sugar moiety and/or at least one phosphorothioate internucleoside linkage.
Embodiment 91. A compound comprising or consisting of an oligonucleotide consisting of 18 linked nucleosides and having a nucleobase sequence complementary to an equal length portion of a target region of a human CLN3 nucleic acid, wherein the target region of the CLN3 nucleic acid is exon 5, intron 4 or intron 5 and wherein each nucleoside comprises a 2′-O-methoxyethyl sugar moiety and/or each internucleoside linkage is a phosphorothioate internucleoside linkage.
Embodiment 92. A compound comprising or consisting of an oligonucleotide consisting of 18 linked nucleosides and having a nucleobase sequence of any one of SEQ ID NOs 57-96, and wherein the oligonucleotide comprises at least one 2′-O-methoxyethyl sugar moiety and/or at least one phosphorothioate internucleoside linkage.
Embodiment 93. A compound comprising or consisting of an oligonucleotide consisting of 18 linked nucleosides and having a nucleobase sequence of any one of SEQ ID NOs 57-96, and wherein each nucleoside comprises a 2′-O-methoxyethyl sugar moiety and/or each internucleoside linkage is a phosphorothioate internucleoside linkage.
Embodiment 94. A compound comprising or consisting of an oligonucleotide consisting of 18 linked nucleosides and having a nucleobase sequence complementary to an equal length portion of a target region of a human CLN3 nucleic acid, wherein the target region of the CLN3 nucleic acid is exon 5 and wherein each nucleoside comprises a 2′-O-methoxyethyl sugar moiety and each internucleoside linkage is a phosphorothioate internucleoside linkage.
Embodiment 95. A compound comprising or consisting of an oligonucleotide consisting of 18 linked nucleosides and having a nucleobase sequence complementary to an equal length portion of a target region of a human CLN3 nucleic acid, wherein the target region of the CLN3 nucleic acid is intron 4 and wherein each nucleoside comprises a 2′-O-methoxyethyl sugar moiety and each internucleoside linkage is a phosphorothioate internucleoside linkage.
Embodiment 96. A compound comprising or consisting of an oligonucleotide consisting of 18 linked nucleosides and having a nucleobase sequence complementary to an equal length portion of a target region of a human CLN3 nucleic acid, wherein the target region of the CLN3 nucleic acid is intron 5 and wherein each nucleoside comprises a 2′-O-methoxyethyl sugar moiety and each internucleoside linkage is a phosphorothioate internucleoside linkage.
Embodiment 97. A compound comprising or consisting of an oligonucleotide consisting of 18 linked nucleosides and having a nucleobase sequence complementary to an equal length portion of a target region of a human CLN3 nucleic acid, wherein the target region of the CLN3 nucleic acid spans intron 4 and exon 5 and wherein each nucleoside comprises a 2′-O-methoxyethyl sugar moiety and each internucleoside linkage is a phosphorothioate internucleoside linkage.
Embodiment 98. A compound comprising or consisting of an oligonucleotide consisting of 18 linked nucleosides and having a nucleobase sequence complementary to an equal length portion of a target region of a human CLN3 nucleic acid, wherein the target region of the CLN3 nucleic acid spans exon 5 and intron 5 and wherein each nucleoside comprises a 2′-O-methoxyethyl sugar moiety and each internucleoside linkage is a phosphorothioate internucleoside linkage.
Embodiment 99. A compound comprising or consisting of an oligonucleotide consisting of 18 linked nucleosides and having a nucleobase sequence of any one of SEQ ID NOs 57-96, and wherein each nucleoside comprises a 2′-O-methoxyethyl sugar moiety and each internucleoside linkage is a phosphorothioate internucleoside linkage.
Embodiment 100.A pharmaceutical composition comprising a compound according to any one of embodiments 86-99.
Embodiment 101.A compound according to any one of embodiments 86-99 or a pharmaceutical composition according to embodiment 100, for use in therapy.
Embodiment 102.A compound according to any one of embodiments 86-99 or a pharmaceutical composition according to embodiment 100, for use in treating juvenile Batten Disease in a subject wherein optionally the compound is capable of improving motor coordination and/or reducing neuropathy in the subject.
Embodiment 103.The compound or the pharmaceutical composition of embodiment 102, wherein the Batten Disease is juvenile Batten Disease.
Embodiment 104.Use of a compound according to any one of embodiments 86-99, or a pharmaceutical composition according to embodiment 100, in the manufacture of a medicament.
Embodiment 105.Use of a compound according to any one of embodiments 86-99, or a pharmaceutical composition according to embodiment 100, in the manufacture of a medicament for treating Batten Disease.

Embodiment 106.The oligomeric compound according to any one of embodiments 1-7, wherein each modified nucleoside of the modified oligonucleotide comprises a modified non-bicyclic sugar moiety comprising 2′-MOE, each modified internucleoside linkage of the modified oligonucleotide is a phosphorothioate internucleoside linkage, and each cytosine nucleobase is a 5-methyl cytosine.
Embodiment 107.The oligomeric compound of embodiment 1, wherein the modified oligonucleotide consists of 18 linked nucleosides; each modified nucleoside of the modified oligonucleotide comprises a modified non-bicyclic sugar moiety comprising 2′-MOE;
   each modified internucleoside linkage of the modified oligonucleotide is a phosphorothioate internucleoside linkage; and
   each cytosine nucleobase is a 5-methyl cytosine; and
   wherein the modified oligonucleotide has a nucleobase sequence complementary to:
      an equal length portion of nucleobases 5499-5701 of SEQ ID NO: 1;
      an equal length portion of nucleobases 5514-5651 of SEQ ID NO: 1;
      an equal length portion of nucleobases 5519-5546 of SEQ ID NO: 1;
      an equal length portion of nucleobases 5534-5646 of SEQ ID NO: 1;
      an equal length portion of nucleobases 5559-5631 of SEQ ID NO: 1; or
      an equal length portion of nucleobases 5534-5551 of SEQ ID NO: 1.
Embodiment 108.The oligomeric compound according to any one of embodiments 1-3, wherein the modified oligonucleotide has a nucleobase sequence that is at least 90% complementary to the nucleobase sequence of SEQ ID NO: 1 when measured across the entire nucleobase sequence of the modified oligonucleotide, wherein
   each modified nucleoside of the modified oligonucleotide comprises a modified non-bicyclic sugar moiety comprising 2′-MOE;
   each modified internucleoside linkage of the modified oligonucleotide is a phosphorothioate internucleoside linkage; and
   each cytosine nucleobase is a 5-methyl cytosine.
Embodiment 109.An oligomeric compound comprising a modified oligonucleotide consisting of 18 or 20 linked nucleosides wherein the nucleobase sequence of the modified oligonucleotide is at least 90% complementary to an equal length portion of a CLN3 nucleic acid, wherein
   each modified nucleoside of the modified oligonucleotide comprises a modified non-bicyclic sugar moiety comprising 2′-MOE;
   each modified internucleoside linkage of the modified oligonucleotide is a phosphorothioate internucleoside linkage; and
   each cytosine nucleobase is a 5-methyl cytosine.
Embodiment 110.An oligomeric compound comprising a modified oligonucleotide consisting of 12 to 50 linked nucleosides and having a nucleobase sequence comprising at least 12, at least 13, at least 14, at least 15, at least 16, at least 17, or 18 contiguous nucleobases of any of the nucleobase sequences of SEQ ID NOS: 57-96, wherein
   each modified nucleoside of the modified oligonucleotide comprises a modified non-bicyclic sugar moiety comprising 2′-MOE;
   each modified internucleoside linkage of the modified oligonucleotide is a phosphorothioate internucleoside linkage; and
   each cytosine nucleobase is a 5-methyl cytosine.
Embodiment 111.An oligomeric compound comprising a modified oligonucleotide consisting of 12 to 50 linked nucleosides and having a nucleobase sequence comprising at least 12, at least 13, at least 14, at least 15, at least 16, at least 17, or 18 contiguous nucleobases of any of the nucleobase sequences of SEQ ID NOS: 57-96, wherein
   the modified oligonucleotide has a nucleobase sequence that is at least 90% complementary to the nucleobase sequences of SEQ ID NO: 1 when measured across the entire nucleobase sequence of the modified oligonucleotide;
   each modified nucleoside of the modified oligonucleotide comprises a modified non-bicyclic sugar moiety comprising 2′-MOE;
   each modified internucleoside linkage of the modified oligonucleotide is a phosphorothioate internucleoside linkage; and
   each cytosine nucleobase is a 5-methyl cytosine.
Embodiment 112.An oligomeric compound comprising a modified oligonucleotide consisting of 18 linked nucleosides and having a nucleobase sequence of any of the nucleobase sequences of SEQ ID NOS: 57-96, wherein
   each modified nucleoside of the modified oligonucleotide comprises a modified non-bicyclic sugar moiety comprising 2′-MOE;
   each modified internucleoside linkage of the modified oligonucleotide is a phosphorothioate internucleoside linkage; and
   each cytosine nucleobase is a 5-methyl cytosine.
Embodiment 113.The oligomeric compound according to any one of embodiments 106-112, wherein the oligomeric compound is capable of inducing skipping of CLN3 exon 5.
Embodiment 114.A pharmaceutical composition comprising an oligomeric compound of according to any one of embodiments 106-113, and a pharmaceutically acceptable carrier or diluent. Embodiment 115.The pharmaceutical composition of embodiment 100, comprising a pharmaceutically acceptable diluent.
Embodiment 116.The pharm composition according to any one of embodiments 114 or 115, wherein the pharmaceutically acceptable diluent is phosphate-buffered saline (PBS).
Embodiment 117.The pharm composition according to any one of embodiments 100, or 114-115, wherein the modified oligonucleotide of the oligomeric compound or oligomeric duplex is a salt.
Embodiment 118.The pharm composition of embodiment 117, wherein the salt is a sodium salt.
Embodiment 119.An oligomeric compound according to any one of embodiments 1-49 or 106-113 or a pharmaceutical composition according to any one of embodiments 67-69 or 114-118, for use in therapy.
Embodiment 120 Use of a compound according to any one of embodiments 1-49 or 106-113, or a pharmaceutical composition according to any of embodiments 67-69 or 114-118, in the manufacture of a medicament. Embodiment 121. Use of a compound according to any one of embodiments 1-49 or 106-113, or a pharmaceutical composition according to any of embodiments 67-69 or 114-118, in the manufacture of a medicament for treating Batten Disease.
Embodiment 122.A chirally enriched population according to any one of embodiments 59-65, a population of modified oligonucleotides according to embodiment 66, or a pharmaceutical composition according to any of embodiments 67-69, for use in therapy.
Embodiment 123.Use of a chirally enriched population according to any one of embodiments 59-65, a population of modified oligonucleotides according to embodiment 66, or a pharmaceutical composition according to any of embodiments 67-69, in the manufacture of a medicament.
Embodiment 124.Use of a chirally enriched population according to any one of embodiments 59-65, a population of modified oligonucleotides according to embodiment 66, or a pharmaceutical composition according to any of embodiments 67-69, in the manufacture of a medicament for treating Batten Disease.
Embodiment 125. A method of treating a disease associated with CLN3 comprising administering to an individual having or at risk for developing a disease associated with CLN3 a therapeutically effective amount of a pharmaceutical composition according to any one of embodiments 110, or 114-118; and thereby treating the disease associated with CLN3.
Embodiment 126. The method of embodiment 125, wherein the disease associated with CLN3 is a neurodegenerative disease.
Embodiment 127. The method of embodiment 126, wherein the neurodegenerative disease is Batten Disease.
Embodiment 128. The method of embodiment 127, wherein at least one symptom or hallmark of the neurodegenerative disease is ameliorated.
Embodiment 129.The method of embodiment 128, wherein the symptom or hallmark is poor motor function, seizures, vision loss, poor cognitive function, psychiatric problems, accumulation of autofluorescent ceroid lipopigment in brain tissue, brain tissue dysfunction, brain tissue cell death, accumulation of mitochondrial ATP synthase subunit C in brain tissue, accumulation of lipofuscin in brain tissue, or astrocyte activation in brain tissue.
Embodiment 130.The method of embodiment 129, wherein the brain tissue is the somatosensory cortex, visual cortex, thalamus, or hippocampus.
Embodiment 131.The oligomeric compound according to any one of embodiments 106-113 wherein the compound induces CLN3 exon 5 skipping in vitro.
Embodiment 132. A method of modulating the expression of CLN3 in a cell, comprising contacting the cell with an oligomeric compound according to any one of embodiments 106-113 or a compound of any one of embodiments 86-99;
and thereby modulating expression of CLN3 in the cell. Embodiment 133.A method of modulating splicing of CLN3 RNA in a cell, comprising contacting the cell with an oligomeric compound according to any one of embodiments 106-113 or a compound of any one of embodiments 86-99; and thereby modulating splicing of CLN3 in the cell.
Embodiment 134.A method of inducing CLN3 exon 5 skipping in a cell, comprising contacting the cell with an oligomeric compound according to any one of embodiments 106-113 or a compound of any one of embodiments 86-99; and thereby inducing CLN3 exon 5 skipping in the cell.
Embodiment 135.The method of according to any one of embodiments 132-134, wherein the cell is a human cell.
Embodiment 136.The method according to any one of embodiments 132-134, wherein
   the amount of CLN3 mRNA molecules that comprise exon 5 in the cell is reduced compared to the amount prior to contacting the cell with the compound; or
   the percentage of CLN3 mRNA molecules that comprise exon 5 in the cell is reduced compared to the percent prior to contacting the cell with the compound.
Embodiment 137.The method according to any one of embodiments 132-134, wherein
   the amount of CLN3 protein comprising exon 10 amino acids in the cell increases compared to the amount prior to contacting the cell with the compound;
   or the percentage of CLN3 protein molecules that comprise exon 10 amino acids in the cell increases compared to the percent prior to contacting the cell with the compound.

### I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

### A. Certain Modified Nucleosides

Modified nucleosides comprise a modified sugar moiety or a modified nucleobase or both a modified sugar moiety and a modified nucleobase.

### 1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O (CH₂)₂OCH₃ (“2′-MOE”). In certain embodiments, 2′-substituent groups are selected from among: halo, allyl, amino, azido, SH, CN, OCN, CF₃, OCF₃, O-C₁-C₁₀ alkoxy, O-C₁-C₁₀ substituted alkoxy, O-C₁-C₁₀ alkyl, O-C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂O N(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted non-bicyclic modified nucleoside comprises a sugar moiety comprising a non-bridging 2′-substituent group selected from: F, NH₂, N3, OCF₃, OCH₃, O(CH₂)₃NH₂, CH2CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH2)3-2′, 4′-CH₂-O-2′ (“LNA”), 4′-CH₂—S—2′, 4′-(CH₂)₂—O-2′ (“ENA”), 4′-CH(CH₃)—O-2′ (referred to as “constrained ethyl” or “cEt”), 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem.,2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379;Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191;; Torsten et al., WO 2004/106356;Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,6409; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the a-L configuration or in the β-D configuration.

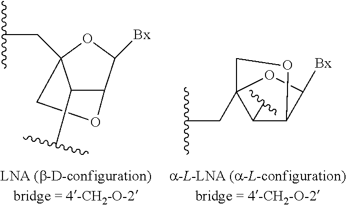

α-L-methyleneoxy (4′-CH₂—O-2′) or a-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

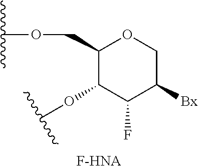

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

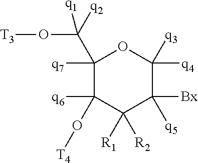

wherein, independently, for each of said modified THP nucleoside:

Bx is a nucleobase moiety;

T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group;
q₁, q₂, q₃, q₄, q₅, q₆ and q₇ are each, independently, H, C₁-C₆ alkyl, substituted C₁-C₆ alkyl, C₂-C₆ alkenyl, substituted C₂- C₆ alkenyl, C₂-C₆ alkynyl, or substituted C₂-C₆ alkynyl; and

each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

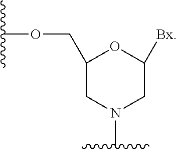

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieites. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

### 2. Certain Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine , 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp) Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S .T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manohara et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

### 3. Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate internucleosides in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate internucleoside. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate internucleoside is present in at least 65% of the molecules in the population. In certain embodiments, the particular configuration of the particular phosphorothioate internucleoside is present in at least 70% of the molecules in the population. In certain embodiments, the particular configuration of the particular phosphorothioate internucleoside is present in at least 80% of the molecules in the population. In certain embodiments, the particular configuration of the particular phosphorothioate internucleoside is present in at least 90% of the molecules in the population. In certain embodiments, the particular configuration of the particular phosphorothioate internucleoside is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

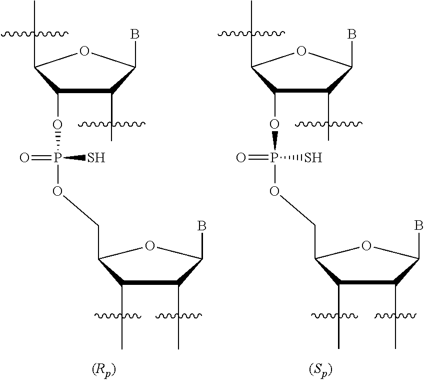

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′—CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

### B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, each nucleoside of each wing of a gapmer is a modified nucleoside. In certain embodiments, at least one nucleoside of each wing of a gapmer is a modified nucleoside. In certain embodiments, at least two nucleosides of each wing of a gapmer are modified nucleosides. In certain embodiments, at least three nucleosides of each wing of a gapmer are modified nucleosides. In certain embodiments, at least four nucleosides of each wing of a gapmer are modified nucleosides.

In certain embodiments, the gap of a gapmer comprises 7-12 nucleosides. In certain embodiments, each nucleoside of the gap of a gapmer is an unmodified 2′-deoxy nucleoside.

In certain embodiments, the gapmer is a deoxy gapmer. In certain embodiments, the nucleosides on the gap side of each wing/gap junction are unmodified 2′-deoxy nucleosides and the nucleosides on the wing sides of each wing/gap junction are modified nucleosides. In certain embodiments, each nucleoside of the gap is an unmodified 2′-deoxy nucleoside. In certain embodiments, each nucleoside of each wing of a gapmer is a modified nucleoside.

Herein, the lengths (number of nucleosides) of the three regions of a gapmer may be provided using the notation [# of nucleosides in the 5′-wing]—[# of nucleosides in the gap]—[# of nucleosides in the 3′-wing]. Thus, a 5-10-5 gapmer consists of 5 linked nucleosides in each wing and 10 linked nucleosides in the gap. Where such nomenclature is followed by a specific modification, that modification is the modification in each sugar moiety of each wing and the gap nucleosides comprise unmodified deoxynucleoside sugars. Thus, a 5-10-5 MOE gapmer consists of 5 linked MOE modified nucleosides in the 5′-wing, 10 linked deoxynucleosides in the gap, and 5 linked MOE nucleosides in the 3′-wing.

In certain embodiments, modified oligonucleotides are 5-10-5 MOE gapmers. In certain embodiments, modified oligonucleotides are 3-10-3 BNA gapmers. In certain embodiments, modified oligonucleotides are 3-10-3 cEt gapmers. In certain embodiments, modified oligonucleotides are 3-10-3 LNA gapmers. In certain embodiments modified oligonucleotides are 5-10-5 OMe/MOE gapmers. In certain embodiments 5-10-5 OMe/MOE gapmers have the motif meeem-10-mmmmm, where m represents a 2′-MOE modification and e represents a 2′-OMe modification.

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, modified oligonucleotides comprise or consist of a region having a fully modified sugar motif, wherein each nucleoside within the fully modified region comprises the same modified sugar moiety (uniformly modified sugar motif). In certain embodiments, the uniformly modified sugar motif is 7 to 20 nucleosides in length. In certain embodiments, each nucleoside of the uniformly modified sugar motif is a 2′-substituted nucleoside, a sugar surrogate, or a bicyclic nucleoside. In certain embodiments, each nucleoside of the uniformly modified sugar motif comprises either a 2′-OCH₂CH₂OCH₃ group or a 2′-OCH₃ group. In certain embodiments, modified oligonucleotides having at least one fully modified sugar motif may also have at least 1, at least 2, at least 3, or at least 4 2′-deoxynucleosides.

In certain embodiments, each nucleoside of the entire modified oligonucleotide comprises a modified sugar moiety (fully modified oligonucleotide). In certain embodiments, a fully modified oligonucleotide comprises different 2′-modifications. In certain embodiments, each nucleoside of a fully modified oligonucleotide is a 2′-substituted nucleoside, a sugar surrogate, or a bicyclic nucleoside. In certain embodiments, each nucleoside of a fully modified oligonucleotide comprises either a 2′-OCH₂CH₂OCH₃ group and at least one 2′-OCH₃ group.

In certain embodiments, each nucleoside of a fully modified oligonucleotide comprises the same 2′-modification (uniformly modified oligonucleotide). In certain embodiments, each nucleoside of a uniformly modified oligonucleotide is a 2′-substituted nucleoside, a sugar surrogate, or a bicyclic nucleoside. In certain embodiments, each nucleoside of a uniformly modified oligonucleotide comprises either a 2′-OCH₂CH₂OCH₃ group or a 2′-OCH₃ group In certain embodiments, modified oligonucleotides comprise at least 12, at last 13, at least 14, at least 15, at least 16, at least 17, at least 18, at least 19, or at least 20 nucleosides comprising a modified sugar moiety. In certain embodiments, each nucleoside of a modified oligonucleotide is a 2′-substituted nucleoside, a sugar surrogate, a bicyclic nucleoside, or a 2′-deoxynucleoside. In certain embodiments, each nucleoside of a modified oligonucleotide comprises a 2′-OCH₂CH₂OCH₃ group, a 2′-H(H) deoxyribosyl sugar moiety, or a cEt modified sugar.

### 2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

### 3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P=o). In certain embodiments, each internucleoside linking group of a modified oligonucleotide is a phosphorothioate internucleoside linkage (P=s). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, the sugar motif of a modified oligonucleotide is a gapmer and the internucleoside linkages within the gap are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain embodiments, the internucleoside linkage motif is sooosssssssssssssss. In certain such embodiments, all of the phosphorothioate internucleosides are stereorandom. In certain embodiments, all of the phosphorothioate internucleosides in the wings are (Sp) phosphorothioates, and the gap comprises at least one Sp, Sp, Rp motif. In certain embodiments, the internucleoside linkage motif is Sp-o-o-o-Sp-Sp-Sp-Rp-Sp-Sp-Rp-Sp-Sp-Sp-Sp-Sp-Sp-Sp-Sp. In certain embodiments, the internucleoside linkage motif is Sp-o-o-o-Sp-Sp-Sp-Rp-Sp-Sp-Sp-Sp-Sp-Sp-Sp-Sp-Sp-Sp-Sp. In certain embodiments, populations of modified oligonucleotides are enriched for modified oligonucleotides comprising such internucleoside linkage motifs.

### C. Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target mRNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X<Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides

### D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

### E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

### F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### II. Certain Olifomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides. Examples of terminal groups include but are not limited to conjugate groups, capping groups, phosphate moieties, protecting groups, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-calbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

### 1. Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to react with to a particular site on a parent compound and the other is selected to react with to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-l-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases. In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate internucleoside. In certain such embodiments, the cleavable moiety is 2′-deoxyadenosine.

### B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphanates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

### III. Oligoomeric Duplexes

In certain embodiments, oligomeric compounds described herein comprise an oligonucleotide, having a nucleobase sequence complementary to that of a target nucleic acid. In certain embodiments, an oligomeric compound is paired with a second oligomeric compound to form an oligomeric duplex. Such oligomeric duplexes comprise a first oligomeric compound having a region complementary to a target nucleic acid and a second oligomeric compound having a region complementary to the first oligomeric compound. In certain embodiments, the first oligomeric compound of an oligomeric duplex comprises or consists of (1) a modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides.

### IV. Antisense Activity

In certain embodiments, oligomeric compounds and oligomeric duplexes comprising modified oligonucleotides provided herein are capable of hybridizing to a target nucleic acid, resulting in at least one antisense activity; such oligomeric compounds and oligomeric duplexes may be referred to as antisense compounds. In certain embodiments, antisense compounds have antisense activity when they modulate, reduce, or increase the amount or activity of a target nucleic acid by 25% or more in the standard cell assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, antisense compounds are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid. In certain embodiments, hybridization of an antisense compound to a target RNA results in exon skipping. In certain embodiments, hybridization of an antisense compound to a target nucleic acid results in an increase or a reduction in the amount or activity of a target nucleic acid. In certain embodiments, hybridization of an antisense compound complementary to a target nucleic acid results in alteration of splicing, leading to the omission of an exon in the mRNA. This alteration of a splice site may be referred to, for example, as splice-switching, or splice skipping, and the alteration of a splice site that leads to the omission of an exon may be referred to as exon skipping, or exon (number) skipping. In certain embodiments, the alteration of a splice site, or exon skipping, may result in elimination of a premature stop codon. In certain embodiments, the alteration of a splice site, or exon skipping, may result in elimination of a frame-shift; in certain embodiments the elimination of a frame-shift may result in elimination of a premature stop codon.

In some embodiments splice switching oligonucleotides alter pre-mRNA splicing; in some embodiments splice switching oligonucleotides comprise or consist of modified nucleic acids; in some embodiments, splice switching oligonucleotides are short oligomers; in some embodiments, splice switching oligonucleotides are stable and are RNase

H resistant; in some embodiments, splice switching oligonucleotides are safe and have low toxicity; in some embodiments, splice switching oligonucleotides are freely taken up by many cells; in some embodiments, splice switching oligonucleotides are FDA approved for treatment of other pediatric diseases.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or animal

### V. Certain Target Nucleic Acids

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target RNA is a mature mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron. In certain embodiments, the target nucleic acid is the RNA transcriptional product of a retrogene. In certain embodiments, the target nucleic acid is a non-coding RNA. In certain such embodiments, the target non-coding RNA is selected from: a long non-coding RNA, a short non-coding RNA, an intronic RNA molecule.

### A. Complementarity/Mismatches to the Target Nucleic Acid

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl -xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and a 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved. In certain embodiments, the mismatch is specifically positioned within an oligonucleotide having a gapmer motif. In certain embodiments, the mismatch is at position 1, 2, 3, 4, 5, 6, 7, or 8 from the 5′-end of the gap region. In certain embodiments, the mismatch is at position 9, 8, 7, 6, 5, 4, 3, 2, 1 from the 3′-end of the gap region. In certain embodiments, the mismatch is at position 1, 2, 3, or 4 from the 5′-end of the wing region. In certain embodiments, the mismatch is at position 4, 3, 2, or 1 from the 3′-end of the wing region.

### B. CLN3

In certain embodiments, oligomeric compounds comprise or consist of an oligonucleotide comprising a region that is complementary to a target nucleic acid, wherein the target nucleic acid is CLN3. In certain embodiments, CLN3 nucleic acid has the sequence set forth in SEQ ID NO: 1 (the complement of GENBANK Accession No: NT_010393.16 truncated from nucleotides 28427600 to 28444620) or SEQ ID NO: 2 (the complement of GENBANK Accession No: NT_039433.8 truncated from nucleotides 44319075 to 44333955).

In certain embodiments, CLN3 nucleic acid has the sequence set forth in SEQ ID NO: 99 (GENBANK accession number NM 001042432.1), SEQ ID NO: 100 (GENBANK accession number NM_000086.2), or SEQ ID NO: 101 (GENBANK accession number NM_001286110.1).

In certain embodiments, contacting a cell with an oligomeric compound complementary to SEQ ID NO: 1 or SEQ ID NO: 2 modulates the expression of CLN3 RNA, in certain embodiments modulates the activity of CLN3 mRNA, and in certain embodiments modulates the activity or amount of CLN3 protein. In certain embodiments, contacting a cell with an oligomeric compound complementary to SEQ ID NO: 99, SEQ ID NO: 100, or SEQ ID NO: 101 modulates the expression of CLN3 RNA, in certain embodiments modulates the activity of CLN3 mRNA, and in certain embodiments modulates the activity or amount of CLN3 protein. In certain embodiments, contacting a cell with an oligomeric compound complementary to SEQ ID NO: 1 or SEQ ID NO: 2 ameliorates one or more symptom or hallmark of a neurodegenerative disease. In certain embodiments, contacting a cell with an oligomeric compound complementary to SEQ ID NO: 99, SEQ ID NO: 100, or SEQ ID NO: 101 ameliorates one or more symptom or hallmark of a neurodegenerative disease. In certain embodiments, the symptom or hallmark is poor motor function, seizures, vision loss, poor cognitive function, psychiatric problems, accumulation of autofluorescent ceroid lipopigment in brain tissue, brain tissue dysfunction, brain tissue cell death, accumulation of mitochondrial ATP synthase subunit C in brain tissue, accumulation of lipofuscin in brain tissue, or astrocyte activation in brain tissue. In certain embodiments, contacting a cell with a modified oligonucleotide complementary to SEQ ID NO: 1 or SEQ ID NO: 2 results in improved motor function, reduced neuropathy, and reduction in number of aggregates. In certain embodiments, contacting a cell with a modified oligonucleotide complementary to SEQ ID NO: 99, SEQ ID NO: 100, or SEQ ID NO: 101 results in improved motor function, reduced neuropathy, and reduction in number of aggregates. In certain embodiments, the oligomeric compound consists of a modified oligonucleotide.

### C. Certain Target Nucleic Acids in Certain Tissues

In certain embodiments, oligomeric compounds comprise or consist of an oligonucleotide comprising a region that is complementary to a target nucleic acid, wherein the target nucleic acid is expressed in a pharmacologically relevant tissue. In certain embodiments, the pharmacologically relevant tissues are the cells and tissues that comprise the central nervous system (CNS). Such tissues include brain tissues, such as, cortex, spinal cord, hippocampus, pons, cerebellum, substantia nigra, red nucleus, medulla, thalamus, and dorsal root ganglia

### VI. Certain Pharmaceutical Compositions

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid. In certain embodiments, the artificial cerebrospinal fluid is pharmaceutical grade.

In certain embodiments, a pharmaceutical composition comprises a modified oligonucleotide and artificial cerebrospinal fluid. In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to an animal, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80TM and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, in ionized (anion) form, or ionized and in association with a cation (salt) form, aqueous solutions of such compounds exist in equilibrium among such forms. For example, a phosphate linkage of an oligonucleotide in aqueous solution exists in equilibrium among free acid, anion, and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or salts thereof” expressly includes all such forms that may be fully or partially protonated/de-protonated/in association with a cation. In certain instances, one or more specific cation is identified.

In certain embodiments, oligomeric compounds disclosed herein are in aqueous solution with sodium. In certain embodiments, oligomeric compounds are in aqueous solution with potassium. In certain embodiments, oligomeric compounds are in artificial CSF. In certain embodiments, oligomeric compounds are in PBS. In certain embodiments, oligomeric compounds are in water. In certain such embodiments, the pH of the solution is adjusted with NaOH and/or HCl to achieve a desired pH.

### Nonlimiting Disclosure and Incorporation by Reference

Each of the literature and patent publications listed herein is incorporated by reference in its entirety. While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein mC indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴_{N,} ¹⁷O or ¹⁸O in place of ¹⁶O and ³⁶S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Effect of Uniformly 2′-MOE Modified Oligonucleotides with Phosphorothioate Internucleoside Linkages on Mouse CLN3 In Vitro-CLN3Δ78/Δ78 cells

Modified oligonucleotides (splice-switching oligonucleotides (SSOs)) complementary to a mouse nucleic acid were designed and tested for their effect on modulating expression of CLN3 RNA in a mouse cell line homozygous for CLN3Δ78 (CLN3Δ78/Δ78). C334E cells, which are homozygous for CLN3Δ78 (CLN3Δ78/Δ78), were generated from the tissue of embryonic day 15 CLN3Δex78 (CLN3Δ78/Δ78) mouse embryos, which contains two copies of mutant CLN3 that lacks exons 7 and 8. In brief, mouse was euthanized, and embryos removed. A transverse section was made above the eyes to isolate the brain tissue and skull was removed. Tissue was minced and incubated in 0.01% trypsin and then cultured in DMEM high glucose+10%FBS+1% pen/strep. Attached and dividing cells were then propagated and expanded. The SSOs were tested for the ability to modulate activity of CLN3 RNA by modulating splicing of CLN3 RNA, inducing skipping of exon 5.

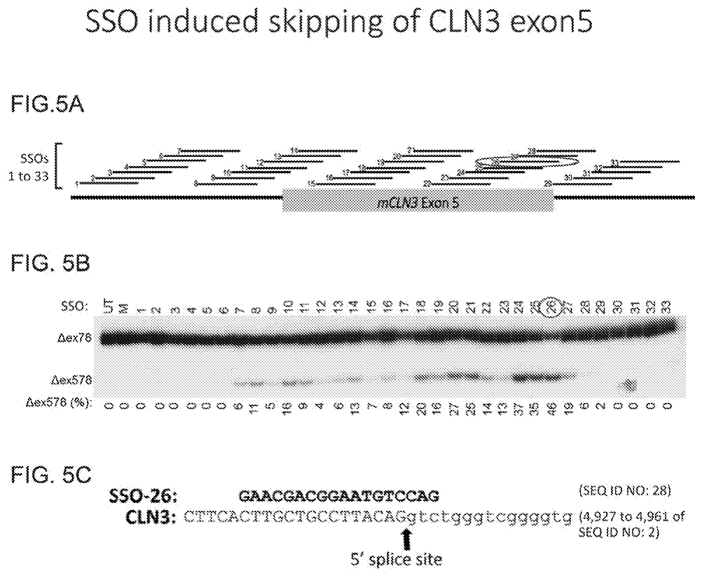

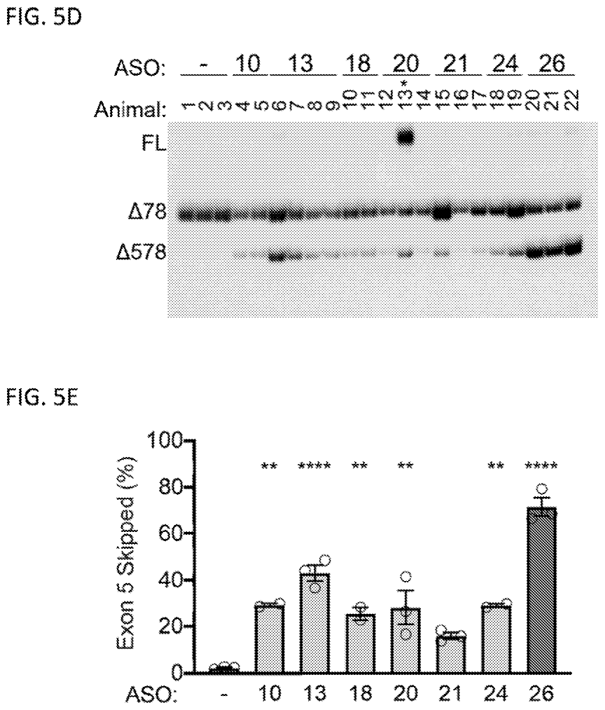

Cells were transfected with 100 nM of the modified oligonucleotides (SSOs) listed in Table 1 using Lipofectamine 2000 (Invitrogen). Untreated control cells received neither modified oligonucleotide nor Lipofectamine, while mock transfected cells received only Lipofectamine. After 48 hours, total RNA was collected from the cells and splicing was analyzed by RT-PCR using primers mCLN3ex4F (5′-CAACTCCATCTCCACAGC-3′) (SEQ ID NO: 54) and mCLN3ex10R (5′-AGAGGTCCCAGCTGGCAC-3′) (SEQ ID NO: 55). PCR products were analyzed on an acrylamide gel and quantitated by phosphorimager analysis (Typhoon 9400, GE Healthcare) (FIG. 5).

The modified oligonucleotides in Table 1 below are uniformly modified oligonucleotides. The oligonucleotides are 18 nucleobases in length. Each nucleoside is a 2′-MOE nucleoside. Each internucleoside linkage is a phosphorothioate internucleoside linkage, and each cytosine residue is a 5-methylcytosine. The nucleobase sequence of each oligonucleotide is listed in the table below. “Start site” indicates the 5′-most nucleoside to which the oligonucleotide is complementary to the mouse CLN3 nucleic acid sequence. “Stop site” indicates the 3′-most nucleoside to which the oligonucleotide is complementary in the mouse CLN nucleic acid sequence.

Each modified oligonucleotide listed in Table 1 below is complementary to SEQ ID NO: 2 (mouse CLN3, the complement of mouse GENBANK accession number NT_039433.8, truncated from nucleotides 44319075 to 44333955). The modified oligonucleotides listed in Table 1 are complementary to exon five and/or the introns flanking exon 5 of the mouse CLN3 pre-mRNA. Modulation of expression of CLN3 for each modified oligonucleotide is listed as exon 5 skipping. The percentage of exon 5 skipping detected in each assay for each modified oligonucleotide is calculated as the percentage of CLN3Δex578 RNA(exon 5 skipped) out of the total CLN3 RNA (i.e., the total of: CLNΔex78 RNA and CLN3Δex578 transcript)(×100).

As shown below, modified oligonucleotides complementary to mouse CLN3 modulated expression of mouse CLN3 RNA. Modified oligonucleotides complementary to mouse CLN3 induced exon 5 skipping in cells expressing disease-associated Δex78 CLN3. “SSO-26” as discussed herein in the context of mouse cells or in vivo mouse treatment, refers to modified oligonucleotide SSO-26 of Table 1.

TABLE 1
Activity of mouse CLN3 with uniformly 2′-MOE
modified oligonucleotides with phosphorothioate
internucleoside linkages in mouse CLN3Δ78/Δ78
cells

| | | | | | | | Exon |
|---|---|---|---|---|---|---|---|
| | | | | | | | 5 |
| | | | | | | | skip- |
| Com- | | | SEQ | SEQ | | | ping |
| pound | | | ID | ID | | | (% |
| Number | Com- | | NO: 2 | NO: 2 | CLN3 | SEQ | of |
| (SSO | pound | Sequence | Start | Stop | Target | ID | total |
| ID) | ID | (5′ to 3′) | Site | Site | Region | NO: | RNA) |
| 1 | 730475 | ACAACCTTCCC | 4807 | 4824 | intron | 3 | 0 |
| | | AACCCAG | | | 4 | | |
| 2 | 730476 | CGGAGACAACC | 4812 | 4829 | intron | 4 | 0 |
| | | TTCCCAA | | | 4 | | |
| 3 | 730477 | CTTCCCGGAGA | 4817 | 4834 | intron | 5 | 0 |
| | | CAACCTT | | | 4 | | |
| 4 | 730478 | TAGACCTTCCC | 4822 | 4839 | intron | 6 | 0 |
| | | GGAGACA | | | 4 | | |
| 5 | 730479 | GAGCCTAGACC | 4827 | 4844 | intron | 7 | 0 |
| | | TTCCCGG | | | 4 | | |
| 6 | 730480 | AGTGAGAGCCT | 4832 | 4849 | intron | 8 | 0 |
| | | AGACCTT | | | 4 | | |
| 7 | 730481 | AACACAGTGAG | 4837 | 4854 | intron | 9 | 6 |
| | | AGCCTAG | | | 4 | | |
| 8 | 730482 | AGGAGAACACA | 4842 | 4859 | intron | 10 | 11 |
| | | GTGAGAG | | | 4 | | |
| 9 | 730483 | GAGACAGGAGA | 4847 | 4864 | intron | 11 | 5 |
| | | ACACAGT | | | 4 | | |
| 10 | 730484 | CGCCTGAGACA | 4852 | 4869 | intron | 12 | 16 |
| | | GGAGAAC | | | 4/ | | |
| | | | | | exon 5 | | |
| 11 | 730485 | AGCACCGCCTG | 4857 | 4874 | intron | 13 | 9 |
| | | AGACAGG | | | 4/exon | | |
| | | | | | 5 | | |
| 12 | 730486 | CTAGGAGCACC | 4862 | 4879 | intron | 14 | 4 |
| | | GCCTGAG | | | 4/exon | | |
| | | | | | 5 | | |
| 13 | 730487 | GTCTGCTAGGA | 4867 | 4884 | exon 5 | 15 | 6 |
| | | GCACCGC | | | | | |
| 14 | 730488 | AGGATGTCTGC | 4872 | 4889 | exon 5 | 16 | 13 |
| | | TAGGAGC | | | | | |
| 15 | 730489 | TGGGAAGGATG | 4877 | 4894 | exon 5 | 17 | 7 |
| | | TCTGCTA | | | | | |
| 16 | 730490 | AAGGGTGGGAA | 4882 | 4899 | exon 5 | 18 | 8 |
| | | GGATGTC | | | | | |
| 17 | 730491 | ATGACAAGGGT | 4887 | 4904 | exon 5 | 19 | 12 |
| | | GGGAAGG | | | | | |
| 18 | 730492 | GTTTGATGACA | 4892 | 4909 | exon 5 | 20 | 20 |
| | | AGGGTGG | | | | | |
| 19 | 730493 | CAGGAGTTTGA | 4897 | 4914 | exon 5 | 21 | 16 |
| | | TGACAAG | | | | | |
| 20 | 730494 | GGCGCCAGGAG | 4902 | 4919 | exon 5 | 22 | 27 |
| | | TTTGATG | | | | | |
| 21 | 730495 | CAAGAGGCGCC | 4907 | 4924 | exon 5 | 23 | 25 |
| | | AGGAGTT | | | | | |
| 22 | 730496 | AAGGCCAAGAG | 4912 | 4929 | exon 5 | 24 | 14 |
| | | GCGCCAG | | | | | |
| 23 | 730497 | AAGTGAAGGCC | 4917 | 4934 | exon 5 | 25 | 13 |
| | | AAGAGGC | | | | | |
| 24 | 730498 | GCAGCAAGTGA | 4922 | 4939 | exon 5 | 26 | 37 |
| | | AGGCCAA | | | | | |
| 25 | 730499 | GTAAGGCAGCA | 4927 | 4944 | exon 5 | 27 | 35 |
| | | AGTGAAG | | | | | |
| 26 | 730500 | GACCTGTAAGG | 4932 | 4949 | exon | 28 | 46 |
| | | CAGCAAG | | | 5/ | | |
| | | | | | intron | | |
| | | | | | 5 | | |
| 27 | 730501 | ACCCAGACCTG | 4937 | 4954 | exon | 29 | 19 |
| | | TAAGGCA | | | 5/ | | |
| | | | | | intron | | |
| | | | | | 5 | | |
| 28 | 730502 | CCCCGACCCAG | 4942 | 4959 | exon | 30 | 6 |
| | | ACCTGTA | | | 5/ | | |
| | | | | | intron | | |
| | | | | | 5 | | |
| 29 | 730503 | TGCCACCCCGA | 4947 | 4964 | intron | 31 | 2 |
| | | CCCAGAC | | | 5 | | |
| 30 | 730504 | CCTCCTGCCAC | 4952 | 4969 | intron | 32 | 0 |
| | | CCCGACC | | | 5 | | |
| 31 | 730505 | GCCTCCCTCCT | 4957 | 4974 | intron | 33 | 0 |
| | | GCCACCC | | | 5 | | |
| 32 | 730506 | ACCCTGCCTCC | 4962 | 4979 | intron | 34 | 0 |
| | | CTCCTGC | | | 5 | | |
| 33 | 730507 | CTCCCACCCTG | 4967 | 4984 | intron | 35 | 0 |
| | | CCTCCCT | | | 5 | | |

### Example 2: Effect of Uniformly 2′-MOE Modified Oligonucleotides with Phosphorothioate Internucleoside Linkages on Mouse CLN2 In Vitro—CLN3+/+ cells

Modified oligonucleotides (splice-switching oligonucleotides (SSOs) complementary to a mouse nucleic acid were designed as described in Example 1, and tested for their effect on CLN3 RNA in a mouse cell line expressing wild-type CLN3 (208e), under the same conditions as Example 1. In Table 2, modulation of expression, or exon 5 skipping, is shown as the percentage of exon 5 skipped (Δex5) CLN3 out of the full-length (FL) RNA plus exon 5 skipped transcripts (×100). N.D. indicates that no data was collected.

As shown below, modified oligonucleotides complementary to mouse CLN3 modulated expression of mouse CLN3 RNA. Modified oligonucleotides complementary to mouse CLN3 induced exon 5 skipping in cells expressing wild type CLN3.

TABLE 2
Activity of mouse CLN3 with uniformly 2′-MOE modified
oligonucleotides with phosphorothioate internucleoside
linkages in mouse wild type CLN3+/+ cells

| Compound Number | Compound | Exon 5 skipping (% |
|---|---|---|
| (SSO ID) | ID | of total mRNA) |
| 1 | 730475 | 0 |
| 2 | 730476 | 0 |
| 3 | 730477 | 0 |
| 4 | 730478 | 0 |
| 5 | 730479 | 5 |
| 6 | 730480 | 0 |
| 7 | 730481 | 10 |
| 8 | 730482 | 5 |
| 9 | 730483 | 4 |
| 10 | 730484 | 32 |
| 11 | 730485 | N.D. |
| 12 | 730486 | 31 |
| 13 | 730487 | 37 |
| 14 | 730488 | 24 |
| 15 | 730489 | 12 |
| 16 | 730490 | 12 |
| 17 | 730491 | 31 |
| 18 | 730492 | 56 |
| 19 | 730493 | 27 |
| 20 | 730494 | 42 |
| 21 | 730495 | 41 |
| 22 | 730496 | 29 |
| 23 | 730497 | 36 |
| 24 | 730498 | 40 |
| 25 | 730499 | 37 |
| 26 | 730500 | 69 |
| 27 | 730501 | 25 |
| 28 | 730502 | 0 |
| 29 | 730503 | 0 |
| 30 | 730504 | 0 |
| 31 | 730505 | 0 |
| 32 | 730506 | 0 |
| 33 | 730507 | 0 |

### Example 3: Effect of Uniformly 2′-MOE Modified Oligonucleotides with Phosphorothioate Internucleoside Linkages on Mouse CLN2 In Vitro—CLN3Δ78/Δ78 Cells

Additional modified oligonucleotides (splice-switching oligonucleotides (SSOs)) complementary to a mouse nucleic acid were designed and tested for their effect on CLN3 RNA in a mouse cell line homozygous for CLN3Δ78 (CLN3Δ78/Δ78).

C334E cells were transfected with 200 nM of the modified oligonucleotides listed in Table 3, using the methods of Example 1.

The modified oligonucleotides of Table 3 below are uniformly modified oligonucleotides. The oligonucleotides are 18 nucleobases in length. Each nucleoside has a 2′-MOE group. Each internucleoside linkage is a phosphorothioate internucleoside linkage, and each cytosine residue is a 5-methylcytosine. The nucleobase sequence of each oligonucleotide is listed in the table below. “Start site” indicates the 5′-most nucleoside to which the oligonucleotide is complementary to the mouse CLN3 nucleic acid sequence. “Stop site” indicates the 3′-most nucleoside to which the oligonucleotide is complementary in the mouse CLN nucleic acid sequence.

Each modified oligonucleotide listed in Table 3 below is complementary to SEQ ID NO: 2 (mouse CLN3, the complement of mouse GENBANK accession number NT_039433.8, truncated from nucleotides 44319075 to 44333955). The modified oligonucleotides listed in Table 3 are complementary to exon five and/or the introns flanking exon 5 of the mouse CLN3 pre-mRNA. As shown below, modified oligonucleotides complementary to mouse CLN3 modulated expression of mouse CLN3 RNA. Modified oligonucleotides complementary to mouse CLN3 induced exon 5 skipping in cells expressing disease-associated Δex78 CLN3.

In Table 3, modulation of expression, or exon 5 skipping, is shown as the percentage of exon 5 skipped (Δex5) CLN3 out of the full-length (FL) RNA plus exon 5 skipped transcripts (×100).

TABLE 3
Activity of splice-switching oligonucleotides
complementary to mouse CLN3 RNA in CLN3Δ78/Δ78
mouse cells

| | | Start | Stop | | | |
|---|---|---|---|---|---|---|
| | | Site | Site | | | |
| | | on | on | | | |
| | | SEQ | SEQ | | SSO | Exon |
| Com- | | ID | ID | CLN3 | SEQ | skip- |
| pound | SSO Sequence | NO: | NO: | Target | ID | ping |
| ID | (5′ to 3′) | 2 | 2 | Region | NO: | % |
| 857391 | GCTAGGAGCACCGCCTGA | 4863 | 4880 | intron | 36 | 10 |
| | | | | 4/exon | | |
| | | | | 5 | | |
| 857392 | TGCTAGGAGCACCGCCTG | 4864 | 4881 | intron | 37 | 26 |
| | | | | 4/exon | | |
| | | | | 5 | | |
| 857393 | CTGCTAGGAGCACCGCCT | 4865 | 4882 | intron | 38 | 30 |
| | | | | 4/exon | | |
| | | | | 5 | | |
| 857394 | TCTGCTAGGAGCACCGCC | 4866 | 4883 | intron | 39 | 25 |
| | | | | 4/exon | | |
| | | | | 5 | | |
| 857395 | TGTCTGCTAGGAGCACCG | 4868 | 4885 | exon 5 | 40 | 33 |
| 857396 | ATGTCTGCTAGGAGCACC | 4869 | 4886 | exon 5 | 41 | 34 |
| 857397 | GATGTCTGCTAGGAGCAC | 4870 | 4887 | exon 5 | 42 | 31 |
| 857398 | GGATGTCTGCTAGGAGCA | 4871 | 4888 | exon 5 | 43 | 36 |
| 857399 | TGTAAGGCAGCAAGTGAA | 4928 | 4945 | exon 5 | 44 | 60 |
| 857400 | CTGTAAGGCAGCAAGTGA | 4929 | 4946 | exon 5 | 45 | 75 |
| 857401 | CCTGTAAGGCAGCAAGTG | 4930 | 4947 | exon | 46 | 78 |
| | | | | 5/ | | |
| | | | | intron | | |
| | | | | 5 | | |
| 857402 | ACCTGTAAGGCAGCAAGT | 4931 | 4948 | exon | 47 | 71 |
| | | | | 5/ | | |
| | | | | intron | | |
| | | | | 5 | | |
| 857403 | AGACCTGTAAGGCAGCAA | 4933 | 4950 | exon | 48 | 73 |
| | | | | 5/ | | |
| | | | | intron | | |
| | | | | 5 | | |
| 857404 | CAGACCTGTAAGGCAGCA | 4934 | 4951 | exon | 49 | 64 |
| | | | | 5/ | | |
| | | | | intron | | |
| | | | | 5 | | |
| 857405 | CCAGACCTGTAAGGCAGC | 4935 | 4952 | exon | 50 | 51 |
| | | | | 5/ | | |
| | | | | intron | | |
| | | | | 5 | | |
| 857406 | CCCAGACCTGTAAGGCAG | 4936 | 4953 | exon | 51 | 53 |
| | | | | 5/ | | |
| | | | | intron | | |
| | | | | 5 | | |

### Example 4: Distribution of Modified Oligonucleotides in the Mouse CNS

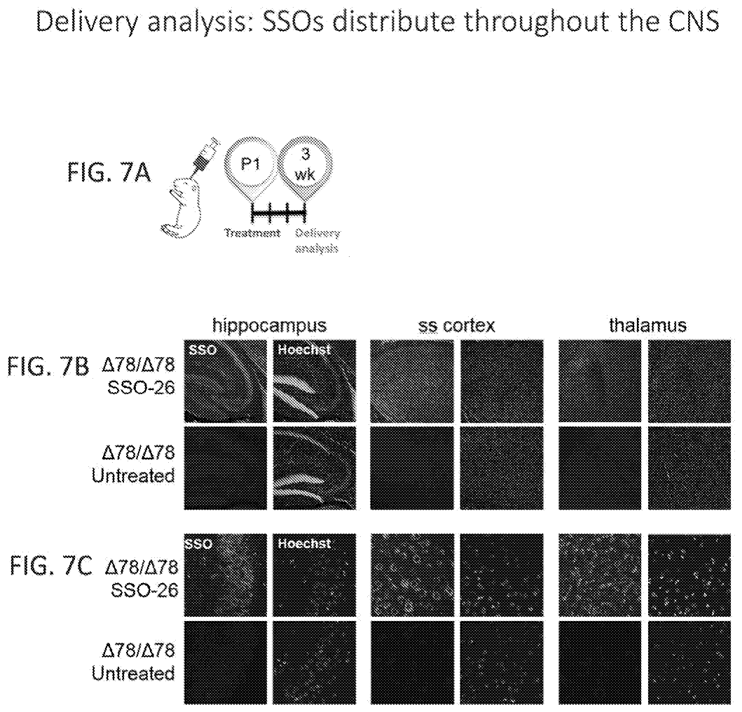

FIG. 7 shows that modified oligonucleotides (SSOs) are widely distributed in the CNS. Modified oligonucleotide SSO-26 (aka SSO 26, SSO-26, Compound ID 730500, SEQ ID NO: 28) was administered via neonatal ICV injection to CLN3Δ78/Δ78 mice, and modified oligonucleotide delivery was analyzed at 3 weeks post-injection.

Immunofluorescent staining of the modified oligonucleotide is shown in the left four panels of each set of images, while Hoechst (nuclear) staining is shown on the right. FIG. 7B shows pairs of images in the hippocampus, the somatosensory cortex, and the thalamus at 10×magnification for CLN3Δ78/Δ78 mice treated with SSO-26 (top) and untreated CLN3Δ78/Δ78 mice (bottom). FIG. 7C shows images of the same tissues at 60×magnification. The treated animals display oligonucleotide staining in the hippocampus, somatosensory cortex, and thalamus. No signal is detected in the oligonucleotide panels for untreated animals, and similar levels of staining are seen for Hoechst staining, indicating that the tissues imaged contain approximately the same number of cells.

### Example 5: Modified Oligonucleotides for Inducing Mouse CLN3 Exon 5 Skipping in a Mouse Model of Batten Disease

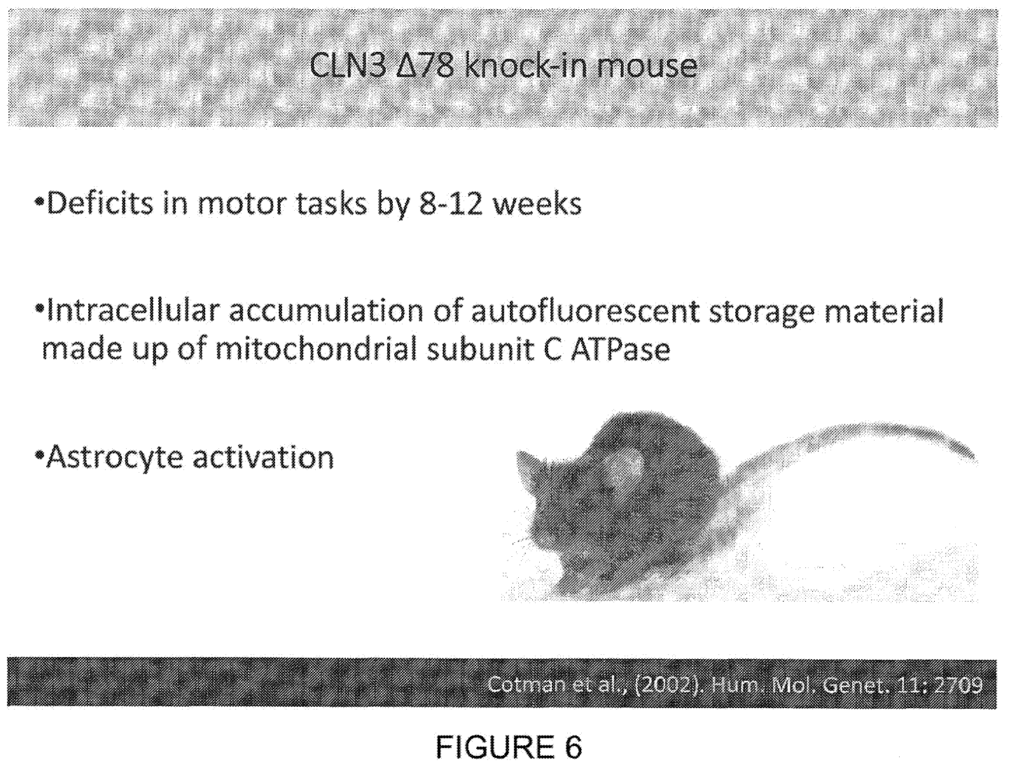

Modified oligonucleotides provided in Tables 1-3 above were tested in an in vivo model of Batten Disease (FIG. 6). The mouse model has a genomic DNA deletion of a 1kb region in the mouse CLN3 gene corresponding to the CLN3Δex78 deletion that underlies most cases of Batten Disease (Cotman, et al., Hum. Mol. Genetics, 11(22):2709-2721, 2002). These homozygous CLN3Δ78/Δ78 mice exhibit symptoms of Batten Disease, including deficits in motor tasks by 8-12 weeks of age.

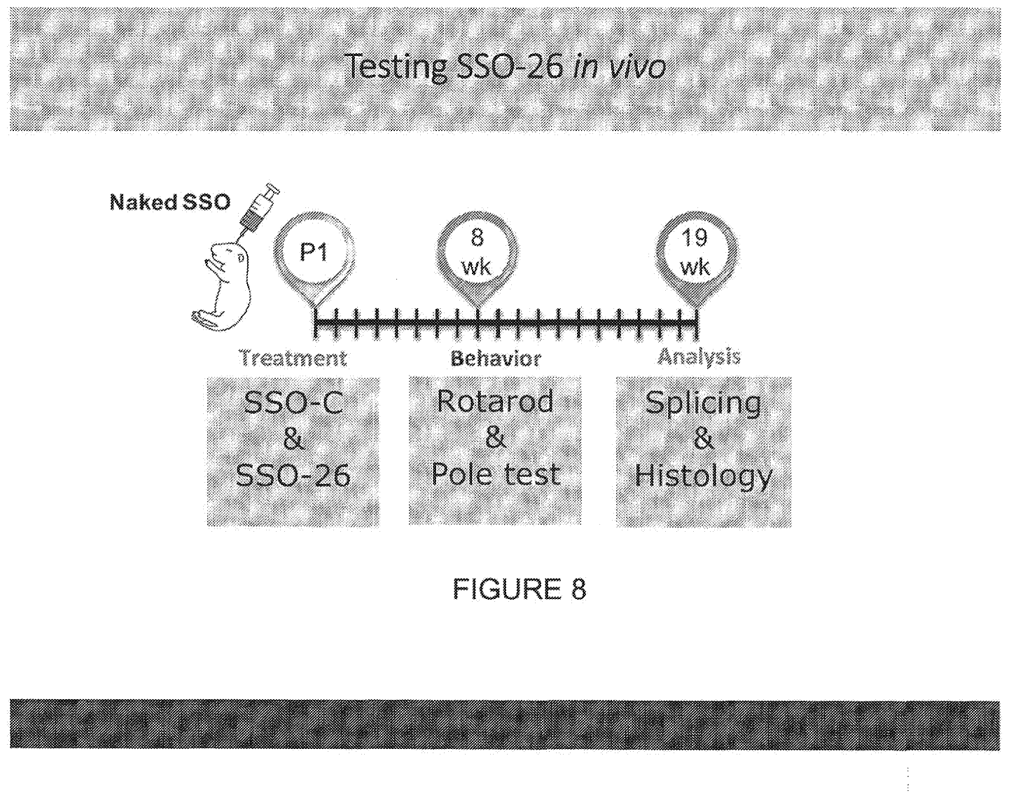

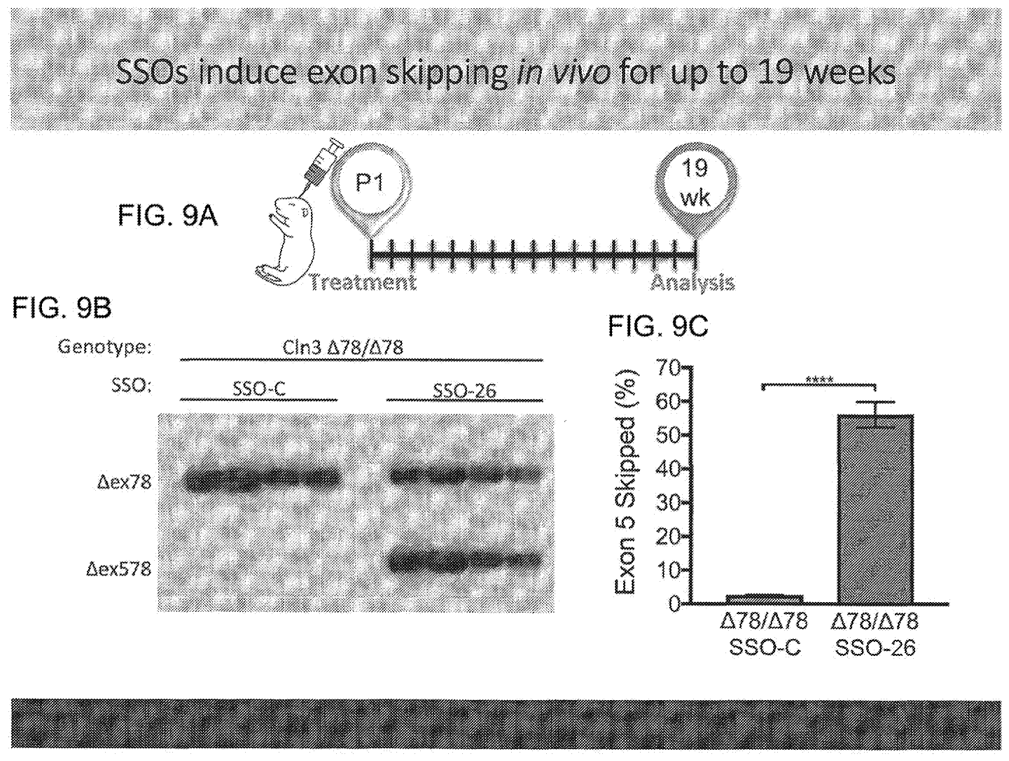

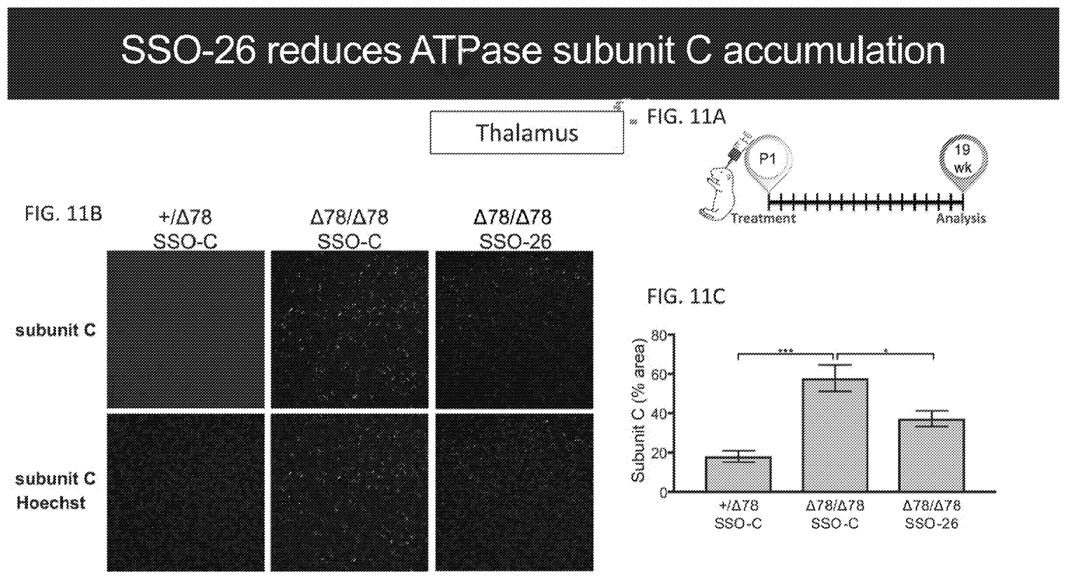

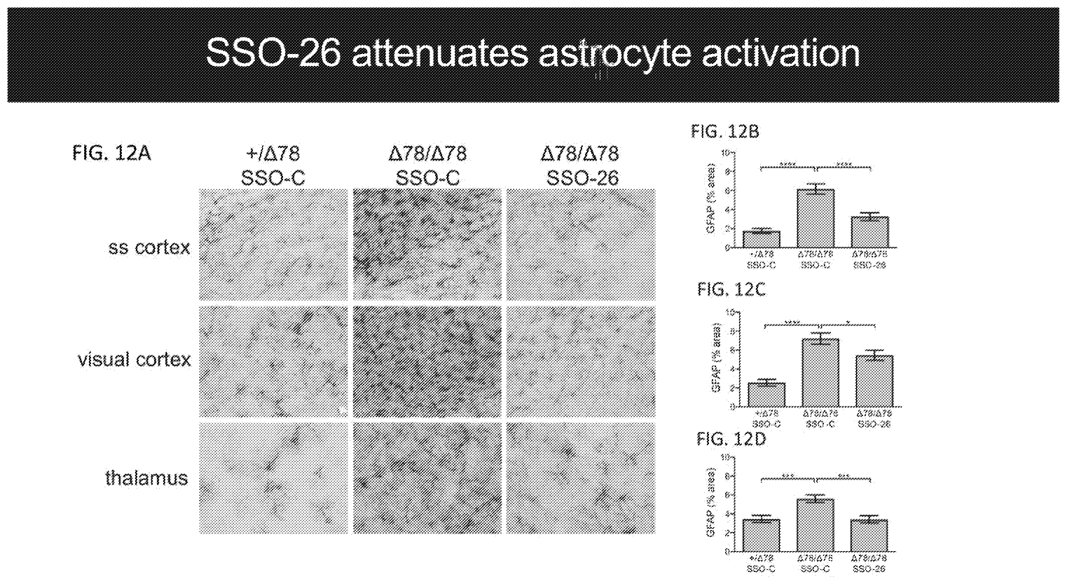

Homozygous CLN3Δ78/Δ78 mice were injected with 500 μg mouse modified oligonucleotide SSO-26 or a control modified oligonucleotide by ICV injection on post-natal day 1, and splicing was analyzed at 3 weeks, 19 weeks, and 26 weeks. The control modified oligonucleotide (SSO-C) is not 100% complementary to any known mouse genes. It has a sequence of TTAGTTTAATCACGCTCG (SEQ ID NO: 97; Compound ID 439272) where each nucleoside is a 2′-MOE nucleoside, each internucleoside linkage is a phosphorothioate internucleoside linkage, and each cytosine nucleobase is a 5-methylcytosine. N.D. indicates that data was not collected for that condition. The timeline of the experiment to 19 weeks is provided in the schematic of FIG. 8. The results, shown in FIGS. 9 and 11, and in Table 4 below, show that a single dose of a modified oligonucleotide can modulate expression of mouse CLN3 by modulating the splicing of mouse CLN3 in vivo for up to 26 weeks.

TABLE 4
Effects of a modified oligonucleotides (splice-switching oligonucleotide)
in a mouse model of Batten Disease in vivo

| | % Exon 5 skipped CLN3 | | |
|---|---|---|---|
| | 3 weeks | 19 weeks | 6 months |
| Treatment | (N = 5) | (N = 8) | (N = 4). |
| Control modified | N.D. | 2.6 | 3.3 |
| oligonucleotide SSO-C | | | |
| Mouse modified | 56 | 56 | 54 |
| oligonucleotide SSO-26 | | | |
| (730500) | | | |

### Example 6: Modified Oligonucleotides for Inducing Human CLN3 Exon 5 Skipping in a Mouse Model of Batten Disease

Modified oligonucleotides described above were tested in an in vivo model of Batten Disease.

Homozygous CLN3Δ78/Δ78 mice were injected with 500 μg modified oligonucleotide by ICV injection at 8 weeks of age, and splicing of CLN3 was analyzed two weeks later. The results show that several splice-switching oligonucleotides complementary to exon 5 or the flanking introns of exon 5 can induce splice-switching of CLN3 in vivo.

TABLE 5
Effects of Splice-switching oligonucleotide
in a mouse model of Batten Disease

| Treatment Mouse SSO # | |
|---|---|
| (Compound ID) | % Exon 5 skipped CLN3 |
| PBS | 9.3 |
| Mouse SSO #10 (730484) | 35.0 |
| Mouse SSO #13 (730487) | 49.5 |
| Mouse SSO #18 (730492) | 30.9 |
| Mouse SSO #20 (730494) | 33.5 |
| Mouse SSO #21 (730495) | 19.9 |
| Mouse SSO #24 (730498) | 34.8 |
| Mouse SSO-26 (730500) | 76.4 |

### Example 7: Modified Oligonucleotides Improve Symptoms in an In Vivo Mouse Model of Batten Disease

Homozygous CLN3Δ78/Δ78 mice, discussed in FIG. 6 and in Example 5 above, were injected with 25 μg mouse modified oligonucleotide SSO-26 or a control modified oligonucleotide by ICV injection on post-natal day 1. Behavior of the treated and the control mice was assessed 8 weeks later. The behavior of heterozygous mice (CLN3+/Δ78) was also tested with the control oligonucleotide.

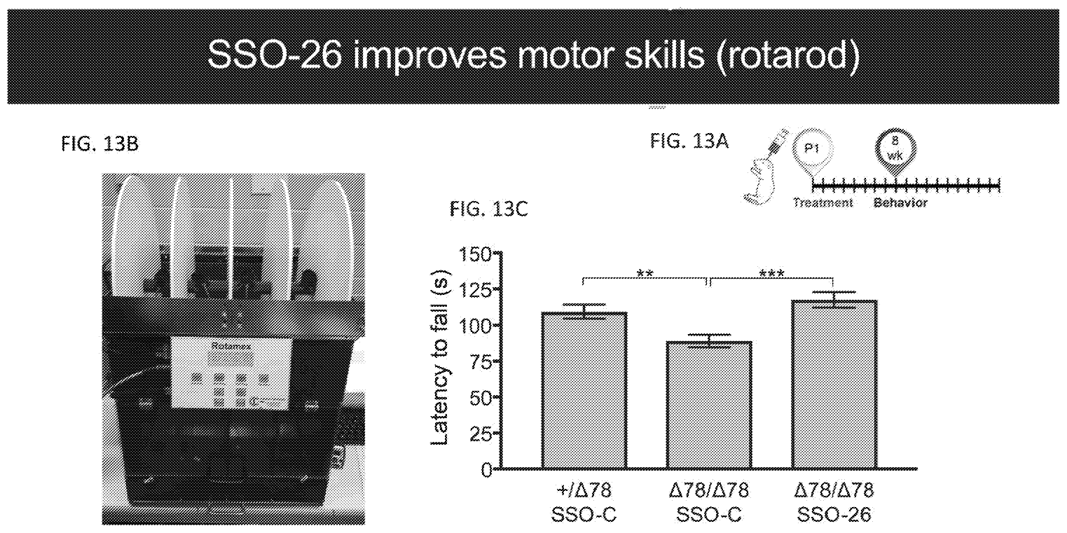

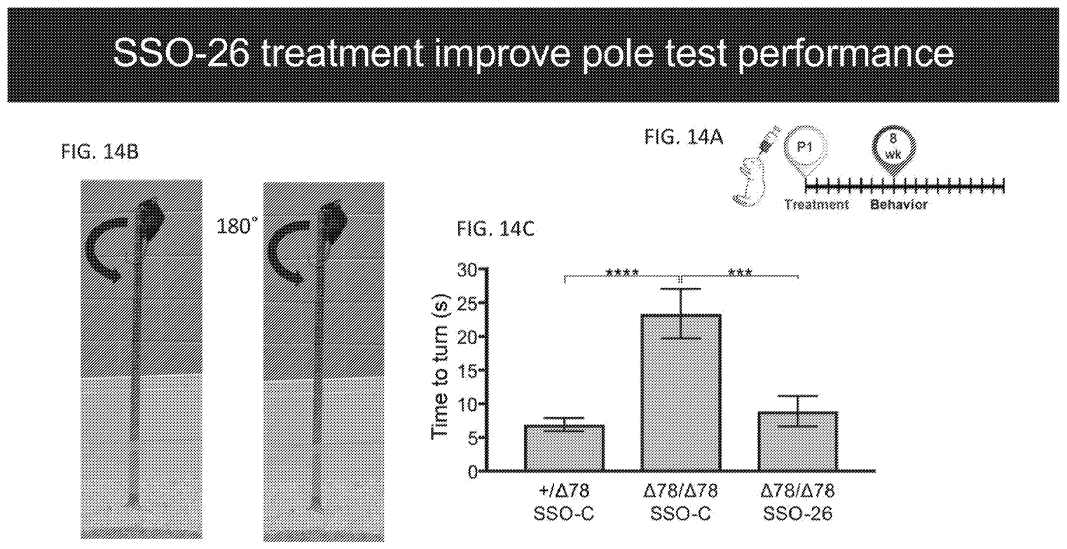

Mice were assessed in an accelerating rotarod test, where a rod accelerated over time, and the latency to fall was recorded. Mice were also assessed in the vertical pole test, where mice climb to the top of a pole and the time to turn around is recorded. These motor function tests are described in detail in Karl, et al., Exper. And Tox. Pathology, 55(1):69-83, 2003. Results are shown in FIGS. 13 and 14 and are quantified in Table 6 below. Treatment of a CLN3Δ78/Δ78 mouse with mouse modified oligonucleotide SSO-26 restored motor symptoms to those of the heterozygous CLN3+/Δ78 mouse.

TABLE 6
Behavioral effects of modified oligonucleotide Splice-switching
oligonucleotide in a mouse model of Batten Disease

| Mouse CLN3 | | Rotarod | Vertical pole test |
|---|---|---|---|
| Genotype | Treatment | latency to fall (s) | time to turn (s) |
| +/Δ78 | control | 109 | 6.9 |
| Δ78/Δ78 | control | 89 | 23.3 |
| Δ78/Δ78 | SSO-26/730500 | 117 | 6.9 |

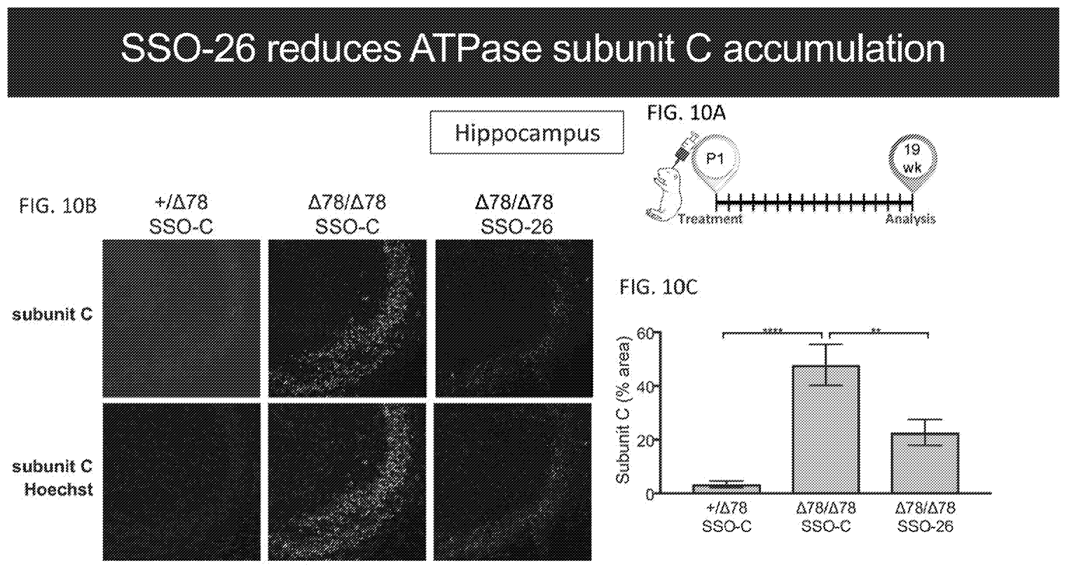

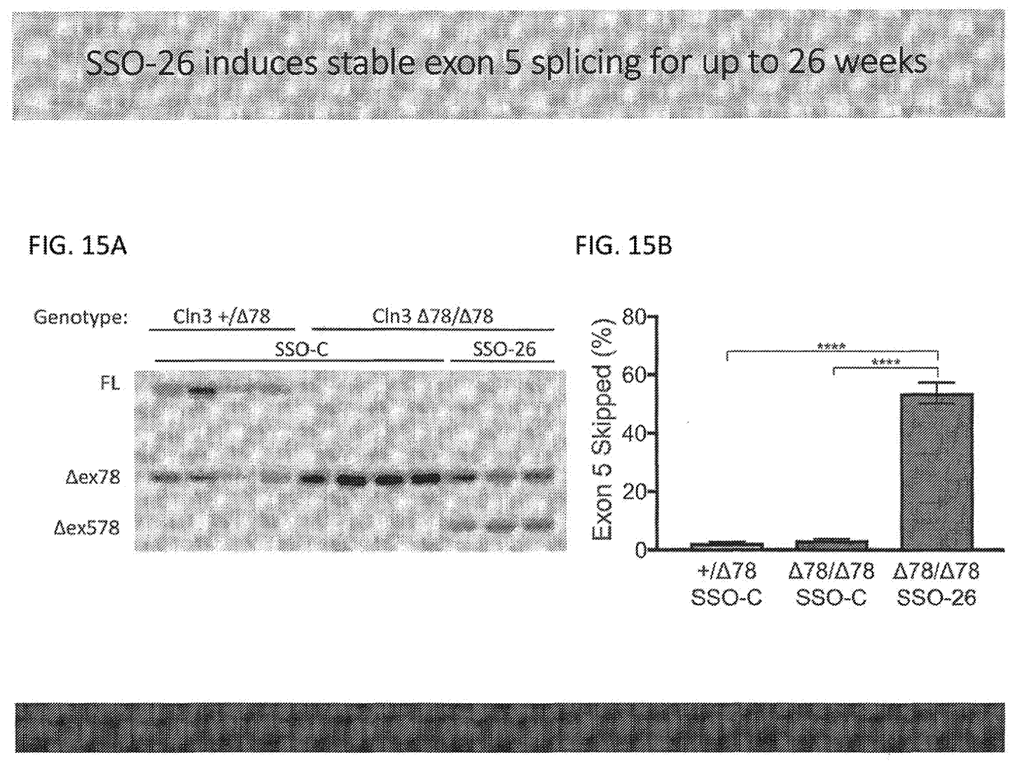

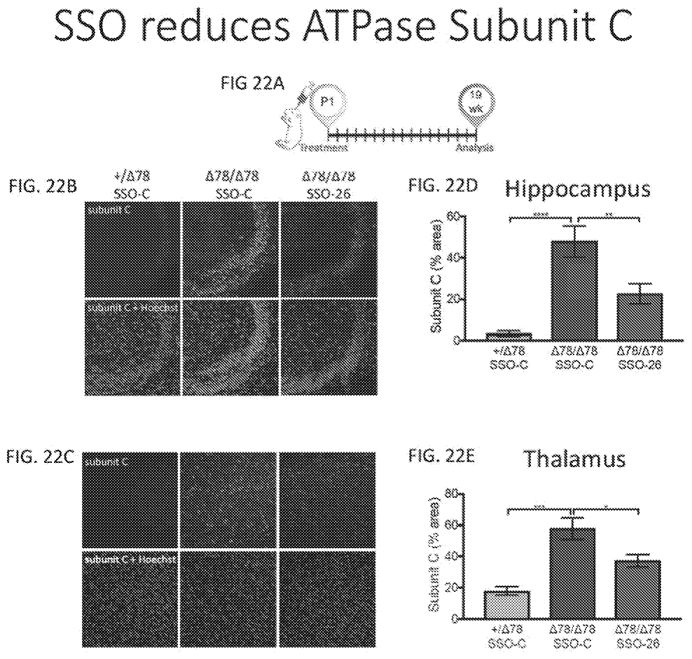

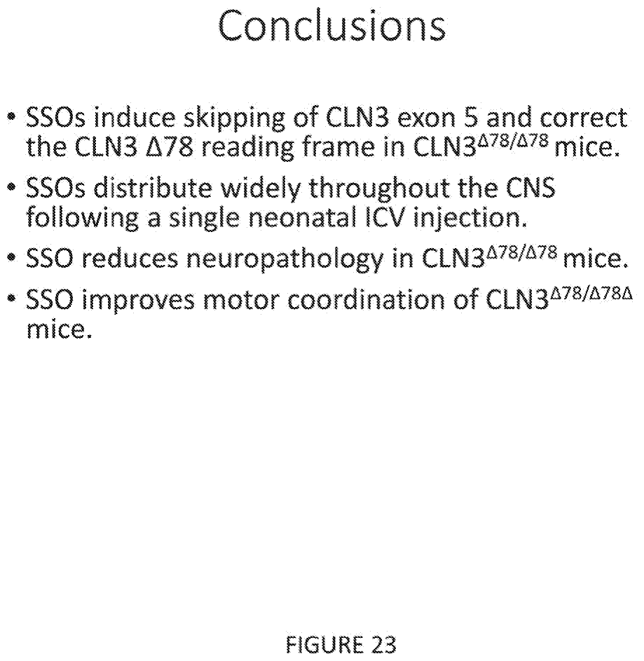

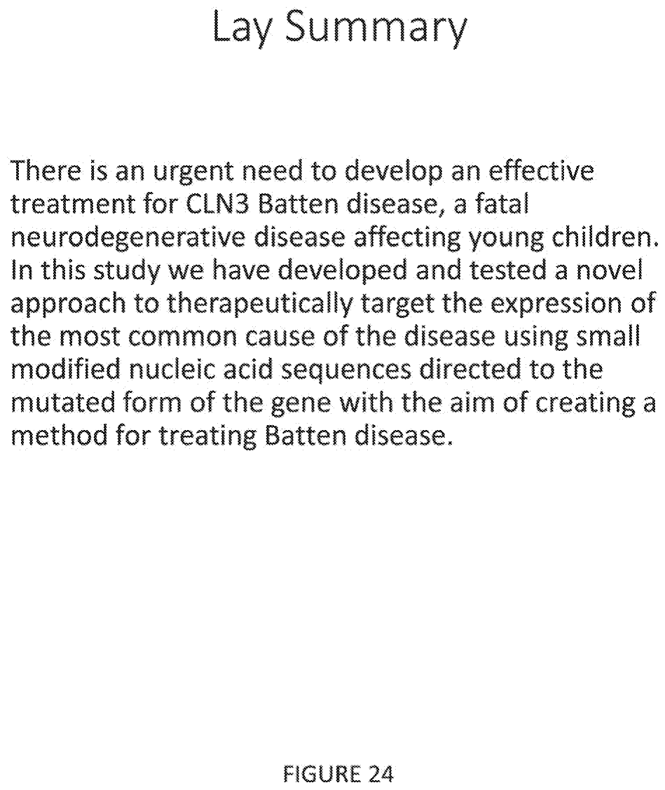

After 19 weeks, mice were sacrificed and tissues were analyzed by histology. Tissue from the hippocampus and thalamus was stained for ATPase subunit C(1:100; Abcam Ab181243) and Hoechst nuclear stain (see FIG. 15). Images were analyzed with Zeiss LSM510 confocal microscope (Carl Zeiss, Oberkochen, Germany) using a 20× objective. Images were collected as vertical z-stacks with 0.74 μm interval and were projected as maximum intensity projections using the Zen software. The total area that stained positive for ATPase subunit C was compared to the total image area. Treatment of CLN3Δ78/Δ78 mice with a splice-switching oligonucleotide leads to reduced ATPase subunit C accumulation in brain tissues (FIGS. 10, 11, and 22).

TABLE 7
Effect of a modified oligonucleotide on ATPase subunit C accumulation
in brain tissues of CLN3Δ78/Δ78 mice

| | | Hippocampus | Thalamus |
|---|---|---|---|
| | | ATPase | ATPase |
| Mouse CLN3 | | subunit C | subunit C |
| Genotype | Treatment | (% area) | (% area) |
| +/Δ78 | control | 3.4 | 18.0 |
| Δ78/Δ78 | control | 47.8 | 57.9 |
| Δ78/Δ78 | SSO-26/730500 | 22.7 | 37.2 |

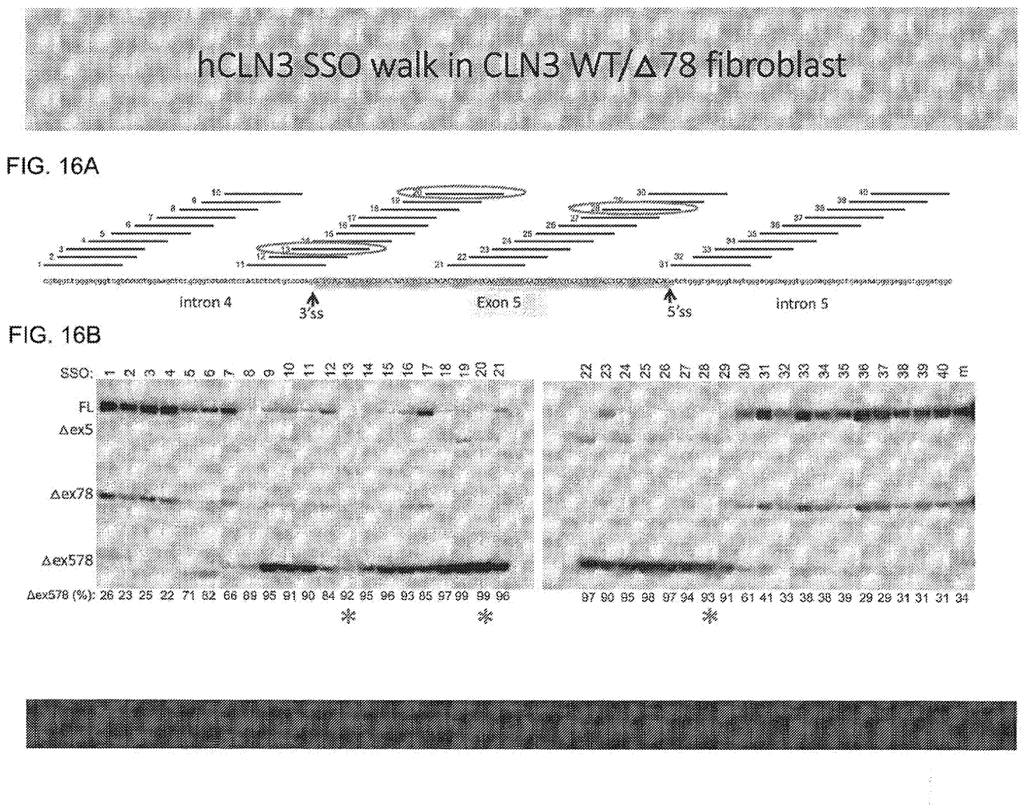

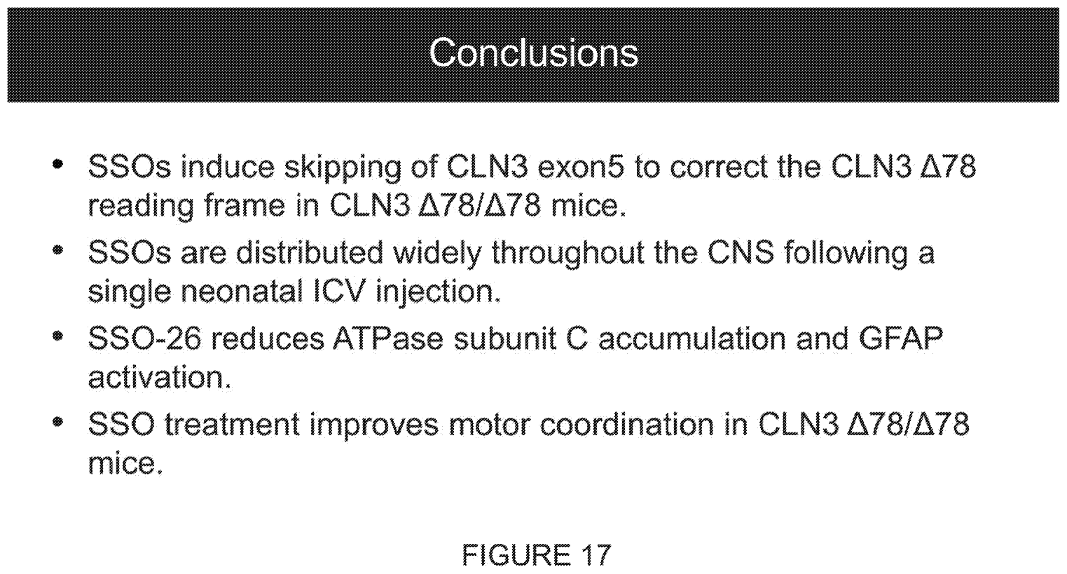

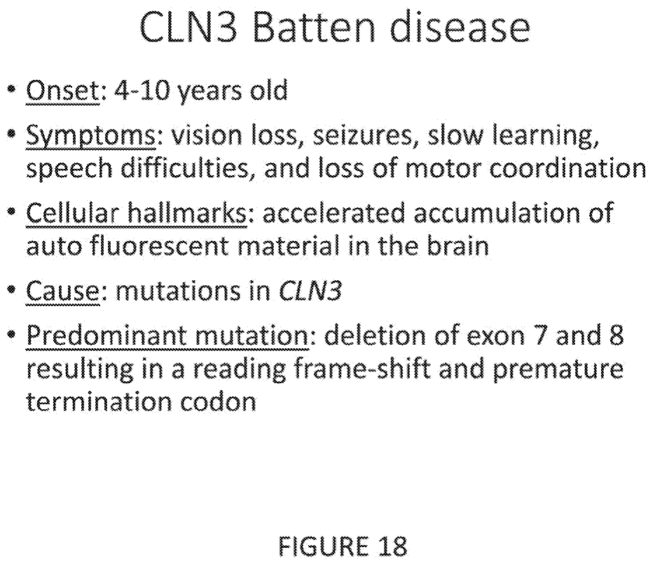

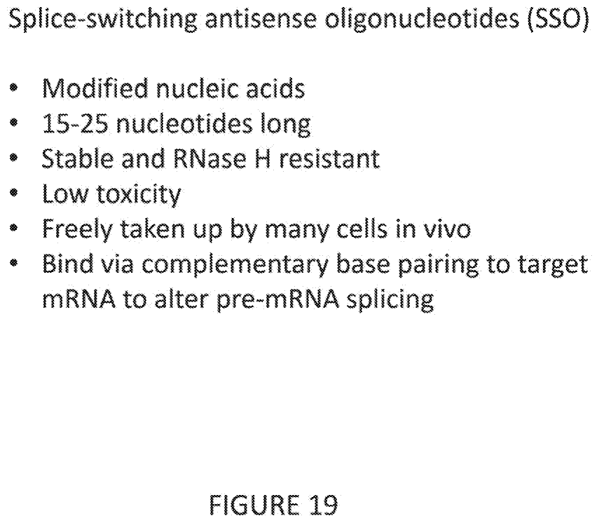

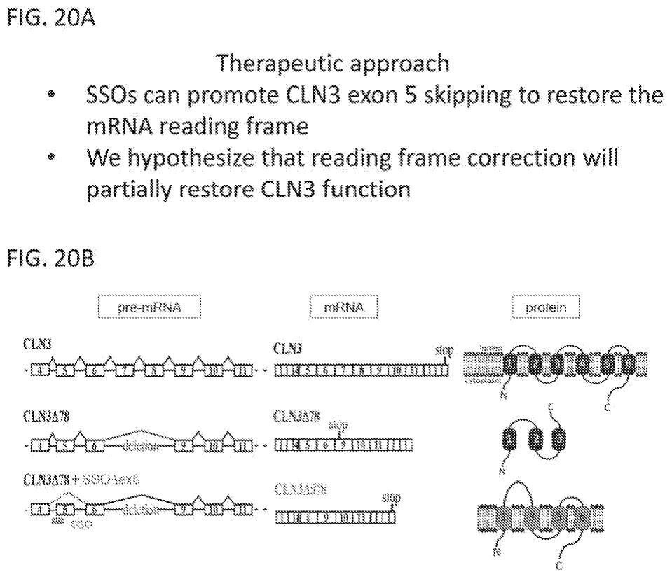

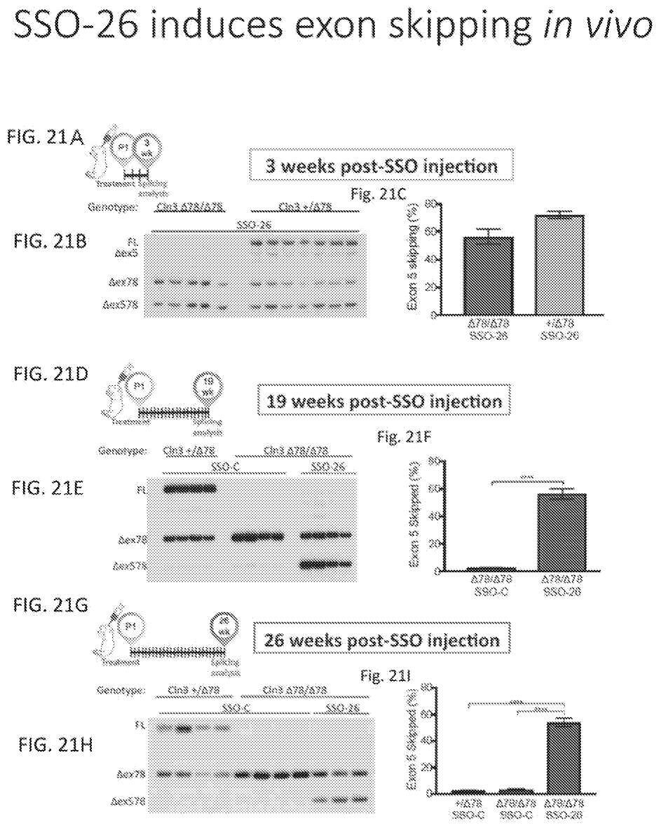

Brain tissues including the somatosensory cortex, visual cortex, and thalamus were also analyzed for astrocyte activation by staining for GFAP using anti-GFAP (Dako, Z0334; 1:250). Tissues were then washed 3 times and incubated in anti-rabbit biotinylated secondary antibody (Vector Labs, BA-9400; 1:2,000) diluted in TBS-T +10% goat serum for 2 hours. Tissues were washed and incubated in an ABC amplification kit (Vector Labs) for 2 hours. Tissues were washed and incubated in 0.05% DAB solution until suitable reaction occurred. Tissues were then washed 3 times, mounted, and immersed in xylene for 10 minutes. Next, the slices were coverslipped using DPX mounting media. For DAB staining, slides were scanned on a Leica DM6000B slide-scanning microscope at 20× . . . Images were then extracted from respective regions at 2,400×2,400 pixel dimension for image threshold analysis using ImageJ (FIG. 16). The data are presented in the table below. Treatment of CLN3Δ78/Δ78 mice with a splice-switching oligonucleotide leads to reduced astrocyte activation in brain tissues.

TABLE 8
Effect of a modified oligonucleotide on astrocyte activation
in brain tissues of CLN3Δ78/Δ78 mice

| | | ss cortex | visual | thalamus |
|---|---|---|---|---|
| Mouse CLN3 | | GFAP | cortex GFAP | GFAP |
| Genotype | Treatment | (% area) | (% area) | (% area) |
| +/Δ78 | control | 1.7 | 2.5 | 3.4 |
| Δ78/Δ78 | control | 6.2 | 7.2 | 5.6 |
| Δ78/Δ78 | SSO-26/730500 | 3.3 | 5.4 | 3.4 |

### Example 8: Modified Oligonucleotides Improve Survival in Severe Mouse Model of Batten Disease

A severe mouse model of Batten Disease was developed by crossing the CLN3Δ78/Δ78 mice with mice expressing the hAPP695 cDNA, which encodes a version of human amyloid precursor protein that is prone to aggregation. Additionally, the hAPP695 cDNA with V717F, K670N and M671L was introduced into mice with a wild-type (CLN3+/+) and heterozygous (CLN3+/Δ78) background. CLN3Δ78/Δ78 mice expressing hAPP695 cDNA experience an increased accumulation of hAPP in lysosomes compared to CLN3^{+/+} mice expressing hAPP695cDNA, resulting in increased risk of premature death.

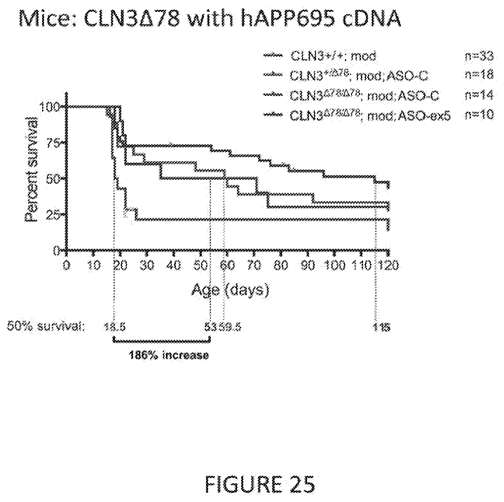

Survival of the CLN3Δ78/Δ78/hAPP695 mice with a control oligonucleotide and with mouse modified oligonucleotide SSO-26 was tracked after administration of 25 μg of modified oligonucleotide at post-natal day 1 via ICV injection. Survival of untreated CLN^{+/+}hAPP695 mice and CLN3+/Δ78/hAPP695 mice treated with a control oligonucleotide on post-natal day 1 via ICV injection was also tracked. The survival curves are presented in FIG. 25 and a summary is presented in Table 9 below. Treatment of Δ78/Δ78 CLN3Δ78/Δ78/hAPP mice with mouse modified oligonucleotide SSO-26 increased survival to the levels of survival seen in heterozygous CLN3+/Δ78/hAPP695 mice. Median survival is the time for 50% of the mice in a given treatment group to experience death and is reported in the table below. Treatment of CLN3Δ78/Δ78 mice with modified oligonucleotide complementary to CLN3 nucleic acid extended median survival, as compared to CLN3Δ78/Δ78 mice that were not treated with the modified oligonucleotide.

TABLE 9
Extension of survival in a severe mouse model
of Batten Disease by a modified oligonucleotide

| | | | Number of mice | Median |
|---|---|---|---|---|
| CLN3 | hAPP | | in treatment | Survival |
| genotype | genotype | Treatment | group | (days) |
| +/+ | hAPP695 | none | 33 | 115 |
| +/Δ78 | hAPP695 | control | 18 | 59.5 |
| Δ78/Δ78 | hAPP695 | control | 14 | 18.5 |
| Δ78/Δ78 | hAPP695 | Mouse modified | 10 | 53 |
| | | oligonucleotide | | |
| | | SSO-26 (730500) | | |

### Example 9: Effect of Uniformly 2′-MOE Modified Oligonucleotides with Phosphorothioate Internucleoside Linkages on Human CLN3 In Vitro —Human CLN3+/Δ78 Cells

Modified oligonucleotides (splice-switching oligonucleotides (SSOs)) complementary to a human nucleic acid were designed and tested for their effect on CLN3 RNA in a human fibroblast cell line heterozygous for CLNΔex78 (CLN3+/Δ78). Cells were transfected with 100 nM of the modified oligonucleotides (SSOs) listed in Table 10 using Lipofectamine 2000 (Invitrogen). Untreated control cells received neither modified oligonucleotide nor Lipofectamine, while mock transfected cells received only Lipofectamine. After 48 hours, total RNA was collected from the cells and RT-PCR was used to identify CLN3Δex78 and CLN3Δex578 transcripts using primers hCLN3ex4F (5′-GCAACTCTGTCTCTACGGC-3′) (SEQ ID NO: 52) and hCLN3ex9R (5′-GCCTCAGGAGATGTGAGC-3′) (SEQ ID NO: 56). The PCR products were analyzed by acrylamide gel electrophoresis and quantitated by phosphorimager analysis (Typhoon 9400, GE Healthcare) and the results are shown in Table 10 below.

The modified oligonucleotides in Table 10 below are uniformly modified oligonucleotides. The oligonucleotides are 18 nucleobases in length. Each nucleoside is a 2′-MOE nucleoside. Each internucleoside linkage is a phosphorothioate internucleoside linkage, and each cytosine residue is a 5-methylcytosine. The nucleobase sequence of each oligonucleotide is listed in the table below. “Start site” indicates the 5′-most nucleoside to which the oligonucleotide is complementary to the human CLN3 nucleic acid sequence. “Stop site” indicates the 3′-most nucleoside to which the oligonucleotide is complementary in the human CLN nucleic acid sequence.

Each modified oligonucleotide of Table 10 is complementary to SEQ ID NO: 1 (human CLN3 nucleic acid, the complement of GENBANK accession number NT_010393.16 truncated from nucleotides 28427600 to 28444620).The modified oligonucleotides listed in Table 10 are complementary to exon five and/or the introns flanking exon 5 of the human CLN3 pre-mRNA. Modulation of expression of CLN3 RNA for each modified oligonucleotide is listed as exon 5 skipping. The percentage of exon 5 skipping detected in each assay for each modified oligonucleotide is calculated as the percentage of CLNΔ578 RNA(exon 5 skipped) out of total CLN3 RNA (i.e., [Δ578/(0578+Δ78)]×100]).

As shown below, modified oligonucleotides complementary to human CLN3 modulated expression of human CLN3 RNA. Modified oligonucleotides complementary to human CLN3 induced exon 5 skipping in cells expressing both wild-type CLN3 and shortened, disease-associated CLN3Δex78. “SSO-20” or “SSO-28” as discussed herein in the context of human cells or human modified oligonucleotide treatment, refers to modified oligonucleotides SSO-20 and SSO-28 of Table 10, respectively.

TABLE 10
Activity of human CLN3 with uniformly 2′-MOE
modified oligonucleotides with phosphorothioate
internucleoside linkages in human fibroblasts
(CLN3+/Δ78)

| | | | Start | Stop | | | |
|---|---|---|---|---|---|---|---|
| Com- | | | Site | Site | | | Exon |
| pound | | | on | on | | | 5 |
| Num- | | | SEQ | SEQ | | | skip- |
| ber | Com- | | ID | ID | CLN3 | SEQ | ping |
| SSO | pound | Sequence | NO: | NO: | Target | ID | total |
| ID | ID | (5′ to 3′) | 1 | 1 | Region | NO: | RNA) |
| 1 | 730441 | ACAACCCTCC | 5499 | 5516 | in- | 57 | 26 |
| | | CAACCACG | | | tron | | |
| | | | | | 4 | | |
| 2 | 730442 | GGGACAACCC | 5502 | 5519 | in- | 58 | 23 |
| | | TCCCAACC | | | tron | | |
| | | | | | 4 | | |
| 3 | 752113 | AGGGGACAAC | 5504 | 5521 | in- | 59 | 25 |
| | | CCTCCCAA | | | tron | | |
| | | | | | 4 | | |
| 4 | 752114 | CTTCCAGGGG | 5509 | 5526 | in- | 60 | 22 |
| | | ACAACCCT | | | tron | | |
| | | | | | 4 | | |
| 5 | 752115 | CAGAGCTTCC | 5514 | 5531 | in- | 61 | 71 |
| | | AGGGGACA | | | tron | | |
| | | | | | 4 | | |
| 6 | 730443 | GACCGCAGAG | 5519 | 5536 | in- | 62 | 82 |
| | | CTTCCAGG | | | tron | | |
| | | | | | 4 | | |
| 7 | 730444 | AGTGAGACCG | 5524 | 5541 | in- | 63 | 66 |
| | | CAGAGCTT | | | tron | | |
| | | | | | 4 | | |
| 8 | 730445 | AATAGAGTGA | 5529 | 5546 | in- | 64 | 89 |
| | | GACCGCAG | | | tron | | |
| | | | | | 4 | | |
| 9 | 730446 | AGGAGAATAG | 5534 | 5551 | in- | 65 | 95 |
| | | AGTGAGAC | | | tron | | |
| | | | | | 4 | | |
| 10 | 730447 | GGGACAGGAG | 5539 | 5556 | in- | 66 | 91 |
| | | AATAGAGT | | | tron | | |
| | | | | | 4 | | |
| 11 | 730448 | AGCCTGGGAC | 5544 | 5561 | in- | 67 | 90 |
| | | AGGAGAAT | | | tron | | |
| | | | | | 4/ | | |
| | | | | | exon | | |
| | | | | | 5 | | |
| 12 | 730449 | AGCACAGCCT | 5549 | 5566 | in- | 68 | 84 |
| | | GGGACAGG | | | tron | | |
| | | | | | 4/ | | |
| | | | | | exon | | |
| | | | | | 5 | | |
| 13 | 730450 | CCAGGAGCAC | 5554 | 5571 | in- | 69 | 92 |
| | | AGCCTGGG | | | tron | | |
| | | | | | 4/ | | |
| | | | | | exon | | |
| | | | | | 5 | | |
| 14 | 730451 | GTCCGCCAGG | 5559 | 5576 | exon | 70 | 95 |
| | | AGCACAGC | | | 5 | | |
| 15 | 730452 | AGGATGTCCG | 5564 | 5581 | exon | 71 | 96 |
| | | CCAGGAGC | | | 5 | | |
| 16 | 730453 | GGGAGGATGT | 5567 | 5584 | exon | 72 | 93 |
| | | CCGCCAGG | | | 5 | | |
| 17 | 752116 | TGGGGAGGAT | 5569 | 5586 | exon | 73 | 85 |
| | | GTCCGCCA | | | 5 | | |
| 18 | 752117 | GAGTGTGGGG | 5574 | 5591 | exon | 74 | 97 |
| | | AGGATGTC | | | 5 | | |
| 19 | 752118 | ATGACGAGTG | 5579 | 5596 | exon | 75 | 99 |
| | | TGGGGAGG | | | 5 | | |
| 20 | 730454 | ATTTGATGAC | 5584 | 5601 | exon | 76 | 99 |
| | | GAGTGTGG | | | 5 | | |
| 21 | 730455 | CAACAATTTG | 5589 | 5606 | exon | 77 | 96 |
| | | ATGACGAG | | | 5 | | |
| 22 | 730456 | GGAGCCAACA | 5594 | 5611 | exon | 78 | 97 |
| | | ATTTGATG | | | 5 | | |
| 23 | 730457 | CAAGAGGAGC | 5599 | 5616 | exon | 79 | 90 |
| | | CAACAATT | | | 5 | | |
| 24 | 730458 | AAGGCCAAGA | 5604 | 5621 | exon | 80 | 95 |
| | | GGAGCCAA | | | 5 | | |
| 25 | 730459 | AGGTGAAGGC | 5609 | 5626 | exon | 81 | 98 |
| | | CAAGAGGA | | | 5 | | |
| 26 | 730460 | GCAGCAGGTG | 5614 | 5631 | exon | 82 | 97 |
| | | AAGGCCAA | | | 5 | | |
| 27 | 730461 | GTAGGGCAGC | 5619 | 5636 | exon | 83 | 94 |
| | | AGGTGAAG | | | 5 | | |
| 28 | 730462 | GACCTGTAGG | 5624 | 5641 | exon | 84 | 93 |
| | | GCAGCAGG | | | 5/ | | |
| | | | | | in- | | |
| | | | | | tron | | |
| | | | | | 5 | | |
| 29 | 730463 | ACCCAGACCT | 5629 | 5646 | exon | 85 | 91 |
| | | GTAGGGCA | | | 5/ | | |
| | | | | | in- | | |
| | | | | | tron | | |
| | | | | | 5 | | |
| 30 | 730464 | CCCTCACCCA | 5634 | 5651 | exon | 86 | 61 |
| | | GACCTGTA | | | 5/ | | |
| | | | | | in- | | |
| | | | | | tron | | |
| | | | | | 5 | | |
| 31 | 730465 | CACTACCCTC | 5639 | 5656 | in- | 87 | 41 |
| | | ACCCAGAC | | | tron | | |
| | | | | | 5 | | |
| 32 | 730466 | CCTCCCACTA | 5644 | 5661 | in- | 88 | 33 |
| | | CCCTCACC | | | tron | | |
| | | | | | 5 | | |
| 33 | 730467 | CCCTGCCTCC | 5649 | 5666 | in- | 89 | 38 |
| | | CACTACCC | | | tron | | |
| | | | | | 5 | | |
| 34 | 730468 | GCCCACCCTG | 5654 | 5671 | in- | 90 | 38 |
| | | CCTCCCAC | | | tron | | |
| | | | | | 5 | | |
| 35 | 730469 | CTCCTGCCCA | 5659 | 5676 | in- | 91 | 39 |
| | | CCCTGCCT | | | tron | | |
| | | | | | 5 | | |
| 36 | 730470 | CTCAGCTCCT | 5664 | 5681 | in- | 92 | 29 |
| | | GCCCACCC | | | tron | | |
| | | | | | 5 | | |
| 37 | 730471 | CCTTTCTCAG | 5669 | 5686 | in- | 93 | 29 |
| | | CTCCTGCC | | | tron | | |
| | | | | | 5 | | |
| 38 | 730472 | CCTCCCCTTT | 5674 | 5691 | in- | 94 | 31 |
| | | CTCAGCTC | | | tron | | |
| | | | | | 5 | | |
| 39 | 730473 | CCCAGCCTCC | 5679 | 5696 | in- | 95 | 31 |
| | | CCTTTCTC | | | tron | | |
| | | | | | 5 | | |
| 40 | 730474 | GCCATCCCAG | 5684 | 5701 | in- | 96 | 31 |
| | | CCTCCCCT | | | tron | | |
| | | | | | 5 | | |

### Example 10: Dose-Dependent Effect of Uniformly 2′-MOE Modified Oligonucleotides with Phosphorothioate Internucleoside Linkages on CLN3 In Vitro

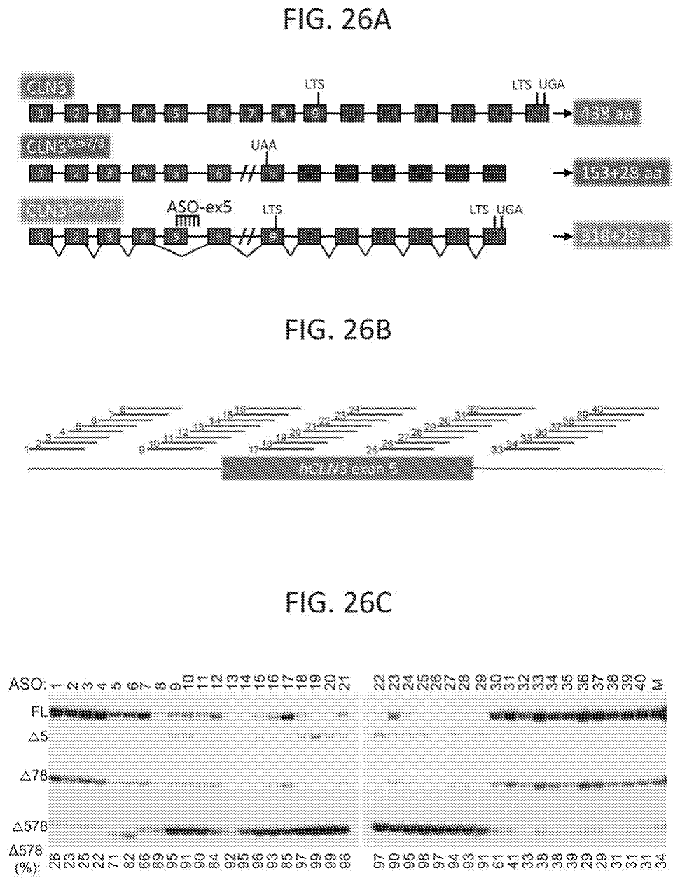

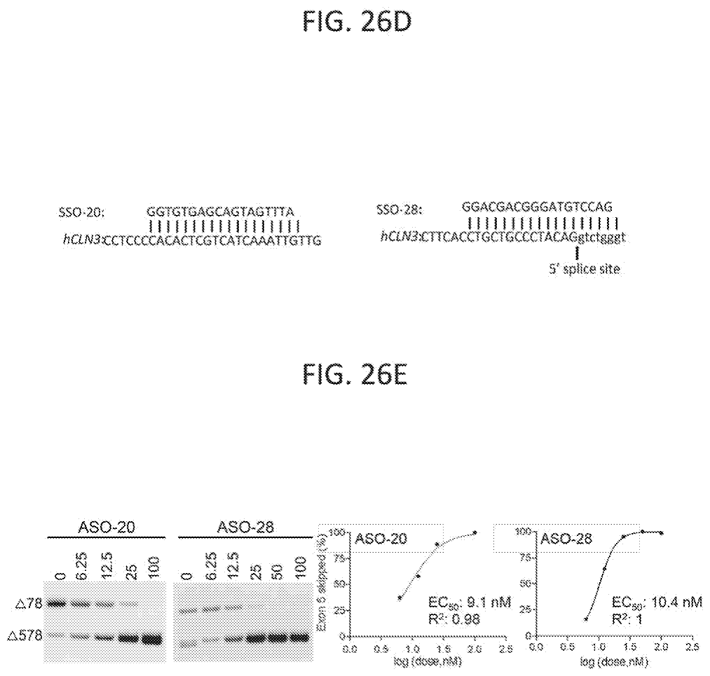

Modified oligonucleotides SSO-20 and SSO-28 were assessed in a dose response assay in a homozygous CLN3d78 patient cell line (CLN3Δ78/Δ78) (FIG. 26E). The RT-PCR analysis was performed essentially as stated herein, using the following primers: hCLN3ex4F (5′GCAACTCTGTCTCTACGGC-3′) (SEQ ID NO: 52) and hCLN3ex10R (5′CTTGAACACTGTCCACC-3′) (SEQ ID NO: 53). Table 11 below provides the percent of exon 5 skipped in relationship to the log of the dose.

TABLE 11
Activity of human CLN3 with uniformly 2′-MOE modified
oligonucleotides with phosphorothioate internucleoside
linkages in a human homozygous CLN3d78 patient cell line

| | Exon 5 skipped (%) | Exon 5 skipped (%) |
|---|---|---|
| SSO (nM) | SSO-20 | SSO-28 |
| 0 | 34.887572 | 46.06772 |
| 6.25 | 59.149406 | 54.43504 |
| 12.5 | 72.537042 | 80.1271 |
| 25 | 92.571112 | 96.39188 |
| 100 | 99.794372 | 98.88147 |

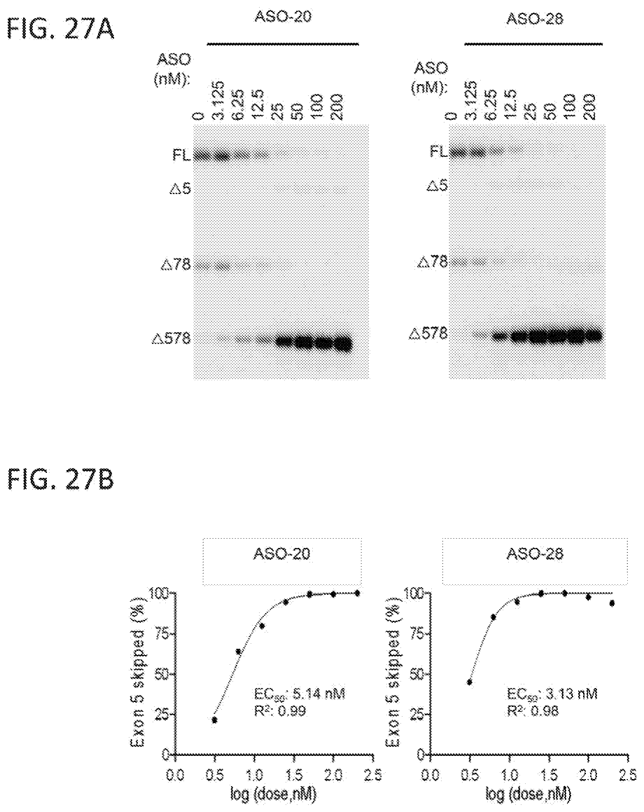

Modified oligonucleotides SSO-20 and SSO-28 were assessed in a dose response assay in a heterozygous CLN3+/Δ78 human fibroblast cell line, treated with 3.125 to 200 nM of the modified oligonucleotides. The results are provided in FIG. 27.

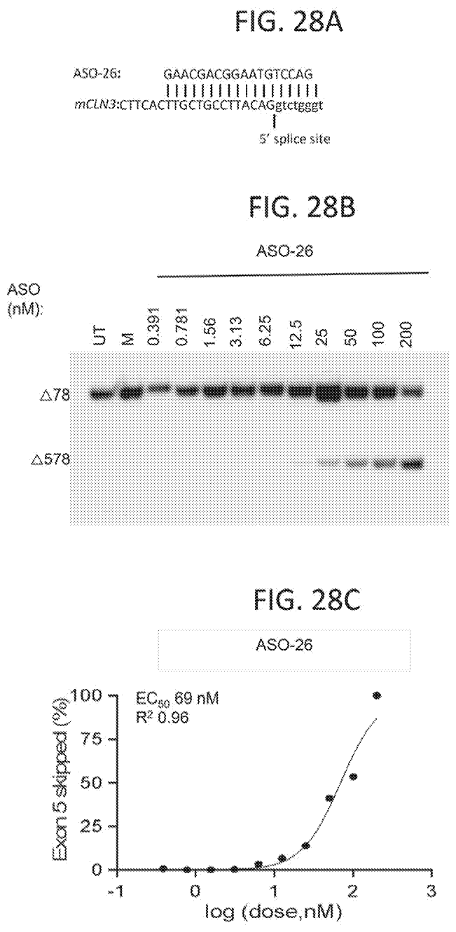

Modified oligonucleotide SSO-26 was assessed in a dose response assay in a mouse CLN3Δex7/8 mouse cell line, treated with 0.391 to 200 nM of the modified oligonucleotide. The results are provided in FIG. 28.

### Example 11: Effect of Uniformly 2′-MOE Modified Oligonucleotides With Phosphorothioate Internucleoside Linkages On Mouse CLN3 In Vivo

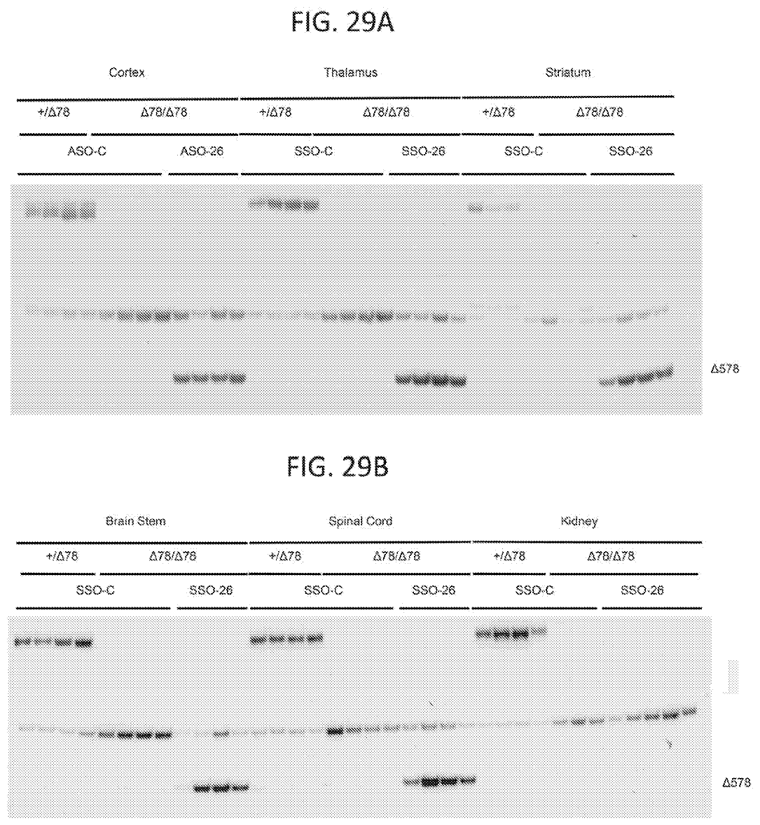

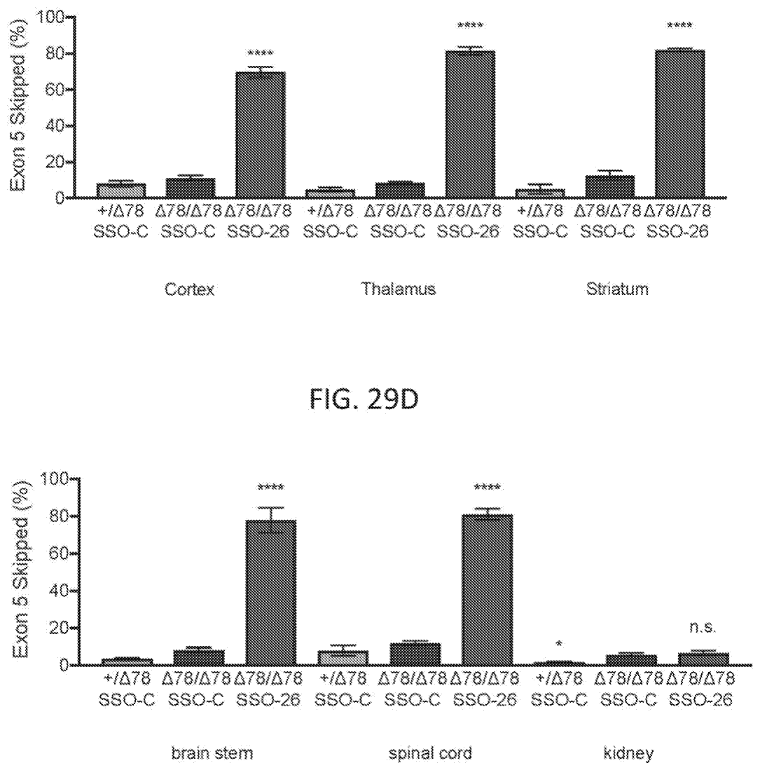

Modified oligonucleotide SSO-26, and control modified oligonucleotide SSO-C were assessed in vivo in treated mice. Following treatment, RNA was extracted from the cortex, thalamus, striatum, brain stem, spinal cord, and kidney of 19 week old heterozygous CLN3+/Δ78 and homozygous CLN3 Δ78/Δ78 mice. Quantification of exon 5 skipping showed widespread modified oligonucleotide activity in the CNS of the treated mice (FIG. 29).

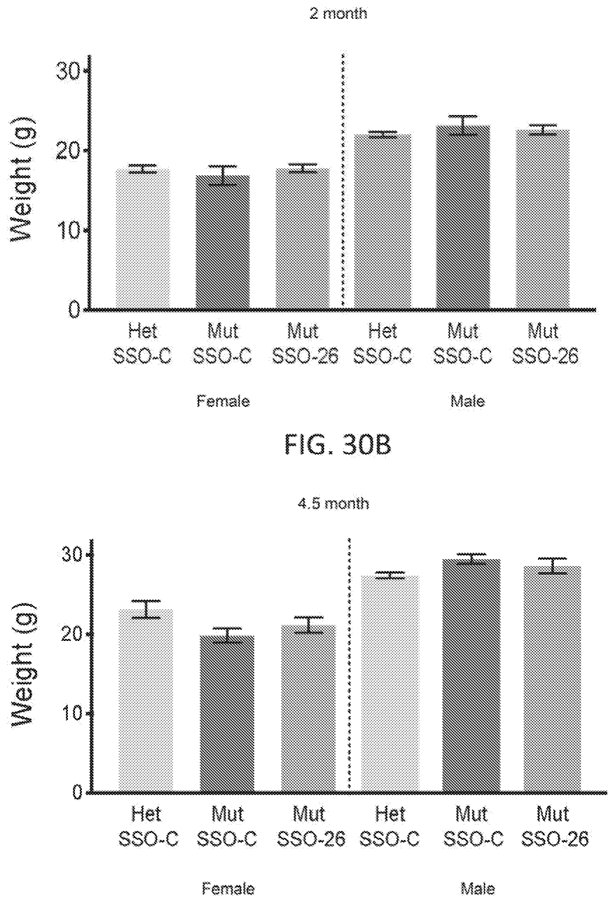

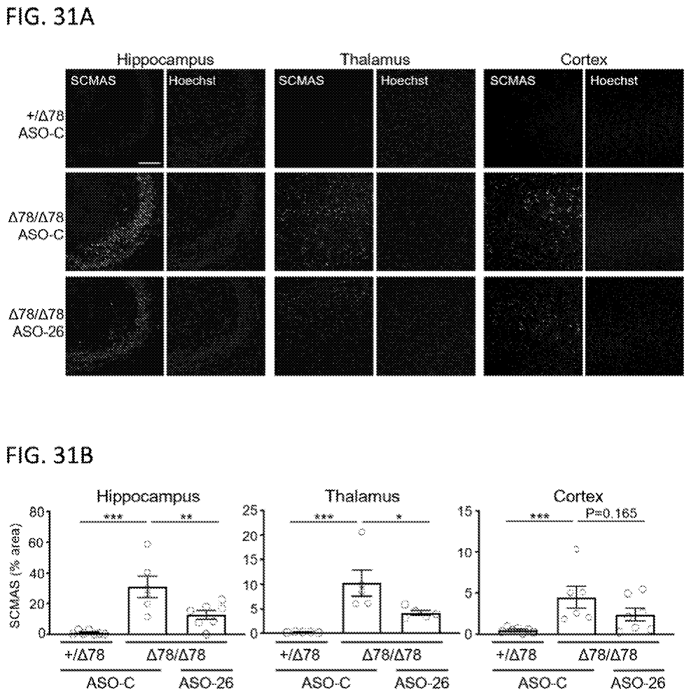

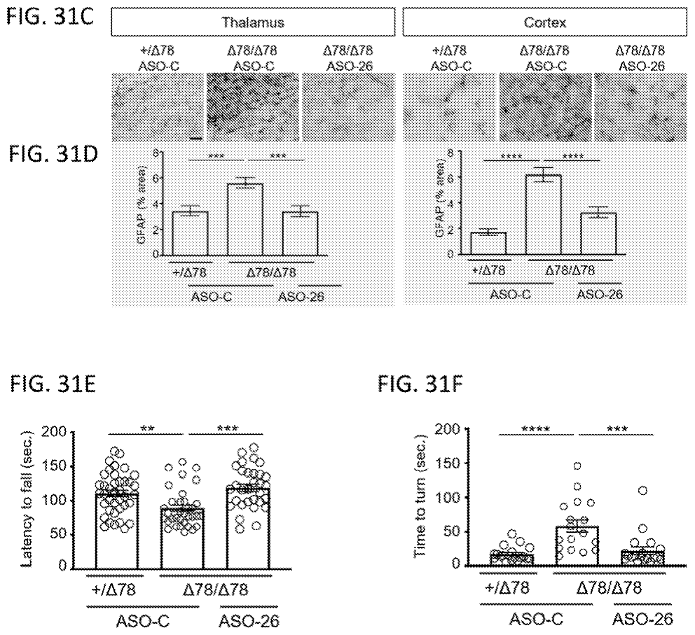

Treatment with modified oligonucleotide SSO-26 did not result in significant changes in mouse body weight, compared to treatment at days 1 or 2 post-birth with modified oligonucleotide SSO-C, when mice were assessed at 2 months and 4.5 months of age (FIG. 30).

1-137 (canceled)

138. An oligomeric compound comprising a modified oligonucleotide consisting of 12 to 50 linked nucleosides, wherein the nucleobase sequence of the modified oligonucleotide is complementary to the nucleobase sequence of an equal length portion of a target region of a human CLN3 nucleic acid, wherein the target region of the human CLN3 nucleic acid is exon 5, intron 4, or intron 5, wherein the modified oligonucleotide comprises at least one modification selected from a modified sugar moiety and a modified internucleoside linkage.

139. The oligomeric compound of claim 138, wherein the nucleobase sequence of the modified oligonucleotide is at least 95% or is 100% complementary to the nucleobase sequence of SEQ ID NO: 1 when measured across the entire nucleobase sequence of the modified oligonucleotide.

140. An oligomeric compound comprising a modified oligonucleotide consisting of 12 to 50 linked nucleosides and having a nucleobase sequence comprising a portion of at least 12 contiguous nucleobases, wherein the portion is complementary to:
an equal length portion of nucleobases 5499-5701 of SEQ ID NO: 1;
an equal length portion of nucleobases 5514-5651 of SEQ ID NO: 1;
an equal length portion of nucleobases 5519-5546 of SEQ ID NO: 1;
an equal length portion of nucleobases 5534-5646 of SEQ ID NO: 1;
an equal length portion of nucleobases 5559-5631 of SEQ ID NO: 1; or an equal length portion of nucleobases 5534-5551 of SEQ ID NO: 1;
wherein the modified oligonucleotide comprises at least one modification selected from a modified sugar moiety and a modified internucleoside linkage.

141. An oligomeric compound comprising a modified oligonucleotide consisting of 12 to 50 linked nucleosides and having a nucleobase sequence comprising at least 12, at least 13, at least 14, at least 15, at least 16, at least 17, or 18 contiguous nucleobases of the nucleobase sequence of any of SEQ ID NOS: 57-96, wherein the modified oligonucleotide comprises at least one modification selected from a modified sugar moiety and a modified internucleoside linkage.

142. The oligomeric compound of claim 138, wherein the modified oligonucleotide consists of 12 to 20, 12 to 25, 12 to 30, 13 to 20, 13 to 25, 13 to 30, 14 to 20, 14 to 25, 14 to 30, 15 to 20, 15 to 25, 15 to 30, 16 to 20, 16 to 25, 16 to 30, 17 to 20, 17 to25, 17 to 30, 18 to 20, 18 to 25, or 18 to 30 linked nucleosides.

143. The oligomeric compound of claim 138, consisting of a single-stranded modified oligonucleotide.

144. The oligomeric compound of claim 138, wherein the modified oligonucleotide comprises at least 1, at least 2, at least 3, at least 4, at least 5, at least 6, at least 7, at least 8, at least 9, at least 10, at least 11, at least 12, at least 13, at least 14, at least 15, at least 16, at least 17, or at least 18 modified nucleosides comprising a modified sugar moiety.

145. The oligomeric compound of claim 138, wherein the modified oligonucleotide comprises at least one modified nucleoside comprising a bicyclic sugar moiety having a 2′-4′ bridge, wherein the 2′-4′ bridge is selected from —O—CH₂—; and —O—CH(CH₃)—.

146. The oligomeric compound of claim 138, wherein the modified oligonucleotide comprises at least one modified nucleoside comprising a non-bicyclic modified sugar moiety comprising a 2′-MOE modified sugar or 2′-OMe modified sugar.

147. The oligomeric compound of claim 146, wherein each modified nucleoside of the modified oligonucleotide comprises a modified non-bicyclic sugar moiety comprising a 2′-MOE modified sugar or a 2′-OMe modified sugar.

148. The oligomeric compound of claim 138, wherein the modified oligonucleotide comprises at least one modified nucleoside comprising a sugar surrogate.

149. The oligomeric compound of claim 148, wherein the sugar surrogate is selected from morpholino and modified morpholino.

150. The oligomeric compound of claim 138, wherein the modified oligonucleotide comprises at least 5, at least 10, at least 15, at least 16, at least 17, or 18 modified nucleosides, each independently comprising a modified sugar moiety.

151. The oligomeric compound of claim 138, wherein the modified oligonucleotide comprises at least one modified internucleoside linkage.

152. The oligomeric compound of claim 151, wherein each internucleoside linkage of the modified oligonucleotide is a modified internucleoside linkage.

153. The oligomeric compound of claim 151, wherein at least one modified internucleoside linkage is a phosphorothioate internucleoside linkage.

154. The oligomeric compound of claim 153, wherein the modified oligonucleotide comprises at least one phosphodiester internucleoside linkage.

155. The oligomeric compound of claim 151, wherein each internucleoside linkage of the modified oligo nucleotide is either a phosphodiester internucleoside linkage or a phosphorothioate internucleoside linkage.

156. The oligomeric compound of claim 152, wherein each modified internucleoside linkage is a phosphorothioate internucleoside linkage.

157. The oligomeric compound of claim 138, wherein the modified oligonucleotide comprises at least one modified nucleobase.

158. The oligomeric compound of claim 157, wherein the modified nucleobase is a 5-methyl cytosine.

159. A pharmaceutical composition comprising an oligomeric compound of claim 138 and a pharmaceutically acceptable diluent.

160. The pharmaceutical composition of claim 159, wherein the pharmaceutically acceptable diluent is phosphate-buffered saline (PBS) or artificial cerebrospinal fluid.

161. A population of oligomeric compounds of claim 138, wherein the modified oligonucleotide comprises at least one phosphorothioate internucleoside linkage and wherein all of the phosphorothioate internucleoside linkages of the modified oligonucleotide are stereorandom.

162. A method comprising administering a pharmaceutical composition of claim 159 to an individual.

163. The method of claim 162, wherein the individual has or is at risk for developing a disease associated with CLN3.

164. The method of claim 163, wherein the disease associated with CLN3 is Batten Disease.

165. The method of claim 162, wherein at least one symptom or hallmark of the disease associated with CLN3 is ameliorated.

166. The method of claim 165, wherein the symptom or hallmark is poor motor function, seizures, vision loss, poor cognitive function, psychiatric problems, accumulation of autofluorescent ceroid lipopigment in brain tissue, brain tissue dysfunction, brain tissue cell death, accumulation of mitochondrial ATP synthase subunit C in brain tissue, accumulation of lipofuscin in brain tissue, or astrocyte activation in brain tissue.

167. The method of claim 162, wherein the individual is human.

168. A method of inducing CLN3 exon 5 skipping in a cell, comprising contacting the cell with an oligomeric compound of claim 138, and thereby inducing CLN3 exon skipping in the cell.

169. The method of claim 168, wherein the cell is a human cell.
